(19) United States
(12) Patent Application Publication
(10) Pub. No.: US 2026/0152792 A1
(43) Pub. Date: Jun. 4, 2026

(54) DNA SEQUENCING SYSTEMS AND USE THEREOF

(71) Applicant: Element Biosciences, Inc., San Diego, CA (US)

(72) Inventors: Daniel HASTINGS, San Diego, CA (US); Ben TSE, San Diego, CA (US); Russell HUDYMA, San Diego, CA (US); Alan GOMEZ, San Diego, CA (US); Jonathan LIPSHER, San Diego, CA (US); Thomas ZIEGLER, San Diego, CA (US); Jeffrey YEH, San Diego, CA (US); Michael DANGELO, San Diego, CA (US); Siyuan XING, San Diego, CA (US); Derek FULLER, San Diego, CA (US); Michael PREVITE, San Diego, CA (US); Jordan NEYSMITH, San Diego, CA (US); Cassandra NIMAN, San Diego, CA (US)

(21) Appl. No.: 19/457,949

(22) Filed: Jan. 23, 2026

Related U.S. Application Data
(63) Continuation of application No. PCT/US2024/039563, filed on Jul. 25, 2024.
(60) Provisional application No. 63/515,816, filed on Jul. 26, 2023.
(60) Provisional application No. 63/666,463, filed on Jul. 1, 2024.

(51) Int. Cl. C12Q 1/6874; B01L 3/00; G01N 35/00

(52) U.S. Cl. CPC C12Q 1/6874; B01L 3/502761; G01N 35/0099; B01L 2200/0647; B01L 2200/16; B01L 2300/0816; G01N 2035/00346

(57) ABSTRACT

The present disclosure provides flow cell devices, systems, and methods for facilitating and performing DNA sequencing analysis with reduced system complexity and cost, significant cost of goods saving, and reduced contamination level. The sequencing systems described herein permit processing of multiple flow cells simultaneously, such that sequencing and imaging steps, or multiple sequencing methods, can be performed in parallel using a single sequencing system.

### CROSS REFERENCE TO RELATED APPLICATIONS

This application is a continuation of International Application No. PCT/US2024/039563, filed on Jul. 25, 2024, which claims priority to, and the benefit of U.S. Provisional Application Nos.: 63/515,816, filed on Jul. 26, 2023 and 63/666,463, filed on Jul. 1, 2024, each of which is incorporated by reference herein in its entirety.

### BACKGROUND

In next-generation sequencing (NGS) systems, flow cell devices are used to immobilize template nucleic acid molecules derived from biological samples and then introduce a repetitive flow of sequencing reagents to attach labeled nucleotides to specific positions in the template sequences. A series of signals from the labels are detected and decoded to reveal the nucleotide sequences of the corresponding template molecules, e.g., the immobilized and/or amplified nucleic acid template molecules attached to a surface of the flow cell.

Typical NGS systems allow fluidic and thermal communications from the system to the flow cell device and sample(s) immobilized thereon during sequencing while the sample(s) remains in a fixed position relative to the optics of the sequencing system. However, leaving a flow cell in a fixed position relative to the optics of the sequencing system during steps that do not involve imaging leads to inefficient usage of optical systems, and reduces the efficiency of NGS systems. There thus exists a need in the art for compositions, systems and methods that allow asynchronous processing of multiple samples, or parts of samples, in parallel. Using the compositions, systems and methods of the disclosure, imaging and non-imaging steps on different samples can occur simultaneously, or different samples can be simultaneously subjected to different methods, thereby reducing idling times, increasing flexibility, and increasing efficiency.

### SUMMARY

Described herein are sequencing systems for sequencing nucleic acids with flexibility and scalability. The sequencing systems and methods described herein may advantageously achieve a more efficient usage of the optical system with minimum idling time, e.g., waiting for fluidic administration. The systems and methods herein may advantageously allow imaging of a sample while sequencing reactions in another sample is occurring in parallel thereby improving the throughput of the existing sequencing systems. The systems and methods described herein may advantageously separate the samples being imaged from the fluidic and/or thermal communication thereby simplifying the system architecture and enable more compact size than existing system. The systems and methods described herein may also advantageously enable independent fluidic and/or thermal communications to various samples thereby allowing users to image samples that use different reagent(s) or sequencing protocols and possibly combining them within a single sequence run.

The disclosure provides sequencing system comprising: an optical system 2020 comprising an objective lens; a x-y stage 2010 configured to hold a sample to be imaged thereon and to move the sample within an x-y plane relative to the objective lens, wherein the sample is immobilized on one or more flow cell devices; a nest bank 2050 configured to provide fluidic and thermal communication to the sample when the one or more flow cell devices are coupled to the nest bank; and a moving mechanism 2040, optionally comprising a movable arm configured to move the one or more flow cell devices between the x-y stage 2010 and the nest bank 2050 during a sequence run.

In some embodiments, the x-y stage 2010 is actuated automatically by a first actuator with a first spatial precision.

In some embodiments, the movable arm is actuated automatically by a second actuator with a second spatial precision. In some embodiments, the first actuator, the second actuator, or both is controlled by one or more hardware processors of the sequencing system.

In some embodiments, the sequencing system further comprises: a housing configured for holding one or more of the optical system 2020, the x-y stage 2010, the nest bank 2050, and the moving mechanism 2040 therewithin.

In some embodiments, the movable arm is actuated automatically to move in three dimensions (3D). In some embodiments, movement in each of the three dimensions are of one or more predetermined spatial precision.

In some embodiments, the sequencing system lacks fluidic communication or thermal communication at or near the x-y stage 2010 to the one or more flow cell devices when the flow cell devices are immobilized on the x-y stage 2010.

In some embodiments, each of the one or more flow cell devices comprises an open landing area configured for receiving fluids openly from the nest bank 2050. In some embodiments, the flow cell device comprises a plurality of microfluidic channels, and the nest bank 2050 is configured to allow fluidic communication to each of the plurality of microfluidic channel independently and simultaneously. In some embodiments, the flow cell device comprises a plurality of microfluidic channels, and the nest bank 2050 is configured to allow fluidic communication to each of the plurality of microfluidic channel independently and sequentially. In some embodiments, the flow cell device comprises a plurality of microfluidic channels, and the nest bank 2050 is configured to allow fluidic communication to each of the plurality of microfluidic channel independently without cross-contamination.

In some embodiments, the x-y stage 2010 is actuated to move within the x-y plane for a predetermined distance. In some embodiments, the predetermined distance is based on the distance between two adjacent microfluidic channels of the flow cell device.

In some embodiments, the nest bank 2050 is configured to enable fluidic and thermal communication with the one or more flow cell devices. In some embodiments, the nest bank 2050 is configured to enable fluidic and thermal communication with at least 1, 2, 3, 4, 5, 6, 7, 8, 9 or 10 flow cell devices when each of the flow cell devices is in a locked position with the nest bank 2050. In some embodiments, the nest bank 2050 is configured to hold each of the flow cell devices in a unlocked position in which the flow cell device is removable from the nest bank 2050 and a locked position in which the flow cell device is spatially registered to the nest bank 2050, fixedly coupled to the nest bank 2050, and sealed fluidic communication and thermal communication between the nest bank and the flow cell device are enabled.

In some embodiments, the flow cell device is coupled to a carrier 2051. In some embodiments, the movable arm is configured to move the carrier 2051 and the flow cell device together. In some embodiments, the carrier 2051 is configured to be spatially registered to the nest bank in the locked position. In some embodiments, the nest bank 2050 comprises one or more fasteners. In some embodiments, the one or more fasteners use magnetic force. In some embodiments, the one or more fasteners comprises a rare earth magnet, an electromagnetic coil, or both. In some embodiments, the one or more fasteners are controlled by one or more processors to switch between a on-stage and an off-stage. In some embodiments, the one or more fasteners lack mechanical fasteners.

In some embodiments, the movable arm is configured to move the one or more flow cell devices between the x-y stage 2010 and the nest bank 2050 with a first spatial precision. In some embodiments, the movable arm comprises a grabber that is configured to grab a carrier 2051 when the carrier 2051 is in a decoupled position in relation to the nest bank 2050 or when the carrier 2051 is in the decoupled position in relation to the x-y stage 2010. In some embodiments, the movable arm comprises a horizontal arm that is mechanically supported by a vertical arm. In some embodiments, the movable arm comprises an upper arm, a joint, a forearm, a wrist, and a grabber attached to the forearm. In some embodiments, the movable arm is configured to move with 6 degrees of freedom. In some embodiments, the grabber is movably attached to the horizontal or vertical arm. In some embodiments, the grabber is configured to move in 3D. In some embodiments, the moving mechanism 2040 comprises a plurality of tracks, each track connecting a carrier 2051 coupled to the nest bank to the x-y stage 2010. In some embodiments, the grabber is configured to hold the flow cell device carrier via frictional, electromagnetic, or magnetic force.

In some embodiments, the carrier 2051 comprises one or more sensors. In some embodiments, the x-y stage 2010 comprises one or more sensors. In some embodiments, the nest bank 2050 comprises one or more sensors. In some embodiments, the one or more sensors are configured to provide feedback to a processor that facilitates positioning of the carrier 2051 relative to the x-y stage 2010, the optical system 2020, or the nest bank 2050.

In some embodiments, the moving mechanism 2040 comprises one or more belt conveyors.

In some embodiments, the plurality of tracks comprises one or more actuators configured to actuate one or more of the plurality of tracks to move corresponding carriers 2051 to the x-y stage 2010.

In some embodiments, the x-y stage 2010 is configured to be actuated to move to a 3D position with a second spatial precision. In some embodiments, the second spatial precision is greater than the first spatial precision by 2×, 3×, 4×, 5×, 6×, 7×, 8×, 9×, or 10×.

In some embodiments, the x-y stage 2010 comprises: a fastener configured to removably secure the flow cell device thereto. In some embodiments, the fastener comprises one or more clamps.

In some embodiments, each carrier 2051 comprises a coupled position in which the carrier 2051 is removably attached to the x-y stage 2010. In some embodiments, each carrier 2051 comprises a decoupled position in which the carrier 2051 is removable from the x-y stage 2010.

In some embodiments, the x-y stage 2010 comprises one or more pumps configured to extract fluids from the flow cell device when the corresponding carrier 2051 is coupled to the x-y stage 2010. In some embodiments, the x-y stage 2010 comprises a heating device, a cooling device, or both. In some embodiments, the x-y stage 2010 is coupled to a mechanical decoupler that is configured to isolate the x-y stage from vibration or mechanical disturbance external to the x-y stage 2010.

In some embodiments, the nest bank 2050 comprises one or more fasteners, each configured to fasten a corresponding carrier 2051 to the nest bank 2050. In some embodiments, each fastener comprises one or more clamps. In some embodiments, the one or more clamps are actuated by magnetic or electromagnetic force or pressure.

In some embodiments, the nest bank 2050 comprises one or more pumps configured to extract fluids from the flow cell device when the corresponding carrier 2051 is coupled to the nest bank 2050.

In some embodiments, each carrier 2051 comprises a decoupled position in which the carrier 2051 is removable from the nest bank 2050. In some embodiments, each carrier 2051 comprises a coupled position in which the flow cell device carrier is removably attached to the nest bank 2050, and in sealed fluidic communication with the nest bank 2050.

In some embodiments, the nest bank 2050 comprises a 3D movement device that is configured to position the carrier 2051 relative to the nest bank with a third spatial precision. In some embodiments, the third spatial position is greater than the first spatial precision by 2×, 3×, 4×, 5×, 6×, 7×, 8×, 9×, or 10×.

In some embodiments, the carrier 2051 comprises: an opening at a surface of the carrier 2051 configured to receive a flow cell device therein. In some embodiments, the carrier 2051 comprises: one or more fluidic pathways in sealed fluidic communication with the flow cell device when the flow cell device is removably attached to the carrier 2051. In some embodiments, the carrier 2051 comprises: a pump configured to pull or push fluids between the flow cell device and the carrier 2051. In some embodiments, the carrier 2051 comprises: a valve positioned between a fluidic pathway connecting to the flow cell device and a port opening of the carrier 2051, wherein the valve that is in an open position when the flow cell device is in the coupled position to the carrier 2051; and in a closed position when the flow cell device is in the decoupled position. In some embodiments, the carrier 2051 comprises: a port opening with a connector that is configured to enable sealed fluidic communication between the carrier 2051 and the corresponding nest module when the connector is in a connected position. In some embodiments, the carrier 2051 comprises: electric wiring with an electric connector configured to enable electric communication between the carrier 2051 and a power supply. In some embodiments, the carrier 2051 comprises: a battery, a sensor, or both, and wherein the battery or sensor is connected with the electric connector via the electric wiring.

In some embodiments, the nest bank 2050 comprises one or more reagent containers. In some embodiments, the one or more reagent containers are disposable.

In some embodiments, the moving mechanism 2040 is configured to dip a flow cell device into at least some of the one or more reagent containers.

In some embodiments, the nest bank 2050 further comprises: a cooler, a heater, or both. In some embodiments, the cooler or heater is configured to control temperature of each sample immobilized on the one or more flow cell devices. In some embodiments, the cooler or heater comprises one or more of: a fan configured to blow cool or hot air; a microwave, an infrared light source, and an electromagnetic wave source.

In some embodiments, the sequencing system further comprises a beam dump configured to absorb at least some excitation light generated by the optical system. In some embodiments, the sequencing system further comprises a beam dump configured to prevent at least some excitation light from reaching an imaging sensor of the optical system. In some embodiments, the beam dump is displaced from the flow cell device by a gap zone. In some embodiments, the beam dump contacts the flow cell device with a predetermined latching force. In some embodiments, the beam dump contacts the x-y stage with a predetermined damping force. In some embodiments, the predetermined damping force is configured to reduce the predetermined latching force so that a net force on the flow cell device can be customized to be within a predetermined range.

The disclosure provides a sequencing method comprising: (a) moving a first flow cell device from a nest bank 2050 to a x-y stage 2010, wherein the first flow cell device comprises a first sample immobilized thereon; (b) moving the x-y stage 2010 and the first sample thereon relative to an objective lens of an optical system of a sequencing system; (c) imaging the first sample immobilized on the first flow cell device on the x-y stage using the optical system 2020; (d) moving the first flow device from the x-y stage 2010 to the nest bank 2050; (e) simultaneously allowing fluidic and thermal communication between the nest bank 2050 and a second flow cell device during one or more of: (a)-(d); and (f) moving a second flow cell device from the nest bank 2050 to the x-y stage 2010, wherein the second flow cell device comprises a second sample immobilized thereon; (g) moving the x-y stage 2010 and the second sample thereon relative to an objective lens of the optical system 2040 of the sequencing system; (h) imaging the second sample immobilized on the second flow cell device on the x-y stage 2010 using the optical system 2040; (i) moving the first flow device from the x-y stage 2010 to the nest bank 2050; and (j) simultaneously allowing fluidic and thermal communication between the nest bank 2050 and the first flow cell device during one or more of: (f)-(i).

The disclosure provides a sequencing method comprising (a) moving a first flow cell device from a nest bank 2050 to a x-y stage 2010, wherein the first flow cell device comprises a first sample immobilized thereon; (b) moving the x-y stage 2010 and the first sample thereon relative to an objective lens of an optical system 2020 of a sequencing system; (c) imaging the first sample immobilized on the first flow cell device on the x-y stage 2010 using the optical system 2020; (d) moving the first flow device from the x-y stage 2010 to the nest bank 2050; (e) simultaneously allowing fluidic and thermal communication between the nest bank 2050 and a second flow cell device during one or more of: (a)-(d); and (f) moving the second flow cell device from the nest bank 2050 to the x-y stage 2010, wherein the second flow cell device comprises a second sample immobilized thereon.

In some embodiments, the sequencing method further comprises: repeating operations (a)-(e). In some embodiments, the sequencing method further comprises: repeating operations (f)-(j). In some embodiments, the sequencing method further comprises: repeating operations (a)-(j) for a number of repetitions. In some embodiments, the number of repetitions is in a range from 1 to 500.

In some embodiments, allowing fluidic communication between the nest bank 2050 and the first flow cell device comprises: reversibly fastening the flow cell device to a carrier 2051 via one or more fasteners to enable sealed fluidic communication between the flow cell device and the carrier 2051; and reversibly fastening the carrier 2051 to the nest bank 2050 via the one or more fasteners to enable sealed fluidic communication between the nest bank 2050 and the carrier 2051 and to enable physical contact to heat dissipation elements.

In some embodiments, (a) moving the first flow cell device from the nest bank 2050 to the x-y stage 2010 is within a first flow cycle of a sequence run and (f) moving the first flow cell device from the nest bank 2050 to the x-y stage 2010 is within a second flow cycle of the sequencing run different from the first flow cycle.

In some embodiments, each of the operations of: (a)-(b) and (d)-(g) is completed within less than 0.5 seconds, 1 second, 2 seconds, 3 seconds, 4 seconds, 5 seconds, 6 seconds, 7 seconds, 8 seconds, 9 seconds, or 10 seconds. In some embodiments, each of the operations of: (a)-(b) and (d)-(g) is completed within less than 0.5 seconds, 1 second, 2 seconds, or 3 seconds.

In some embodiments, (e) simultaneously allowing fluidic and thermal communication between the nest bank 2050 and the first flow cell device during one or more of: (a)-(d) comprises: turning the one or more fasteners into an on-stage to enable sealed fluidic communication and physical contact for thermal communication. In some embodiments, (e) simultaneously allowing fluidic and thermal communication between the nest bank and the first flow cell device during one or more of: (a)-(d) comprises: dipping the flow cell device into at least some of the one or more reagent containers in a predetermined sequence.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a block diagram of a computer-implemented system for performing operations in DNA sequencing and sequencing analysis, according to some embodiments.

FIG. 2 is a schematic showing of an exemplary sequencing system 2000, comprising, in this case, an optical system 2020 comprising an objective lens, an x-y stage 2010, a nest bank 2050, flow cell device carriers 2051 and a moving mechanism 2040 (e.g., a movable arm), surrounded by a housing 2030, according to some embodiments.

FIG. 3 is a schematic that shows an exemplary nest bank module, according to some embodiments.

FIGS. 4A-4B are both schematics that show the exemplary nest bank module in FIG. 3, according to some embodiments.

FIGS. 5A-5B are both schematics that show an exemplary flow cell device, according to some embodiments.

FIG. 6 shows an exemplary nest bank with reagent containers, according to some embodiments. In FIG. 6, each reagent container is at an appropriate temperature for a given reaction. Reagent containers can be part of the disposable (dry) instrument.

FIG. 7 shows an exemplary flow cell device with an open landing area, according to some embodiments.

FIG. 8 illustrates a block diagram of a computer system for fluidic control and for performing sequencing and sequencing analysis, according to some embodiments.

FIG. 9 is a schematic showing an exemplary linear single stranded library molecule (900) which comprises: a surface pinning primer binding site (920); an optional left unique identification sequence (980); a left index sequence (960); a forward sequencing primer binding site (940); an insert region having a sequence of interest (910); reverse sequencing primer binding site (950); a right index sequence (970); and a surface capture primer binding site (930).

FIG. 10 is a schematic showing an exemplary linear single stranded library molecule (900) which comprises: a surface pinning primer binding site (920); a left index sequence (960); a forward sequencing primer binding site (940); an insert region having a sequence of interest (910); a reverse sequencing primer binding site (950); a right index sequence (970); an optional right unique identification sequence (990); and a surface capture primer binding site (930).

FIG. 11 is a schematic of various exemplary configurations of multivalent molecules. Left (Class I): schematics of multivalent molecules having a “starburst” or “helter-skelter” configuration. Center (Class II): a schematic of a multivalent molecule having a dendrimer configuration. Right (Class III): a schematic of multiple multivalent molecules formed by reacting streptavidin with 4-arm or 8-arm PEG-NHS with biotin and dNTPs. Nucleotide units are designated ‘N’, biotin is designated ‘B’, and streptavidin is designated ‘SA’.

FIG. 12 is a schematic of an exemplary multivalent molecule comprising a generic core attached to a plurality of nucleotide-arms.

FIG. 13 is a schematic of an exemplary multivalent molecule comprising a dendrimer core attached to a plurality of nucleotide-arms.

FIG. 14 shows a schematic of an exemplary multivalent molecule comprising a core attached to a plurality of nucleotide-arms, where the nucleotide arms comprise biotin, a spacer, a linker and a nucleotide unit.

FIG. 15 is a schematic of an exemplary nucleotide-arm comprising a core attachment moiety, a spacer, a linker and a nucleotide unit.

FIG. 16 shows the chemical structure of an exemplary spacer (top), and the chemical structures of various exemplary linkers, including an 11-atom Linker, 16-atom Linker, 23-atom Linker and an N3 Linker (bottom).

FIG. 17 shows the chemical structures of various exemplary linkers, including Linkers 1-9.

FIG. 18 shows the chemical structures of various exemplary linkers joined/attached to nucleotide units.

FIG. 19 shows the chemical structures of various exemplary linkers joined/attached to nucleotide units.

FIG. 20 shows the chemical structures of various exemplary linkers joined/attached to nucleotide units.

FIG. 21 shows the chemical structures of various exemplary linkers joined/attached to nucleotide units.

FIG. 22 shows the chemical structure of an exemplary biotinylated nucleotide-arm. In this example, the nucleotide unit is connected to the linker via a propargyl amine attachment at the 5 position of a pyrimidine base or the 7 position of a purine base.

FIG. 23 shows a schematic illustration of one embodiment of the flow cell devices in which the support comprises a glass substrate and alternating layers of hydrophilic coatings which are covalently or non-covalently adhered to the glass, and which further comprises chemically-reactive functional groups that serve as attachment sites for oligonucleotide primers.

FIGS. 24A-24B each show a schematic illustration of an exemplary embodiment of the nest module, a part of the nest bank 2050 that receives the carrier 2051 when the carrier 2051 is coupled with the nest bank 2050.

FIG. 25 is a schematic illustration of movement of the carrier 2051 between the optical system 2020 and the nest bank 2050.

FIG. 26A shows a schematic illustration of an x-y stage 2010 of the optical system in FIG. 25.

FIG. 26B shows schematic illustration of the nest bank 2050 in FIG. 25.

FIGS. 27A-27C each show a schematic illustration of exemplary embodiments of the moving mechanism 2040 in relation to the optical system 2020 and the nest bank 2050.

FIG. 28 shows a series of schematic illustrations of an exemplary embodiment of the sequencing system.

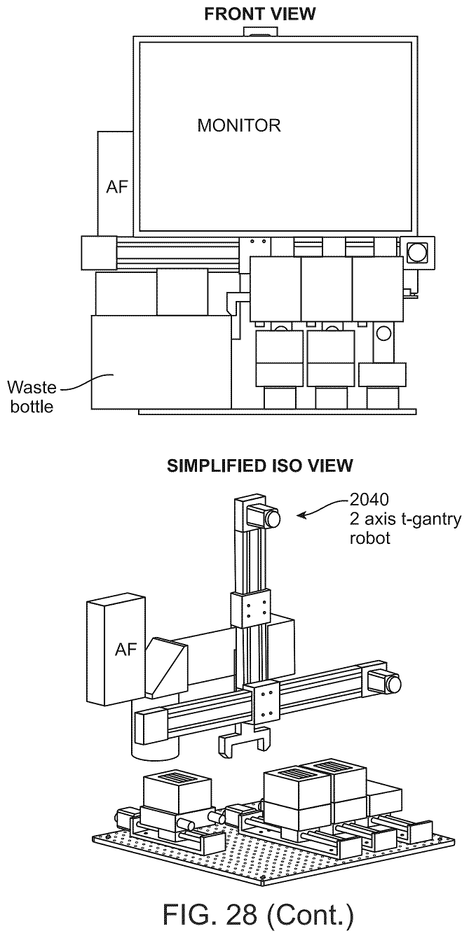

FIG. 29 shows a schematic illustration of an exemplary embodiment of the flow cell device carrier.

FIG. 30 shows a schematic illustration of an exemplary embodiment of movement of the flow cell device carrier 2051 between the optical system 2020 and the nest bank 2050.

FIG. 31 shows a schematic illustration of an exemplary embodiment of the flow cell device carrier 2051 in a coupled position with the nest bank 2050 for fluidic communication.

FIGS. 32A-32B each show exemplary embodiments of the flow cell device carrier 2051 in a coupled position with the x-y stage 2010 for imaging.

### DETAILED DESCRIPTION

Described herein are systems and devices to analyze different nucleic acid sequences, e.g. from amplified nucleic acid arrays in flow cells or from an array of immobilized nucleic acids. The systems and devices described herein can also be useful in, e.g., sequencing for comparative genomics, tracking gene expression, microRNA sequence analysis, epigenomics, and aptamer and phage display library characterization, as well as other sequencing applications. The systems and devices herein comprise various combinations of optical, mechanical, fluidic, thermal, electrical, and computing devices/aspects.

The advantages of the disclosed flow cell devices, fluidic control devices, systems and methods include, but are not limited to: flexible and scalable system throughput and flexible adaptation of the systems to different sequencing applications; reduced device and system manufacturing/maintenance complexity and cost; reduced optics idling time; and separation of fluidic and thermal communications from the sample during imaging thereby reducing possible interferences to imaging results.

The design features of some disclosed capillary flow cell devices, flow cell device carriers, and systems include, but are not limited to: an open dispensing tip in the fluidic control device and an open landing area on the flow cell device to allow open delivery of reagents without the complexity and cost of traditional tubing and to enable flexibility in the systems to adapt to different sequencing applications; a moving mechanism (e.g., a movable arm) that can move the sample(s) relative to the optics to maximize utilization of the optical system and reduce time needed to complete a sequence run; fluidic and thermal communication localized to the nest bank but absent from the x-y stage thereby reducing system size and removing possible interferences from them during imaging; a combination of coarse and efficient movement of the sample by the movable arm and fine movement and tuning of the sample relative to the objective lens or the dispensing tips to ensure accurate and efficient alignment of the sample(s) for imaging and for fluidic administration.

Although the disclosed flow cell devices, systems and methods are described primarily in the context of their use for nucleic acid sequencing applications, various aspects of the disclosed devices and systems may be applied not only to nucleic acid sequencing but also to any other type of chemical analysis, biochemical analysis, nucleic acid analysis, cell analysis, or tissue analysis application. It shall be understood that different aspects of the disclosed devices and systems can be appreciated individually, collectively, or in combination with each other.

### Sequencing Systems

Disclosed herein, in some embodiments, are flow cell devices and systems that can be employed for performing or facilitating DNA sequencing analysis using sequencing systems.

The sequencing systems may utilize various sequencing techniques including but not limited to the sequencing techniques disclosed herein.

### Definitions

The headings provided herein are not limitations of the various aspects of the disclosure, which aspects can be understood by reference to the specification as a whole. Unless defined otherwise, technical and scientific terms used herein have meanings that are commonly understood by those of ordinary skill in the art unless defined otherwise. Generally, terminologies pertaining to techniques of molecular biology, nucleic acid chemistry, protein chemistry, genetics, microbiology, transgenic cell production, and hybridization described herein are those well-known and commonly used in the art. Techniques and procedures described herein are generally performed according to conventional methods well known in the art and as described in various general and more specific references that are cited and discussed throughout the instant specification. For example, see Sambrook et al., Molecular Cloning: A Laboratory Manual (Third ed., Cold Spring Harbor Laboratory Press, Cold Spring Harbor, N.Y. 2000). See also Ausubel et al., Current Protocols in Molecular Biology, Greene Publishing Associates (1992). The nomenclatures utilized in connection with, and the laboratory procedures and techniques described herein are those well-known and commonly used in the art.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative term (e.g., “or”) is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include: “A and B”; “A or B”; “A” (A alone); and “B” (B alone). In a similar manner, the term “and/or” as used in a phrase such as “A, B, and/or C” is intended to encompass each of the following aspects: “A, B, and C”; “A, B, or C”; “A or C”; “A or B”; “B or C”; “A and B”; “B and C”; “A and C”; “A” (A alone); “B” (B alone); and “C” (C alone).

As used herein and in the appended claims, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the terms “about,” “approximately,” and “substantially” refer to a value or composition that is within an acceptable error range for the particular value or composition as determined by one of ordinary skill in the art, which will depend in part on how the value or composition is measured or determined, i.e., the limitations of the measurement system. For example, “about,” “approximately,” or “substantially” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about,” “approximately,” “substantially” should be assumed to be within an acceptable error range for that particular value or composition. Also, where ranges and/or subranges of values are provided, the ranges and/or subranges can include the endpoints of the ranges and/or subranges.

The term “glass” refers to silica-based material, including silicate, borosilicate, fused silica, fused quartz, glass, quartz, or lead glass.

The term “polony” used herein refers to a nucleic acid library molecule that can be clonally amplified in-solution or on-support to generate an amplicon that can serve as a template molecule for sequencing. For example, a linear library molecule can be circularized to generate a circularized library molecule, and the circularized library molecule can be clonally amplified in-solution or on-support to generate a concatemer molecule. The concatemer can serve as a nucleic acid template molecule which can be sequenced. The concatemer is sometimes referred to as a polony. In some embodiments, a polony includes nucleotide strands.

As used herein, the term “clonally amplified” and it variants refers to a nucleic acid template molecule that has been subjected to one or more amplification reactions either in-solution or on-support. In the case of in-solution amplified template molecules, the resulting amplicons can be distributed onto the support. Prior to amplification, the template molecule typically comprises a sequence of interest and at least one universal adaptor sequence, i.e. a sequence common to all the template molecules in reaction or from a specific sample. In some embodiments, clonal amplification comprises the use of a polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification (RCA), circle-to-circle amplification, helicase-dependent amplification, recombinase-dependent amplification, single-stranded binding (SSB) protein-dependent amplification, or any combination thereof.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules that have not yet been subjected to post-translation modification such as proteolytic cleavage, cleavage due to ribosomal skipping, hydroxylation, methylation, lipidation, acetylation, SUMOylation, ubiquitination, glycosylation, phosphorylation and/or disulfide bond formation. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins.

As used herein, the term “sequencing” and its variants comprise obtaining sequence information from a nucleic acid strand, typically by determining the identity of at least some nucleotides (including their nucleobase components) within a nucleic acid template molecule. While in some embodiments, “sequencing” a given region of a nucleic acid template molecule includes identifying each and every nucleotide within the region that is sequenced, in some embodiments “sequencing” comprises methods whereby the identity of only some of the nucleotides in the region is determined, while the identity of some nucleotides remains undetermined or incorrectly determined. Any suitable method of sequencing may be used. In an exemplary embodiment, sequencing can include label-free or ion based sequencing methods. In some embodiments, sequencing can include labeled or dye-containing nucleotide or fluorescent based nucleotide sequencing methods. In some embodiments, sequencing can include polony-based sequencing or bridge sequencing methods. In some embodiments, sequencing includes massively parallel sequencing platforms that employ sequence-by-synthesis, sequence-by-hybridization or sequence-by-binding procedures. Examples of massively parallel sequence-by-synthesis procedures include polony sequencing, pyrosequencing (e.g., from 454 Life Sciences; U.S. Pat. Nos. 7,211,390, 7,244,559 and 7,264,929), chain-terminator sequencing (e.g., from Illumina; U.S. Pat. No. 7,566,537; Bentley 2006 Current Opinion Genetics and Development 16:545-552; and Bentley, et al., 2008 Nature 456:53-59, ion-sensitive sequencing (e.g., from Ion Torrent), probe-anchor ligation sequencing (e.g., Complete Genomics), DNA nanoball sequencing, nanopore DNA sequencing. Examples of single molecule sequencing include Heliscope single molecule sequencing, and single molecule real time (SMRT) sequencing from Pacific Biosciences (Levene, et al., 2003 Science 299(5607):682-686; Eid, et al., 2009 Science 323(5910): 133-138; U.S. Pat. Nos. 7,170,050; 7,302,146; and 7,405,281). An example of sequence-by-hybridization includes SOLiD sequencing (e.g., from Life Technologies; WO 2006/084132). An example of sequence-by-binding includes Omniome sequencing (e.g., U.S Pat. No. 10,246,744).

The term “polymerase” and its variants, as used herein, comprises any enzyme that can catalyze polymerization of nucleotides (including analogs thereof) into a nucleic acid strand. Typically, but not necessarily, such nucleotide polymerization can occur in a template-dependent fashion. Typically, a polymerase comprises one or more active sites at which nucleotide binding and/or catalysis of nucleotide polymerization can occur. In some embodiments, a polymerase includes other enzymatic activities, such as for example, 3′ to 5′ exonuclease activity or 5′to 3′exonuclease activity. In some embodiments, a polymerase has strand displacing activity. A polymerase can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze nucleotide polymerization (e.g., catalytically active fragment). In some embodiments, a polymerase can be isolated from a cell, or generated using recombinant DNA technology or chemical synthesis methods. In some embodiments, a polymerase can be expressed in prokaryote, eukaryote, viral, or phage organisms. In some embodiments, a polymerase can be post-translationally modified proteins or fragments thereof. A polymerase can be derived from a prokaryote, eukaryote, virus or phage. A polymerase comprises DNA-directed DNA polymerase and RNA-directed DNA polymerase.

As used herein, the term “fidelity” refers to the accuracy of DNA polymerization by template-dependent DNA polymerase. The fidelity of a DNA polymerase is typically measured by the error rate (the frequency of incorporating an inaccurate nucleotide, i.e., a nucleotide that is not complementary to the template nucleotide). The accuracy or fidelity of DNA polymerization is maintained by both the polymerase activity and the 3′-5′ exonuclease activity of a DNA polymerase.

As used herein, the term “binding complex” refers to a complex formed by binding together a nucleic acid duplex, a polymerase, and a free nucleotide or a nucleotide unit of a multivalent molecule, where the nucleic acid duplex comprises a nucleic acid template molecule hybridized to a nucleic acid primer. In the binding complex, the free nucleotide or nucleotide unit may or may not be bound to the 3′ end of the nucleic acid primer at a position that is opposite a complementary nucleotide in the nucleic acid template molecule. A “ternary complex” is an example of a binding complex which is formed by binding together a nucleic acid duplex, a polymerase, and a free nucleotide or nucleotide unit of a multivalent molecule, where the free nucleotide or nucleotide unit is bound to the 3′ end of the nucleic acid primer (as part of the nucleic acid duplex) at a position that is opposite a complementary nucleotide in the nucleic acid template molecule.

As used herein, a “nucleotide unit” or ‘nucleotide moiety” refers to nucleotides (e.g., dATP, dTTP, dGTP, dCTP, or dUTP), or analogs thereof, comprising comprises a base, sugar and at least one phosphate group. Nucleotide units can be attached to the multivalent molecules used in the sequencing reactions described herein. In general, all nucleotide units attached to the same multivalent molecule will have the same identity (e.g., all A, all T, all C, or all G), although the skilled artisan will appreciate that there may be situations in which a multivalent molecule comprising nucleotide units of differing identity will be advantageous.

The term “persistence time” and related terms refers to the length of time that a binding complex remains stable without dissociation of any of the components, where the components of the binding complex include a nucleic acid template and nucleic acid primer, a polymerase, a nucleotide unit of a multivalent molecule or a free (e.g., unconjugated) nucleotide. The nucleotide unit or the free nucleotide can be complementary or non-complementary to a nucleotide residue in the template molecule. The nucleotide unit or the free nucleotide can bind to the 3′ end of the nucleic acid primer at a position that is opposite a complementary nucleotide residue in the nucleic acid template molecule. The persistence time is indicative of the stability of the binding complex and strength of the binding interactions. Persistence time can be measured by observing the onset and/or duration of a binding complex, such as by observing a signal from a labeled component of the binding complex. For example, a labeled nucleotide or a labeled reagent comprising one or more nucleotides may be present in a binding complex, thus allowing the signal from the label to be detected during the persistence time of the binding complex. One exemplary label is a fluorescent label. The binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and chimeric forms containing DNA and RNA. Nucleic acids can be single-stranded or double-stranded. Nucleic acids comprise polymers of nucleotides, where the nucleotides include natural or non-natural bases and/or sugars. Nucleic acids comprise naturally-occurring internucleosidic linkages, for example phosphodiester linkages. Nucleic acids comprise non-natural internucleoside linkages, including phosphorothioate, phosphorothiolate, or peptide nucleic acid (PNA) linkages. Nucleic acids can also comprise a mixture of natural and non-natural internucleoside linkages. In some embodiments, nucleic acids comprise a one type of polynucleotides or a mixture of two or more different types of polynucleotides.

The term “primer” and related terms used herein refers to an oligonucleotide, either natural or synthetic, that is capable of hybridizing with a DNA and/or RNA polynucleotide template to form a duplex molecule. Primers may have any length, but typically range from 4-50 nucleotides. A typical primer comprises a 5′ end and 3′ end. The 3′ end of the primer can include a 3′ OH moiety which serves as a nucleotide polymerization initiation site in a polymerase-mediated primer extension reaction. Alternatively, the 3′ end of the primer can lack a 3′ OH moiety, or can include a terminal 3′ blocking group that inhibits nucleotide polymerization in a polymerase-mediated reaction. Any one nucleotide, or more than one nucleotide, along the length of the primer can be labeled with a detectable reporter moiety. A primer can be in solution (e.g., a soluble primer) or can be immobilized to a support (e.g., a capture primer).

The term “template nucleic acid”, “template polynucleotide”, “target nucleic acid” “target polynucleotide”, “template strand” and other variations refer to a nucleic acid strand that serves as the basis nucleic acid molecule for generating a complementary nucleic acid strand.

The template nucleic acid can be single-stranded or double-stranded, or the template nucleic acid can have single-stranded or double-stranded portions. The sequence of the template nucleic acid can be partially or wholly complementary to the sequence of the complementary strand. The template nucleic acid can be obtained from a naturally-occurring source, recombinant form, or chemically synthesized to include any type of nucleic acid analog. The template nucleic acid can be linear, circular, or other forms. The template nucleic acids can include an insert region having an insert sequence, which is also referred to herein as a sequence of interest. The template nucleic acids can also include at least one adaptor sequence. The template nucleic acid can be a concatemer having two or tandem copies of a sequence of interest and at least one adaptor sequence. The insert region can be isolated in any form, including chromosomal, genomic, organellar (e.g., mitochondrial, chloroplast or ribosomal), recombinant molecules, cloned, amplified, cDNA, RNA such as precursor mRNA or mRNA, oligonucleotides, whole genomic DNA, obtained from fresh frozen paraffin embedded tissue, needle biopsies, cell free circulating DNA, or any type of nucleic acid library. The insert region can be isolated from any source including from organisms such as prokaryotes, eukaryotes (e.g., humans, plants and animals), fungus, viruses cells, tissues, normal or diseased cells or tissues, body fluids including blood, urine, serum, lymph, tumor, saliva, anal and vaginal secretions, amniotic samples, perspiration, semen, environmental samples, biofilms, culture samples, or synthesized nucleic acid molecules prepared using recombinant molecular biology or chemical synthesis methods. The insert region can be isolated from any organ, including head, neck, brain, breast, ovary, cervix, colon, rectum, endometrium, gallbladder, intestines, bladder, prostate, testicles, liver, lung, kidney, esophagus, pancreas, thyroid, pituitary, thymus, skin, heart, larynx, or other organs. The insert region can be isolated from a plurality of cells, or from single cells. The template nucleic acid can be subjected to nucleic acid analysis, including sequencing and composition analysis.

When used in reference to nucleic acid molecules, the terms “hybridize” or “hybridizing” or “hybridization” or other related terms refers to hydrogen bonding between two different nucleic acids to form a duplex nucleic acid. Hybridization also includes hydrogen bonding between two different regions of a single nucleic acid molecule to form a self-hybridizing molecule having a duplex region. Hybridization can comprise Watson-Crick or Hoogstein binding to form a duplex double-stranded nucleic acid, or a double-stranded region within a nucleic acid molecule. The double-stranded nucleic acid, or the two different regions of a single nucleic acid, may be wholly complementary, or partially complementary. Complementary nucleic acid strands need not hybridize with each other across their entire length. The complementary base pairing can be the standard A-T or C-G base pairing, or can be other forms of base-pairing interactions. Duplex nucleic acids can include mismatched base-paired nucleotides.

The term “nucleotides” and related terms refers to a molecule comprising an aromatic base, a five carbon sugar (e.g., ribose or deoxyribose), and at least one phosphate group.

Canonical or non-canonical nucleotides are consistent with use of the term. The phosphate in some embodiments comprises a monophosphate, diphosphate, or triphosphate, or corresponding phosphate analog. In some embodiments, the nucleotide comprises 1, 2, 3, 4, 5, 6, 7, 8, 9 or 10 phosphate groups. The term “nucleoside” refers to a molecule comprising an aromatic base and a sugar.

Nucleotides (and nucleosides) typically comprise a hetero cyclic base including substituted or unsubstituted nitrogen-containing parent heteroaromatic ring which are commonly found in nucleic acids, including naturally-occurring, substituted, modified, or engineered variants, or analogs of the same. The base of a nucleotide (or nucleoside) is capable of forming Watson-Crick and/or Hoogstein hydrogen bonds with an appropriate complementary base. Exemplary bases include, but are not limited to, purines and pyrimidines such as: 2-aminopurine, 2,6-diaminopurine, adenine (A), ethenoadenine, N⁶-Δ²-isopentenyladenine (6iA), N⁶-Δ²-isopentenyl-2-methylthioadenine (2ms6iA), N⁶-methyladenine, guanine (G), isoguanine, N²-dimethylguanine (dmG), 7-methylguanine (7 mG), 2-thiopyrimidine, 6-thioguanine (6sG), hypoxanthine and O⁶-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O⁴-methylthymine, uracil (U), 4-thiouracil (4sU) and 5,6-dihydrouracil (dihydrouracil; D); indoles such as nitroindole and 4-methylindole; pyrroles such as nitropyrrole; nebularine; inosines; hydroxymethylcytosines; 5-methycytosines; base (Y); as well as methylated, glycosylated, and acylated base moieties; and the like. Additional exemplary bases can be found in Fasman, 1989, in “Practical Handbook of Biochemistry and Molecular Biology”, pp. 385-394, CRC Press, Boca Raton, Fla.

Nucleotides (and nucleosides) typically comprise a sugar moiety, such as carbocyclic moiety (Ferraro and Gotor 2000 Chem. Rev. 100: 4319-48), acyclic moieties (Martinez, et al., 1999 Nucleic Acids Research 27: 1271-1274; Martinez, et al., 1997 Bioorganic & Medicinal Chemistry Letters vol. 7: 3013-3016), and other sugar moieties (Joeng, et al., 1993 J. Med. Chem. 36: 2627-2638; Kim, et al., 1993 J. Med. Chem. 36: 30-7; Eschenmosser 1999 Science 284:2118-2124; and U.S. Pat. No. 5,558,991). The sugar moiety comprises: ribosyl; 2′-deoxyribosyl; 3′-deoxyribosyl; 2′,3′-dideoxyribosyl; 2′,3′-didehydrodideoxyribosyl; 2′-alkoxyribosyl; 2′-azidoribosyl; 2′-aminoribosyl; 2′-fluororibosyl; 2′-mercaptoriboxy1; 2′-alkylthioribosyl; 3′-alkoxyribosyl; 3′-azidoribosyl; 3′-aminoribosyl; 3′-fluororibosyl; 3′-mercaptoriboxyl; 3′-alkylthioribosyl carbocyclic; acyclic or other modified sugars.

In some embodiments, nucleotides comprise a chain of one, two or three phosphorus atoms where the chain is typically attached to the 5′ carbon of the sugar moiety via an ester or phosphoramide linkage. In some embodiments, the nucleotide is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

When used in reference to nucleic acids, the terms “extend”, “extending”, “extension” and other variants, refers to incorporation of one or more nucleotides into a nucleic acid molecule. Nucleotide incorporation comprises polymerization of one or more nucleotides into the terminal 3′ OH end of a nucleic acid strand, resulting in extension of the nucleic acid strand. Nucleotide incorporation can be conducted with natural nucleotides and/or nucleotide analogs. Typically, but not necessarily, nucleotide incorporation occurs in a template-dependent fashion. Any suitable method of extending a nucleic acid molecule may be used, including primer extension catalyzed by a DNA polymerase or RNA polymerase.

The term “reporter moiety”, “reporter moieties” or related terms refers to a compound that generates, or causes to generate, a detectable signal. A reporter moiety is sometimes called a “label”. Any suitable reporter moiety may be used, including luminescent, photoluminescent, electroluminescent, bioluminescent, chemiluminescent, fluorescent, phosphorescent, chromophore, radioisotope, electrochemical, mass spectrometry, Raman, hapten, affinity tag, atom, or an enzyme. A reporter moiety generates a detectable signal resulting from a chemical or physical change (e.g., heat, light, electrical, pH, salt concentration, enzymatic activity, or proximity events). A proximity event includes two reporter moieties approaching each other, or associating with each other, or binding each other. It is well known to one skilled in the art to select reporter moieties so that each absorbs excitation radiation and/or emits fluorescence at a wavelength distinguishable from the other reporter moieties to permit monitoring the presence of different reporter moieties in the same reaction or in different reactions. Two or more different reporter moieties can be selected having spectrally distinct emission profiles, or having minimal overlapping spectral emission profiles. Reporter moieties can be linked (e.g., operably linked) to nucleotides, nucleosides, nucleic acids, enzymes (e.g., polymerases or reverse transcriptases), or support (e.g., surfaces).

A reporter moiety (or label) comprises a fluorescent label or a fluorophore. Exemplary fluorescent moieties which may serve as fluorescent labels or fluorophores include, but are not limited to fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY™ and derivatives such as BODIPY™ FL C3-SE, BODIPY™ 530/550 C3, BODIPY™ 530/550C3-SE, BODIPY™ 530/550 C3 hydrazide, BODIPY™ 493/503 C3 hydrazide, BODIPY™ FL C3 hydrazide, BODIPY™ FL IA, BODIPY™ 530/551 IA, Br-BODIPY™ 493/503, Cascade Blue® and derivatives such as Cascade® Blue acetyl azide, Cascade Blue® cadaverine, Cascade Blue® ethylenediamine, Cascade Blue® hydrazide, Lucifer Yellow and derivatives such as Lucifer Yellow iodoacetamide, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor® dyes, DyLight® dyes, Atto dyes, LightCycler® Red dyes, CAL Flour dyes, JOE and derivatives thereof, Oregon Green™ dyes, WellRED dyes, IRD dyes, phycoerythrin and phycobilin dyes, Malachite green, stilbene, DEG dyes, NR dyes, near-infrared dyes and others known in the art such as those described in Haugland, Molecular Probes Handbook, (Eugene, Oreg.) 6th Edition; Lakowicz, Principles of Fluorescence Spectroscopy, 2nd Ed., Plenum Press New York (1999), or Hermanson, Bioconjugate Techniques, 2nd Edition, or derivatives thereof, or any combination thereof.

Cyanine dyes may exist in either sulfonated or non-sulfonated forms, and consist of two indolenin, benzo-indolium, pyridium, thiozolium, and/or quinolinium groups separated by a polymethine bridge between two nitrogen atoms. Commercially available cyanine fluorophores include, for example, Cy3, (which may comprise 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-1,3-dihydro-2H -indol-2-ylidene}prop-1-en-1-yl)-3,3-dimethyl-3H-indolium or 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-5-sulfo -1,3-dihydro-2H-indol-2-ylidene}prop-1-en-1-yl)-3,3-dimethyl-3H-indolium-5-sulfonate), Cy5 (which may comprise 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-indolin-2-ylidene)penta-1,3-dien-1-yl)-3,3-dimethyl-3H-indol-1-ium or 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-sulfoindolin -2-ylidene)penta-1,3-dien-1-yl)-3,3-dimethyl-3H-indol-1-ium-5-sulfonate), and Cy7 (which may comprise 1-(5-carboxypentyl)-2-[(1E,3E,5E,7Z)-7-(1-ethyl-1,3-dihydro-2H-indol-2-ylidene)hepta-1,3,5-trien-1-yl]-3H-indolium or 1-(5-carboxypenty1)-2-[(1E,3E,5E,7Z)-7-(1-ethyl-5-sulfo-1,3-dihydro-2H-indol-2-ylidene)hepta-1,3,5-trien-1-yl]-3H-indolium-5-sulfonate), where “Cy” stands for ‘cyanine’, and the first digit identifies the number of carbon atoms between two indolenine groups. Cy2 which is an oxazole derivative rather than indolenin, and the benzo-derivatized Cy3.5, Cy5.5 and Cy7.5 are exceptions to this rule.

In some embodiments, the reporter moiety can be a fluorescence resonance energy transfer (FRET) pair, such that multiple classifications can be performed under a single excitation and imaging step. As used herein, FRET may comprise excitation exchange (Forster) transfers, or electron-exchange (Dexter) transfers.

The terms “linked”, “joined”, “attached”, and variants thereof comprise any type of fusion, bond, adherence or association between any combination of compounds or molecules that is of sufficient stability to withstand use in the particular procedure. The procedure can include but are not limited to: nucleotide transient-binding; nucleotide incorporation; de-blocking; washing; removing; flowing; detecting; imaging and/or identifying. Such linkage can comprise, for example, covalent, ionic, hydrogen, dipole-dipole, hydrophilic, hydrophobic, or affinity bonding, bonds or associations involving van der Waals forces, mechanical bonding, and the like. In some embodiments, such linkage occurs intramolecularly, for example linking together the ends of a single-stranded or double-stranded linear nucleic acid molecule to form a circular molecule. In some embodiments, such linkage can occur between a combination of different molecules, or between a molecule and a non-molecule, including but not limited to: linkage between a nucleic acid molecule and a solid surface; linkage between a protein and a detectable reporter moiety; linkage between a nucleotide and detectable reporter moiety; and the like. Some examples of linkages can be found, for example, in Hermanson, G., “Bioconjugate Techniques”, Second Edition (2008); Aslam, M., Dent, A., “Bioconjugation: Protein Coupling Techniques for the Biomedical Sciences”, London: Macmillan (1998); Aslam, M., Dent, A., “Bioconjugation: Protein Coupling Techniques for the Biomedical Sciences”, London: Macmillan (1998).

The term “operably linked” and “operably joined” or related terms as used herein refers to juxtaposition of components. The juxtapositioned components can be linked together covalently. For example, two nucleic acid components can be enzymatically ligated together where the linkage that joins together the two components comprises phosphodiester linkage. A first and second nucleic acid component can be linked together, where the first nucleic acid component can confer a function on a second nucleic acid component. For example, linkage between a primer binding sequence and a sequence of interest forms a nucleic acid library molecule having a portion that can bind to a primer. In another example, a transgene (e.g., a nucleic acid encoding a polypeptide or a nucleic acid sequence of interest) can be ligated to a vector where the linkage permits expression or functioning of the transgene sequence contained in the vector. In some embodiments, a transgene is operably linked to a host cell regulatory sequence (e.g., a promoter sequence) that affects expression of the transgene. In some embodiments, the vector comprises at least one host cell regulatory sequence, including a promoter sequence, enhancer, transcription and/or translation initiation sequence, transcription and/or translation termination sequence, polypeptide secretion signal sequences, and the like. In some embodiments, the host cell regulatory sequence controls expression of the level, timing and/or location of the transgene. In some cases, the components can be linked together non-covalently. The person of ordinary skill in the art will also appreciate that components need not be directly, physically linked to be operably linked.

The term “adaptor” and related terms refers to oligonucleotides that can be operably linked (appended) to a target polynucleotide, where the adaptor confers a function to the co-joined adaptor-target molecule. Adaptors can comprise DNA, RNA, chimeric DNA/RNA, or analogs thereof. Adaptors can include at least one ribonucleoside residue. Adaptors can be single-stranded, double-stranded, or have single-stranded and/or double-stranded portions. Adaptors can be configured to be linear, stem-looped, hairpin, or Y-shaped forms. Adaptors can be any length, including 4-100 nucleotides or longer. Adaptors can have blunt ends, overhang ends, or a combination of both. Overhang ends include 5′ overhang and 3′ overhang ends. The 5′ end of a single-stranded adaptor, or one strand of a double-stranded adaptor, can have a 5′ phosphate group or lack a 5′ phosphate group. Adaptors can include a 5′ tail that does not hybridize to a target polynucleotide (e.g., tailed adaptor), or adaptors can be non-tailed. An adaptor can include a sequence that is complementary to at least a portion of a primer, such as an amplification primer, a sequencing primer, or a capture primer (e.g., soluble or immobilized capture primers) as described herein. Adaptors can include a random sequence or degenerate sequence. Adaptors can include a random sequence (e.g., NNN) or can lack a random sequence. Adaptors can include at least one inosine residue. Adaptors can include at least one phosphorothioate, phosphorothiolate and/or phosphoramidate linkage. Adaptors can include a barcode sequence which can be used to distinguish polynucleotides (e.g., insert sequences) from different sample sources in a multiplex assay. Adaptors can include a unique identification sequence (e.g., unique molecular index, UMI; or a unique molecular tag) that can be used to uniquely identify a nucleic acid molecule to which the adaptor is appended. The unique identification sequence can include a random sequence (e.g., NNN) or can lack a random sequence. In some embodiments, a unique identification sequence can be used to increase error correction and accuracy, reduce the rate of false-positive variant calls and/or increase sensitivity of variant detection. Adaptors can include at least one restriction enzyme recognition sequence, including any one or any combination of two or more selected from a group consisting of type I, type II, type III, type IV, type Hs or type IIB.

The term “universal sequence”, “universal adaptor sequences” and related terms refers to a sequence in a nucleic acid molecule that is common among two or more polynucleotide molecules. For example, adaptors having the same universal sequence can be joined to a plurality of polynucleotides so that the population of co-joined molecules carry the same universal adaptor sequence. Examples of universal adaptor sequences include an amplification primer sequence, a sequencing primer sequence or a capture primer sequence (e.g., soluble or support-immobilized capture primers).

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### Sequencing Systems

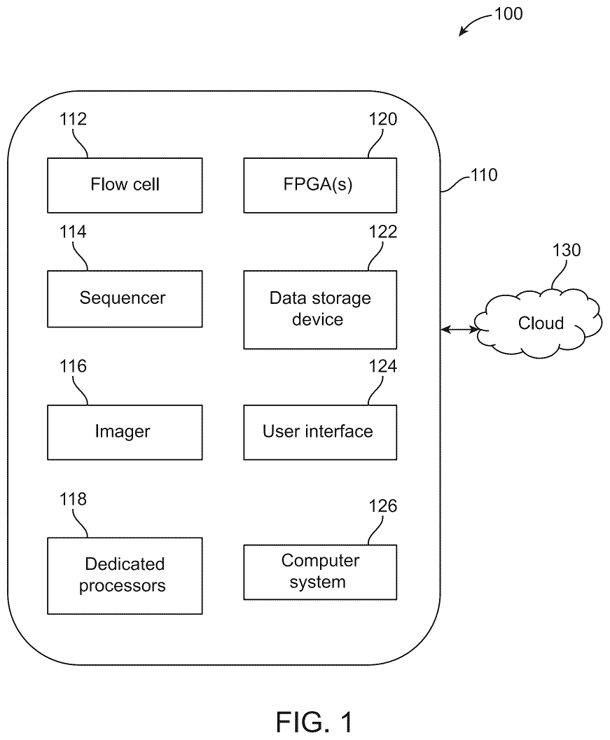

FIG. 1 illustrates a block diagram of a computer-implemented system 100 for performing sequencing and sequencing analysis, according to one or more embodiments disclosed herein. The system 100 has a sequencing system 110 that includes a flow cell device 112, a sequencer 114, an imager 116, data storage 122, and a user interface 124. The sequencing system 110 may optionally be connected to a cloud 130 (e.g., coupled to a server, compute device, database, etc.). The sequencing system 110 may include one or more of dedicated processors 118, an integrated circuit (e.g., a Field-Programmable Gate Array(s) (FPGAs)) 120, and a computer system 126.

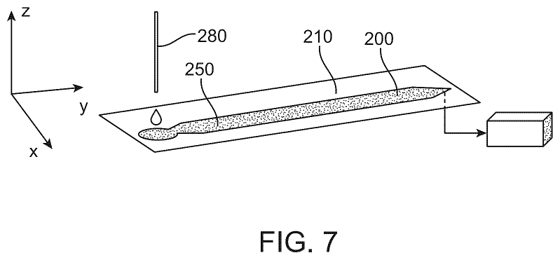

In some embodiments, the flow cell device 112 is configured to capture DNA fragments and form DNA sequences for base-calling from imaging of the flow cell device 112 by the imager 116. The flow cell 112 can include a support as described herein with respect to FIG. 7. The support can be a solid support. The support can include a surface coating thereon as disclosed herein. The surface coating can be a polymer coating as disclosed herein. The surface coating can be disposed on a surface of the one or more channels of the flow cell device 112. A different or identical surface can be placed on a surface of the inlet of the flow cell device 112.

In some embodiments, the flow cell device 112 can include a plurality of tiles (e.g., portions, locations, areas, sections, etc.) thereon configured to be imaged by the imager 116, and each tile may be separated into a plurality of subtiles. In some embodiments, the subtiles may be organized in a grid. Each subtile can include a plurality of clusters or polonies (e.g., a collection of DNA molecules such as the concatemer template molecules disclosed herein) thereon. In some embodiments, the flow cell device 112 may include a number of tiles in a range of about 1 tile to about 2000 tiles, about 100 tile to about 1500 tiles, or about 200 tile to about 500 tiles, inclusive of all ranges and subranges therebetween. In some embodiments, each tile may be divided into a number of subtiles in a range of about 2 subtiles to about 200 subtiles, about 10 subtiles to about 100 subtiles, or about 20 subtiles to about 50 subtiles, inclusive of all ranges and subranges therebetween. In some embodiments, the subtiles may be organized in a grid that may have M by N subtiles. As a nonlimiting example, the flow cell device 112 can have 424 tiles, and each tile can be divided into a 6×9 grid, therefore including 54 subtiles. In some embodiments, the imager 116 may be configured to obtain one or more images (hereinafter, “flow cell image(s)”) of the plurality of tiles, a subset of the plurality of tiles, and/or a subset of the plurality of subtiles. The flow cell image(s) as disclosed herein can include an image including signals (e.g., fluorescence levels) of the plurality of clusters or polonies. The flow cell image can include one or more tiles of signals or one or more subtiles of signals. In some embodiments, a flow cell image can be an image that includes all the tiles and approximately all signals thereon. The flow cell image can be acquired from a channel during (i) an imaging cycle or (ii) a sequencing cycle using the imager 116. In some embodiments, each tile may include millions of polonies or clusters. As a nonlimiting example, a tile can include about 1 to 10 million of clusters or polonies. Each polony can be a collection of many copies of DNA molecules.

More details of the flow cell device 112 and its functional and structural elements are disclosed herein in relation to figures, e.g., FIG. 7.

The sequencer 114 may be configured to flow mixtures of reagents onto the flow cell. Such mixtures of reagents include nucleotide mixtures, polymerases, reagents to add or cleave blockers from the nucleotides in between nucleotide addition steps, and perform other steps for the formation of the DNA molecules suitable for sequencing applications on the flow cell 112. The nucleotides may have fluorescent elements (also referred to as “labels” or “moieties”) attached that emit light or energy at a wavelength that indicates the type of nucleotide. Each type of fluorescent element may correspond to a particular nucleotide base (e.g., A, G, C, T). The fluorescent elements may emit light in visible wavelengths. In some embodiments, the sequencer 114 and the flow cell device 112 may be configured to perform various sequencing methods disclosed herein or known in the art, for example, sequencing-by-avidite, sequencing by binding or sequencing by synthesis.

For example, each nucleotide base may be assigned a color. Different types of nucleotides can have different colors. Adenine(A) may be red, cytosine(C) may be blue, guanine(G) may be green, and thymine(T) may be yellow, for example. The color or wavelength of the fluorescent element for each nucleotide may be selected so that the nucleotides are distinguishable from one another based on the wavelengths of light emitted by the fluorescent elements.

The imager 116 may be configured to capture images of the flow cell 112 after each flowing step. In an embodiment, the imager 116 may include a camera configured to capture digital images, such as a CMOS or a CCD camera. The camera may be configured to capture images at the wavelengths of the fluorescent elements bound to the nucleotides. The images can be called flow cell images.

In some embodiments, the imager 116 can include one or more optical systems disclose herein. The optical system(s) can be configured to capture optical signals from the flow cell and generate corresponding digital images thereof. The digital images can then be used for base calling. In some embodiments, the optical system and/or the flow cell 112 may be coupled to one or more moving mechanisms configured to position the flow cell 112 relative to the imager 116. In some embodiments, the flow cell 112 and/or the sequencer 114 may be coupled to one or more moving mechanisms to position the flow cell 112 relative to one or more dispensers of the sequencer. In some embodiments, the sequencer 114 may include a nest bank configured to receive and hold one or more flow cells 112 during a sequencing cycle. In some embodiments, the moving mechanism may move the flow cell 112 from the nest bank to the imager 116 for an imaging cycle. In some embodiments, the flow cell and/or a portion of the nest bank may transition to a first locked (e.g., registered) configuration when the flow cell 112 is disposed on or near the nest bank such that the flow cell 112 is in fluid communication with the nest bank. In some embodiments, the flow cell 112 may be configured to transition from the locked configuration to an unlocked configuration to be moved to the imager 116. In some embodiments, the flow cell 112 may transition from unlocked configuration to a second locked configuration such that the flow cell 112 can be positioned relative to the imager 116 (e.g., without mechanical, fluid, or thermal disturbances). In some embodiments, multiple flow cells 112 may be in the sequencing system 110 simultaneously. For example, a first flow cell may undergo an imaging cycle while a second flow cell undergoes a sequencing cycle. In this way, the sequencing system 110 may increase efficiency of sequencing and have a high throughput of samples. In some embodiments, the imager 116 (e.g., and/or any part of the optical system) may be fluidically isolated from the sequencer 114 to prevent exposure of fluid to the imager 116 and/or improve image quality. In some embodiments, the moving mechanism may include at least one of a movable arm and/or an x-y stage. In some embodiments, the processors of the sequencing system 110 (e.g., the dedicated processor 118, FPG(s) 120 and/or the CPU of the computer system 126 may each be configured to run different tasks such that simultaneous sequencing and imaging may be carried out.

In an embodiment, the images of the flow cell may be captured in groups, where each image in the group is taken at a wavelength or in a spectrum that matches or includes only one of the fluorescent elements. In another embodiment, the images may be captured as single images that captures all of the wavelengths of the fluorescent elements.

The resolution of the imager 116 controls the level of detail in the flow cell images, including pixel size. In existing systems, this resolution is very important, as it controls the accuracy with which a spot-finding algorithm identifies the polony centers. In some embodiments, the image resolution of flow cell images disclosed herein can be about 10 nanometers (nm) to 900 nm, inclusive of all ranges or subranges therebetween. In some embodiments, the image resolution of the flow cell images can be between about 10 nm to about 900 nm, about 10 nm to about 500 nm, about 10 nm to about 200 nm, about 20 nm to about 500 nm, about 20 nm to about 200 nm, or any range or subrange therebetween. One way to increase the accuracy of spot finding is to improve the resolution of the imager 116, or improve the processing performed on images taken by imager 116. Detecting polony centers in pixels other than those detected by a spot-finding algorithm can be performed. Suitable spot-finding algorithms will be known to persons of ordinary skill in the art. These methods can allow for improved accuracy in detection of polony centers without increasing the resolution of the imager 116. The resolution of the imager 116 may even be less than existing systems with comparable performance, which may reduce the cost of the sequencing system 110.

The image quality of the flow cell images can control the base calling accuracy. The imager 116 disclosed herein can increase the accuracy of base calling. Alternatively the processing performed on images taken by imager 116 can result in a better image quality, thereby increasing the accuracy of base calling using the systems disclosed herein.

After base calling is performed, a processor (e.g., dedicated processors 118, FPGA(s) 120, computer system 126, or a combination thereof) may optionally perform additional processing and/or analysis of base calling results. In some embodiments, after base calling is performed, the sequencing read(s) (processed and/or raw) can be outputted from the system to an external device (e.g., the cloud 130 and/or to a computer system 400). The sequencing read(s) herein can include a forward read (R1), a reverse read (R2), or both. The sequencing read(s) herein can be any orderly sequence of bases of A, T, C, and G.

In some embodiments, the sequencing read(s) can be communicated (e.g., directly or indirectly) to the computer system 126 for subsequent analysis such as adaptor trimming, or phasing for example.

These sequencing analysis methods, including primary analysis and/or secondary analysis, can be advantageously performed in parallel in the computer system 126, without interference with or delay of existing sequencing workflow of the system 100. The results of sequencing analysis can be made available for generating sequencing results for users. Some or all operations of the sequencing process can be advantageously performed by the FPGA(s) and data can be communicated between the CPU(s) and FPGA(s) to reduce the total operational time from methods operating without the FPGA(s).

The operations or actions disclosed herein may be performed by the dedicated processors 118, the FPGA(s) 120, the computing system 126, or a combination thereof. One or more operations or actions (e.g., methods) disclosed herein may be performed by the dedicated processors 118, the FPGA(s) 120, the computing system 126, or a combination thereof. In some embodiments, which operations or actions are to be performed by performed by the dedicated processors 118, the FPGA(s) 120, the computing system 126, or their combinations can be determined based on one or more of: a computation time for the specific operation(s), the complexity of computation in the specific operation(s), the need for data transmission between the hardware devices, and/or their combinations.

The computing system 126 can include one or more general purpose computers that provide interfaces to run a variety of program in an operating system, such as Windows™ or Linux™. Such an operating system typically provides great flexibility to a user.

In some embodiments, the dedicated processors 118 may not be general-purpose processors, but instead custom processors with specific hardware or instructions for performing method steps. Dedicated processors 118 may directly run specific software without an operating system. The lack of an operating system reduces overhead, at the cost of the flexibility in what the dedicated processors 118 may perform. A dedicated processor 118 may make use of a custom programming language, which may be designed to operate more efficiently than the software run on general-purpose computers. This may increase the speed at which the steps are performed and allow for real time processing.

In some embodiments, the FPGA(s) 120 may be configured to perform operations of the sequencing analysis methods herein. An FPGA is programmed as hardware that will only perform a specific task. A special programming language may be used to transform software steps into hardware componentry. Once an FPGA is programmed, the hardware directly processes digital data that is provided to it without running software. The FPGA instead uses logic gates and registers to process the digital data. Because there is no overhead required for an operating system, an FPGA generally processes data faster than a general-purpose computer. Similar to dedicated processors 118, this is at the cost of flexibility.

The lack of software overhead may also allow an FPGA 120 to operate faster than a dedicated processor 118, although this can depend on the exact processing to be performed and the specific FPGA 120 and dedicated processor 118.

A group of FPGA(s) 120 may be configured to perform processing steps in parallel. For example, a number of FPGA(s) 120 may be configured to perform a processing step for an image, a set of images, a subtile, or a select region in one or more images. In some embodiments, each FPGA 120 may perform a respective step or substep of the processing steps at the same time, reducing the time needed to process data. This may allow the processing steps to be completed in real time or near real-time. Further discussion of the use of FPGAs is provided below.

Performing the processing steps in real time may allow the system 100 to use less memory, as the data may be processed as it is received rather than stored for subsequent analysis. This provides advantages over conventional systems, which may store the data before the data is processed, which may require more memory and/or accessing and communication with a computer system located in the cloud 130. In some embodiments, the data storage device 122 is used to store information used in or obtained from sequencing analysis. For example, the DNA sequences determined after adaptor trimming may be stored in the data storage 122. Compressed and/or uncompressed sequencing data may be stored in the data storage. The FASTQ file may also be stored in the data storage 122.

The user interface 124 may be used by a user to operate the sequencing system or access data stored in the data storage 122 or the computer system 126.

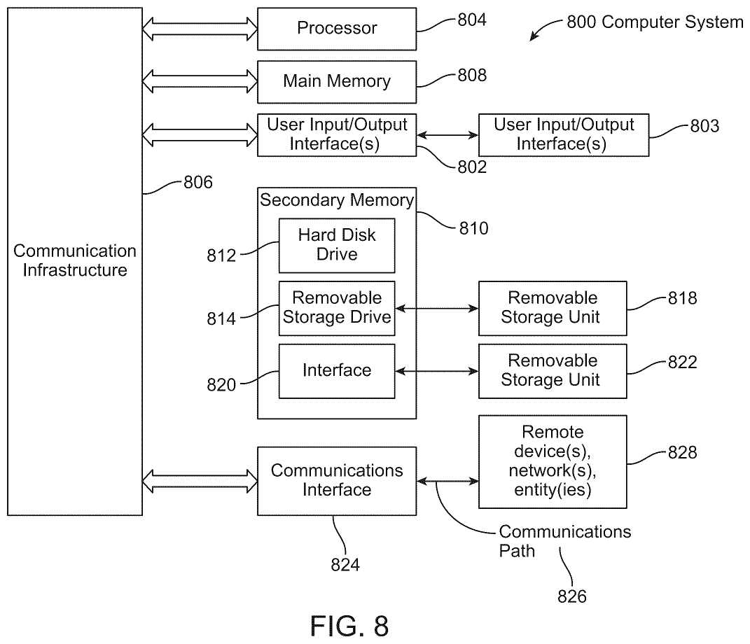

The computer system 126 may control the general operation of the sequencing system and may be coupled to the user interface 124. In some embodiments, the computer system 126 may perform one or more steps in sequencing analysis, such as base calling, adaptor trimming, demultiplexing, phasing etc. In some embodiments, the computer system 126 may be structurally and/or functionally similar to the computer system 800, as described in more detail in FIG. 8. The computer system 126 may include a memory configured to store information regarding the operation of the sequencing system 110, such as, for example, configuration information, instructions for operating the sequencing system 110, or user information. The computer system 126 may be configured to pass information between the sequencing system 110 and the cloud 130. For example, the computer system 126 may be configured to receive base calling results from the dedicated processors 118 and/or FPGA(s) and send the base calling results to the cloud 130 for storage and/or further analysis.

As discussed above, the sequencing system 110 may have dedicated processors 118, FPGA(s) 120, or the computer system 126. The sequencing system 110 may use one, two, or all of these elements to accomplish necessary processing described above. In some embodiments, when these elements are present together, the processing tasks are split between them. The FPGA(s) 120 may be used to perform some portion or all of sequencing analysis operations, while the computer system 126 may perform other processing functions for the sequencing system 110. The distribution of processing across the dedicated processor(s) 118, the FPGA(s) 120, and/or general purpose processors (e.g., in the computer system 126) can enable parallel processing and/or increase efficiency of processing steps. For example, complex processing steps may be allocated to the dedicated processor(s) 118 and/or FPGA(s) 120 while processing for general operation of the system 110 is carried out by the computer system 126.

Those skilled in the art understand that various combinations of these elements can allow various system embodiments that balance efficiency and speed of processing with cost of processing elements. The cloud 130 may be a network, server, remote storage, or some other remote computing system separate from the sequencing system 110. The connection to cloud 130 may allow access to data stored externally to the sequencing system 110 or allow for updating of software in the sequencing system 110.

### Flow Cell Devices

Disclosed herein, in some embodiments, are flow cell devices and systems that can be employed for performing or facilitating DNA sequencing analysis. Flow cell devices herein can be used to immobilize template nucleic acid molecules derived from biological samples and then introduce a repetitive flow of sequencing reagents (e.g., sequencing by binding, sequencing by synthesis, and/or sequencing by avidite) to attach labeled nucleotides or labeled multivalent molecules to specific positions in the template sequences. A series of label signals are detected and decoded to reveal the nucleotide sequences of the template molecules, e.g., immobilized and/or amplified nucleic acid template molecules attached to a surface of the flow cell.

FIG. 7 shows an exemplary embodiment of a flow cell device 200. The flow cell device 200 may include a support (e.g., as surface) having one or more substrates disposed thereon, a number of channels (not shown), an inlet (not shown), and outlet (not shown). In some embodiments, liquid (280) is dispensed from a dispenser to the flow cell device 200.

In some embodiments, the support 210 of the flow cell device 200 disclosed herein can be configured to define or receive the one or more channels and/or one or more substrates. In some embodiments, the support 210 can be solid, i.e. firm and stable in shape. At least part of the support 210 can be transparent so that light transmitting from a light source of the imager (116 in FIG. 1) can travel through the transparent portion of the support 210 and reach the samples located on the flow cell device 200.

The support 210 can include or receive one or more substrates. When the flow cell device 200 is placed in the sequencing system (e.g., sequencing system 110) for imaging, the top substrate can be closer to the camera of the imager (e.g., imager 116), along the z direction, than the bottom substrate. The bottom substrate can be closer to an x-y stage of the sequencing system 110 for holding and supporting the flow cell 200 during sequencing than the top substrate.

In some embodiments, the flow cell device 200 can further include a middle substrate in between the top and bottom substrate.

Each substrate can have a predetermined thickness. In some embodiments, any or all of the substrates can have different thickness. In some embodiments, each substrate can have a uniform thickness along the z direction. In some embodiments, each substrate can have a uniform thickness along the z direction in at least a portion of the substrate. For example, the portion with uniform thickness can encompass the channel(s) or the imaging areas of the flow cell device.

In some embodiments, the top substrate and/or the bottom substrate may have a first thickness and the middle substrate may have a second thickness smaller than the first thickness. In some embodiments, the top and/or bottom substrate can have a thickness of about 0.2 mm to about 5 mm, inclusive of all ranges and subranges therebetween. In some embodiments, the top and/or bottom substrate can have a thickness of about 0.6 mm to about 3 mm, inclusive of all ranges and subranges therebetween. In some embodiments, the top and/or bottom substrate can have a thickness of about 0.8 mm to about 2 mm, inclusive of all ranges and subranges therebetween. In some embodiments, the top and/or bottom substrate can have a thickness of about 0.8 mm to about 1.5 mm, inclusive of all ranges and subranges therebetween.

In some embodiments, the middle substrate can have a thickness of about 40 um to 200 um, inclusive of all ranges and subranges therebetween. In some embodiments, the middle substrate can have a thickness of about 40 um to 150 um, inclusive of all ranges and subranges therebetween. In some embodiments, the middle substrate can have a thickness of about 40 um to 70 um, inclusive of all ranges and subranges therebetween.

In some embodiments, the middle substrate can have a thickness of about 80 um to 120 um, inclusive of all ranges and subranges therebetween.

In some embodiments, the substrate(s) can form an elongate shape extending along the y axis on a surface of the support 210 on which they are disposed. In some embodiments, the substrate(s) can have various shapes such as rectangular, square, oval, etc.

In some embodiments, the one or more substrates can be planar or substantially planar. In some embodiments, the one or more substrates contains no curvature perceivable to naked eyes, so that the one or more substrates can have planar surfaces. However, the substrates do not have to be planar in certain embodiments. Alternatively, a part or the entirety of one or more substrates can be curved.

In some embodiments, the support 210 or the one or more substrates can comprise glass or plastic. In some embodiments, the support or one or more substrates are all-glass or all-plastic. In some embodiments, the support or the one or more substrates can comprise a tape, such as a pressure sensitive adhesive (PSA) tape.

The substrate(s) can define one or more channels 250 of the flow cell devices 200 (e.g., extending longitudinally along a top surface of the flow cell device 200. The channels 250 can allow fluid, e.g., liquid or gas, to flow therethrough.

The gas herein can comprise one type of gas or a combination of different type of gases. In some embodiments, the gas comprises air. The gas can comprise dry air. In some embodiments, the gas comprises one or more inert gases, for example argon or nitrogen. In some embodiments, the gas comprises one or more active gases.

The reagents for sequencing described herein can include liquid. In some embodiments, the reagents can be deprived of air bubbles that are greater than a predetermined size (e.g., to improve accuracy and/or repeatability of reactions conducted in the flow cell device, enhance the clarity of images taken by the optical systems of the disclosure and, and/or enhance the transmission of fluorescence light used during excitation). In some embodiments, a first reagent is configured to wet the first coating of the surface of the one or more channels 250. In some embodiments, the second reagent is configured to rewet the surface of the one or more channels 250 after at least partly drying the surface by the gas gap.

In some embodiments, the channels 250 can include microfluidic channels. In some embodiments, a gap or height between the top interior surface and the bottom interior surface of the substrates that defines the channels 250, along the z direction, is about 150 um, 130um, 120 um, 110 um, 100 um, 90 um, 80 um, 70 um, 60 um, 50 um, or 40 um, or any range therebetween. In some embodiments, the gap or height of the channel 250 is no more than about 100 um. In some embodiments, the gap or height of the channel 250 is no more than about 60 um, 50um, or 40 um.

In some embodiments, a length of the channel 250, along the y direction, is about 120 mm, 100 mm, 90 mm, 80 mm, 70 mm, 60 mm, 50 mm, 40 mm, or 30 mm or any range therebetween. In some embodiments, the length of the channel 250 is no more than about 100 um. In some embodiments, the length of the channel 250 is no more than about 80 mm, 75 mm, 70 mm, 65 mm, 60 mm, 55 mm, or 50 mm.

In some embodiments, a width of the channel 250, along the x direction, is about 50 mm, 40 mm, 30 mm, 25 mm, 20 mm, 10 mm, 15 mm, 8 mm, or 5 mm or any range therebetween. In some embodiments, the length of the channel 250 is no more than about 10 mm or about 7 mm. In some embodiments, the width of the channel 250 is no more than about 40 mm, 35 mm, 30 mm, 25 mm, 20 mm, or 15 mm.

In some embodiments, the distance between two adjacent channels 250 or the distance from an edge of the channel 250 to the edge of the flow cell device 200, along the x axis, is about 0.5 mm to about 15 mm. In some embodiments, the distance between two adjacent channels 250 or the distance from an edge of the channel 250 to the edge of the flow cell device, along the x axis, is about 1 mm to about 5 mm.

In some embodiments, the flow cell devices 200 can have more than one channel 250, and all the channels 250 can have a unform size and shape. In some embodiments, the flow cell devices 200 can have channels 250 of different sizes and/or shapes.

The flow cell device 200 can include one or more inlets and one or more outlets. A channel 250 can run or extend from its corresponding inlet to its corresponding outlet, thereby allowing fluidic communication from the corresponding inlet to the corresponding outlet. Sequencing reagents can be introduced to the flow cell device 200 via the inlet, flow through each of the channels 250 and interact with samples located therein, and then exit from the outlet.

The size and shape of the inlet and outlet can be customized to suit various sequencing applications.

The flow cell devices, fluidic control devices, and systems can include an open landing area onto which reagents 280 can be flowed. FIG. 7 shows flow devices with an open landing area.

In some embodiments, the open landing area can be formed at least in part by a corresponding inlet. The open landing area can be on a bottom substrate. For example, the open landing area can be formed by voids in or by extensions of or apertures in corresponding areas of the middle and top substrates. In some embodiments, each channel 250 may be coupled to a corresponding open landing area. The open landing area can be in fluidic connection with its corresponding channel(s). In some embodiments, the open landing area is sealed. In some embodiments, the open landing area is open to external or atmospheric gases. In some embodiments, the open landing area is exposed to ambient air or to such gaseous atmosphere as surrounds the instant systems and apparatus.

### Nest Banks and Movable Arms

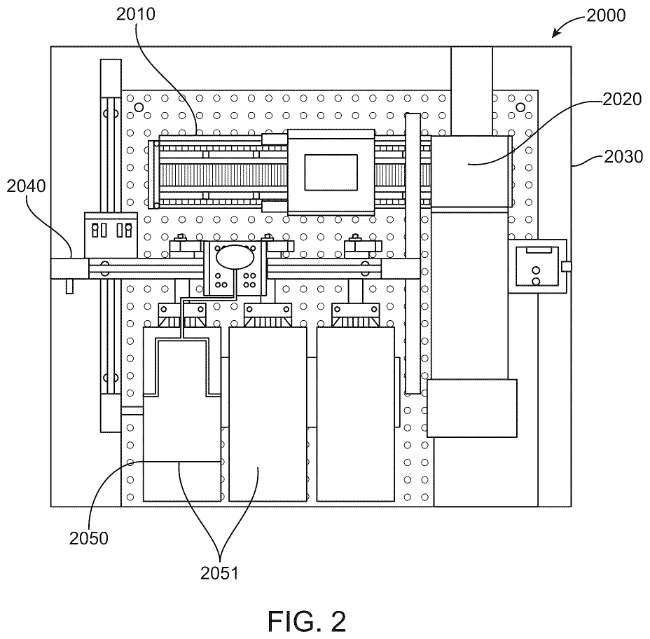

In some embodiments, the sequencing systems 2000 disclosed herein may include an optical system 2020 including an objective lens; an x-y stage 2010 configured to hold a sample to be imaged thereon and to move the sample within an x-y plane relative to the objective lens, wherein the sample is immobilized on one or more flow cell devices, as shown in FIG. 2. The sequencing system 2000 may further include a nest bank 2050 configured to provide fluidic and thermal communication to the sample when the one or more flow cell devices are coupled to the nest bank 2050; and a moving mechanism 2040 (e.g., a movable arm) configured to move the one or more flow cell devices between the x-y stage 2010 and the nest bank 2050 during a sequence run. The nest bank 2050 may include one or more identical nest modules. As used herein, “nest module” refers to one or more regions of the nest bank configured to receive a carrier 2051 and provide fluidic and thermal fluidic to communication to the carrier 2051 and/or a flow cell device disposed therein.

In some embodiments, the sequencing system 2000 comprises a housing (not shown) configured for holding one or more of the optical system, the x-y stage 2010, the nest 2050 bank, the carrier 2051, and a moving mechanism 2040 comprising a movable arm therewithin. In other words, the nest bank 2050, moving mechanism2040, and the carrier(s) 2051 can be positioned within the integral console box of the sequencing system. Alternatively, one or more of the structural elements, e.g., the nest bank and movable arm, may be positioned at least partially external to the housing of the sequencing system 2000.

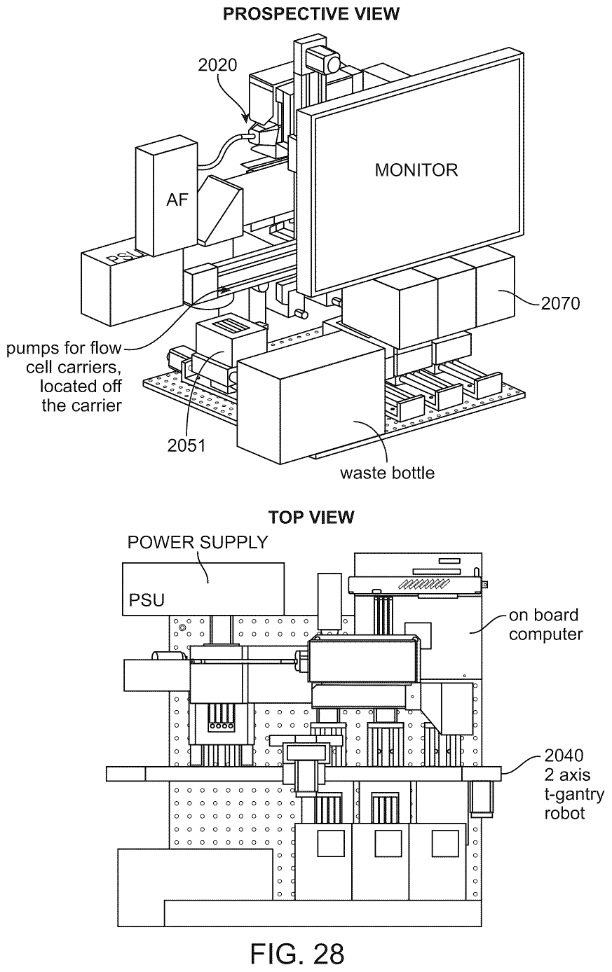

The x-y stage herein may be equivalent to the translation stage and/or the sample stage of the sequencing system. The x-y stage may be actuated automatically by an actuator, e.g., a first actuator, with a first spatial precision. Various actuators can be used herein, for example, a motor, a cam, or a gear system. In some embodiments, the x-y stage may be actuated manually (e.g., by a user). In some embodiments, the x-y stage, e.g., 2010 in FIG. 2 and FIG. 27A, includes a base and a movable stage coupled thereon. In some embodiments, the x-y stage 2010 may be actuated to move in 3 dimensional space (3D). In some embodiments, the x-y stage 2010 may move. In some embodiments, the x-y stage 2010 is actuated to move for a predetermined distance, e.g., for focusing the sample relative to the objective lens. The predetermined distance may be in 3D. In some embodiments, the predetermined distance may be along the x-y plane. In some embodiments, the predetermined distance is based on the distance between two adjacent microfluidic channels of the flow cell device. In some embodiments, the x-y stage 2010 is actuated to move from a first position (e.g., predetermined start position) to a second position (e.g., a predetermined stop position). For example, the x-y stage 2010 may be actuated to move between two different spatial positions (e.g., the start position and the stop position) so that each of the two microfluidic channels of a same flow cell device can be positioned relative to the objective lens for imaging. In some embodiments, the x-y stage 2010 is actuated to move the sample relative to the objective lens for imaging. The x-y stage 2010 can move with various speed and/or spatial precisions (e.g., ±0.1 mm, ±0.100 μm) from the predetermined start position and stop position in 3D. The time needed for moving the sample to predetermined location relative to the objective lens can be within 0.5 second to 10 seconds, inclusive of all ranges and subranges therebetween. The time needed for moving the sample to predetermined location relative to the objective lens can be within 0.5 second to 3 seconds, inclusive of all ranges and subranges therebetween. Various speeds can be used to optimize the time for moving the sample and the accuracy of moving. For example, the speed can be in a range from 0.001 millimeters (mm)/second to 2 meters/second, for example between 0.001 mm/second to 100 centimeters (cm)/second, between 0.01 mm/second and 50 cm/second or between 0.1 mm/second and 50 mm/second.

In some embodiments, the x-y stage 2010 in a sequencing system 2000 is different from the sample stage and/or translation stage of existing sequencing systems in that it the x-y stage 2010 (or nearby the x-y stage 2010) is not in fluidic communication or thermal communication with the one or more flow cell devices when the flow cell devices are immobilized on the x-y stage 2010. In existing systems, fluidic and thermal communications (e.g., fluidic or heat exchange) or connection (fluidic pathway and physical contact) may exist while the sample is immobilized on the x-y stage even while the sample is being imaged. In contrast, the sequencing systems disclosed herein 2000 lack fluidic and thermal communications (e.g., fluidic or heat exchange) or connection (fluidic pathway and physical contact) and the sample is free from fluidic and/or thermal communication or connection when the sample is on the x-y stage.

In some embodiments, the x-y stage 2010 may be coupled with a kinematic mount for accurate positioning. For example, magnetic contacts of kinematic mounts can be engaged through induction magnetism to load the flow cell device and disengaged to release the flow cell device for loading the next flow cell device. The sequencing system 2000 with the moving mechanism 2040, nest bank 2050, and x-y stage 2010 may create higher imaging reliability, manufacturing robustness, and flexibility as the imaging is completely isolated from fluidic and thermal communication when compared with existing systems. The motion of the x-y stage 2010, e.g., along the x axis, can be enabled with a cam system which decreases costs and increases speed and flexibility.

In some embodiments, the x-y stage 2010 may be coupled with one or more sensors to provide feedback of the alignment of the flow cell device relative to the x-y stage 2010 and/or the optical system. Various sensors can be used, such as visual light sensors, audio, other light sensors like infrared, pressure, and/or force sensors. The feedback of one or more sensors may be sent to a processor of the sequencing system 2000 or a processor external to the sequencing system 2000, and the feedback can be used to fine tune the positioning of the flow cell device relative to the x-y stage 2010 and/or the optical system so that it is aligned with the objective lens for imaging. For example, an audio sensor may be used to detect sound waves bounced back from the carrier 2051 to determine a distance between the carrier 2051 and the aligned imaging position (e.g., to determine a difference between the actual position of the carrier 2051 and the desired position of the carrier 2051). As another example, camera(s) may be used to detect whether the carrier is positioned at the aligned imaging position or not.

In some embodiments, the x-y stage 2010 may include a hardware processor that is different from and external to the processor of the sequencing system 2000. The hardware processor of the x-y stage 2010 may communicate with the one or more sensors such that the x-y stage 2010 does not communicate with the processor(s) of the sequencing system (e.g., the one or more dedicated processor 118, FPGA(s) 120, a processor of the computer system 126) to process the feed-back from the one or more sensors, and generate instructions for the actuators(s) (e.g., the tip-tilt device, the motor) to move the x-y stage 2010 to desired 3D locations. The movement can be with the predetermined precision (e.g., ±0.1mm, ±0.100 um). In some embodiments, the x-y stage 2010, may include a hardware processor that is different from and external to the processor of the sequencing system to enable autonomous or semi-autonomous movement of the x-y stage 2010 based on the feedback(s) of the one or more sensors.

In some embodiments, the x-y stage does not include any hardware processor external to the processor of the sequencing system. The one or more sensors may communicate with one or more of the processors of the sequencing system 2000 (e.g., the one or more dedicated processor 118, FPGA(s) 120, or a processor of the computer system 126) to process the feedback from the one or more sensors, and generate instructions for the actuators(s) (e.g., the tip-tilt device, the motor) to move the x-y stage 2010 to desired 3D locations.

In some embodiments, the motion of the x-y stage can be linear. For example, the x-y stage 2010 may move along x, y, or any linear axis in 3D. In some embodiments, the motion of the x-y stage is nonlinear, e.g., rotational in 3D.

In some embodiments, the optical system 2020 may be movable in one or more directions in 3D. In some embodiments, the optical system 2020 may be coupled with a kinematic mount for accurate positioning in 3D. In some embodiments, the optical system 2020 may be movable at a predetermined spatial precision, and a predetermined speed range. For example, the x-y stage 2010 may move linearly along the x axis and the optical system (e.g., at least the objective lens) can move linearly along the y axis, and the combination of movements of the optical system and the x-y stage 2010 can allow the sample to be positioned at a predetermined position relative to the objective lens for imaging. As another example, one of the x-y stage 2010 or the optical system may move nonlinearly, e.g., rotate about a predetermined origin, and the other one of the x-y stage 2010 or the optical system may move linearly and the combination of movements of the optical system and the x-y stage 2010 allow the sample to be positioned at a predetermined position relative to the objective lens for imaging. The x-y stage 2010 and the optical system may be moveable in many directions relative to one another to precisely position the sample relative to the objective lens.

In some embodiments, the movable arm may be actuated automatically by an actuator, e.g., a second actuator, with a second spatial precision. The first actuator, the second actuator, or both may be controlled by one or more hardware processors of the sequencing system 2000 (e.g., similar to the one or more dedicated processor 118, FPGA(s) 120, a processor of the computer system 126). The movable arm may be actuated automatically to move in three dimensions (3D). The movement of the movable arm in each of the three dimensions can be of identical or different spatial precisions. The movable arm can move with various speed and spatial precisions. In some embodiments, the speed and precision of the movable arm are determined by the user. The time needed for moving the sample from the nest bank 2050 to the x-y stage 2010 can be within 0.5 second to 10 seconds, inclusive of all ranges and subranges therebetween. The time needed for moving the sample from the nest bank 2050 to the x-y stage 2010 can be within 0.5 second to 3 seconds, inclusive of all ranges and subranges therebetween. The movable arm of the moving mechanism 2040 can move at various speeds, for example, the speed can be in a range from 1 mm/second to 2 meters/second, inclusive of all ranges and subranges therebetween. As a further example, the speed can range from 1 mm/second to 500 centimeters (cm)/second, between 10 mm/second and 50 cm/second or between 1 cm/second and 100 cm/second.

In some embodiments, the movable arm of the moving mechanism 2040 is configured to move the one or more flow cell devices between two spatial positions in 3 dimensions (e.g., between the x-y stage 2010 and the nest bank 2050) with a first spatial precision. For example, the movable arm may be configured to move a flow cell device from the nest bank 2050 to the x-y stage 2010 for imaging of a portion of the flow cell device and/or from the x-y stage 2010 back to the nest bank 2050 after imaging.

FIGS. 27A-27C show different exemplary embodiments of the sequencing system including the movable arm disclosed herein. As shown in FIGS. 27A-27C, the sequencing system may include the one or more flow cell devices in a respective flow cell carrier device 2051 disposed in a nest bank 2050 located at a first position relative to the optical system 2020 including the objective lens. The nest bank 2050 may be positioned a certain distance away from the objective lens such that when reagents are flowed over the flow cell devices, there is separation between the optical system and fluid and/or temperature changes. In some embodiments, each flow cell carrier 2051 may be coupled to a respective x-y stage 2010, and the moving mechanism 2040 may pick up a carrier 2051 (e.g., with the respective x-y stage 2010), move the carrier 2051 to a second position near the objective lens, and dispose the carrier 2051 at the second position. In some embodiments, the sequencing system may include one x-y stage 2010 positioned near the objective lens, and the moving mechanism 2040 may be configured to move a flow cell carrier 2051 onto the x-y stage 2010 for imaging.

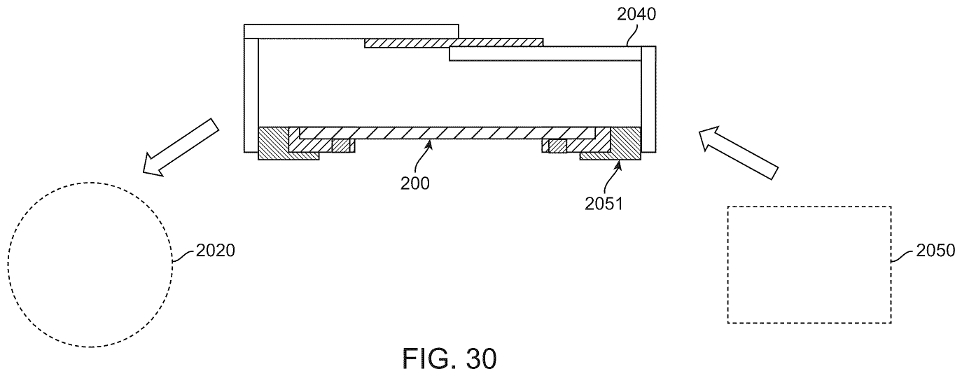

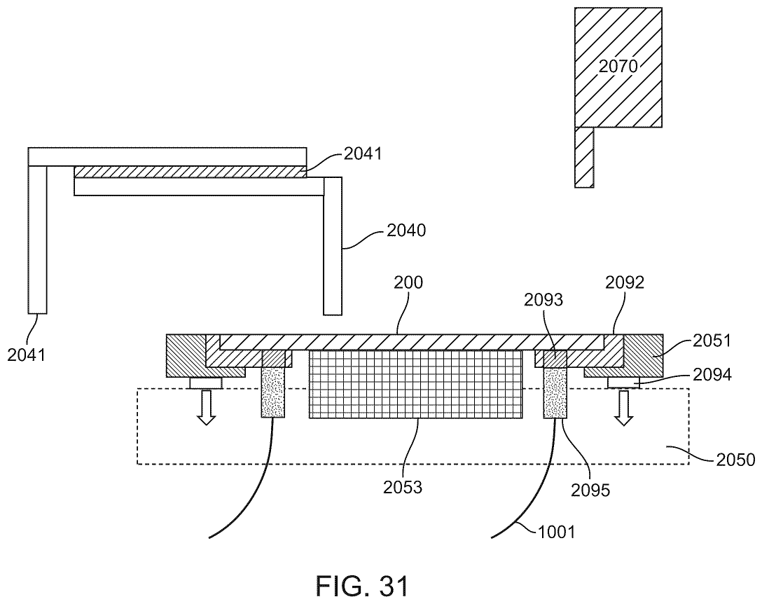

The movable arm may comprise a grabber, e.g., 2043 in FIG. 27A, that is configured to grab or otherwise hold a carrier 2051. In some embodiments, the movable arm and the carrier 2051 may be configured to transition between a coupled state and an uncoupled state, further described in FIGS. 30-31. In the coupled state, the movable arm may be securely coupled to the flow cell device and its carrier 2051. The movable arm may move the carrier securely, e.g., between the x-y stage 2010 for imaging and the nest bank 2050, when in the coupled state. The movable arm may be configured to transition to the uncoupled state, in which the carrier 2051 is removed from the movable arm when the flow cell device and its carrier 2051 are at a target location. The grabber 2043 of the movable arm may be configured to receive and/or hold the carrier 2051 when the carrier 2051 is in a decoupled position in relation to the nest bank or when the carrier is in the decoupled position in relation to the x-y stage. In some embodiments, the movable arm comprises a horizontal arm 2041 (e.g., a bar, support, track, extension, etc.) that is coupled to (e.g., mechanically supported by) a vertical arm 2042 (e.g., a bar, support, track, extension, etc.), as shown in FIG. 27A. The horizontal arm 2041, vertical arm 2042, and grabber 2043 can move in 3D relative to the housing of the sequencing system or any other reference point of the sequencing system so that the grabber 2043 can grab the flow cell carrier 2051 and move it between the nest bank 2050 and the x-y stage 2010. In some embodiments, the horizontal arm 2041 and/or the vertical arm 2042 may be positioned above the nest bank 2050, and the grabber 2043 may face downward. As shown in FIG. 27B, the movable arm of the moving mechanism 2040 may be coupled to a surface of the sequencing system on which the nest bank 2050 and x-y stage 2010 are disposed. In some embodiments, a base of the movable arm may be fastened to the surface of the sequencing system, and the movable arm 2040 may include one or more joints at which the movable arm may bend and/or rotate. In some embodiments, the movable arm may include one or more of an upper arm (e.g., a first portion), a joint, a forearm (e.g., a second portion), a wrist, and a grabber attached to the forearm, e.g., as shown in FIG. 27B. The upper arm and the forearm may be coupled to one another via a first joint, and the grabber may be coupled to the forearm via a second joint. Some or all of components of the movable arm may be individually movable relative to the housing of the sequencing system or any other reference point of the sequencing system. The movable arm in such embodiments may move in 3D with 6 degrees of freedom by combining the motion of one or more of the components of the movable arm.

FIGS. 30-31 show an exemplary embodiment of the movable arm disclosed herein.

The movable arm may include a coupled state as shown in FIG. 30, in which the movable arm is securely coupled with the flow cell device 200 and its carrier 2051. The movable arm may move the carrier 2051 securely, e.g., between the x-y stage 2010 for imaging and the nest bank 2050, when it is in a coupled state. The movable arm may include an uncoupled state, as shown in FIG. 31, in which the flow cell device 200 and its carrier 2051 are removed from the movable arm. In some embodiments, the grabber of the movable arm in the coupled state may have a width greater than the width of the grabber in the uncoupled state. For example, the grabber in the uncoupled state may have a neutral position having a width that is smaller than a width of the carrier 2051 and may be configured to extend to accommodate a width of the carrier 2051 such that the grabber exerts a pinching force on the carrier 2051 to hold secure the carrier 2051 between one or more extensions (e.g., fingers, claws) of the grabber.

In some embodiments, the movable arm comprises one or more arm elements that are movable relative to each other. As shown in FIGS. 30-31, the horizontal arm 2041 can support two different forearms 2041 for moving at least along the horizontal plane (e.g., x-y plane). In some embodiments, the two different forearms 2041 may also move along z axis that is perpendicular to the horizontal plane.

In some embodiments, the grabber 2043 can use various mechanisms for grabbing or otherwise holding the carrier 2051 and moving the carrier along with it. For example, the grabber 2043 may include fingers as shown in FIGS. 27A-27B. As another example, the grabber 2043 may lack any finger-like structures. In some embodiments, the grabber may use frictional forces, magnetic forces, electromagnetic forces for grabbing or otherwise holding the carrier 2051 so that it moves the carrier along with it.

In some embodiments, the grabber is movably attached to the horizontal or vertical arm. In some embodiments, the grabber is configured to move in 3D relative to the housing of the sequencing system or any other reference point of the sequencing system.

In some embodiments, the moving mechanism 2040 comprises a plurality of tracks, e.g., as shown in FIG. 27C. Each track may connect a carrier 2051 coupled to the nest bank 2050 to the x-y stage 2010. In some embodiments, the carrier 2051 may be actuated by a motor or otherwise an actuator to move along the track between the x-y stage 2010 and the nest bank 2050. Comparing with embodiments in FIGS. 27A-27B, the tracks allow the flow cell device to move with less flexibility in 3D but may be simpler, less prone to variability in its movements, or more compact because of the predetermined layout of traveling tracks.

In some embodiments, the moving mechanism 2040 comprises one or more belt conveyors that may function similarly as the tracks to move the carriers between the x-y stage 2010 and the nest bank 2050.

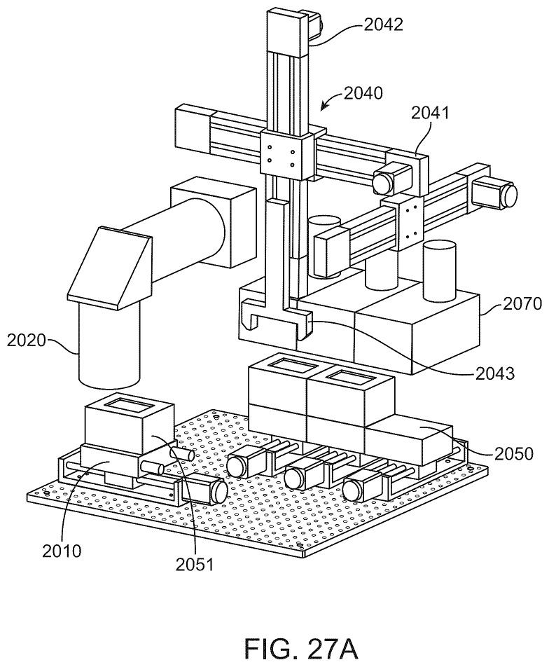

FIG. 25 shows an exemplary layout of the moving mechanism 2040, nest bank 2050, and the optical system (including the objective lens) with respect to the carrier(s) 2051. In some embodiments, the carrier 2051 may have at least two docking positions, the fluidics position 2503 and the imaging position 2502. There may be multiple flow cell carriers located in a single sequencing system, and the multiple carriers may share a common optical system. In the fluidics position 2503, dispenser 2070 may dispense fluids such as library solutions, samples, reagents, washing buffers, etc., onto the open landing area(s) of flow cell device disposed in the carrier 2051. The dispenser 2070 may include various embodiments of dispensing tips, e.g., pipette tips and removable cartridge for holding different reagents therein and in fluidic communication with the dispensing tips.

In some embodiments, the motion of the movable arm (e.g., at the grabber or distal tip of the movable arm) can be linear, so that the carrier 2051 moved by the moving mechanism 2040 also moves linearly. For example, the movable arm may move the carrier 2051 and the sample along x or y axis within the x-y plane. In some embodiments, the motion of the movable arm can be nonlinear, e.g., rotational in 3D, and the carrier 2051 carried by the grabber of the movable arm also moves nonlinearly correspondingly.

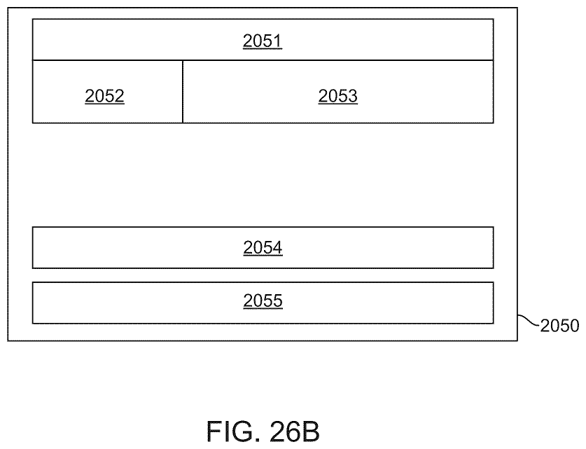

The samples disclosed herein can include various samples that are to be sequenced on the sequencing system. The sample herein may be 2D or 3D sample, including in situ samples such as cells and/or tissues. Fluids may be extracted from the flow cell device, e.g., using the extraction pump which may be connected to the carrier 2051 and the flow cell via a connection, e.g., 1006 in FIGS. 24A and 25. The connection 1006 may be a quick-connect connection with gasket(s) or other sealing components to prevent leaking and enable sealed fluidic communication. In some embodiments, the extraction pump is coupled to the nest bank. In some embodiments, the extraction pump is coupled to the flow cell.

In the imaging position 2502, the optical system may perform imaging, and there can be optional fluidic communication with the flow cell device if needed.

The flow cell device carrier may be moved between the docking positions using the moving mechanism 2040. For example, the moving mechanism 2040 may include a movable arm, a belt conveyor, roller conveyer, rail system, or pick and place robot.

In some embodiments, the sequencing system may include multiple flow cell carriers, each with their own fluidics lines 1001 connected to one or more pumps. The moving mechanism 2040 may move and manage motions of the multiple carriers 2051 using an algorithm that prevents the multiple fluidic lines 1001 from tangling or otherwise disturbing each other.

In some embodiments, the x-y stage 2010 is configured to be actuated to move to a 3D position with a predetermined spatial precision, e.g., a second spatial precision, which is different from the spatial precision of the moving mechanism 2040. The second spatial precision can be higher than the first spatial precision of the moving mechanism 2040. In some embodiments, the second spatial precision can be 2×, 4×, 5×, 6×, 8×, 10×, 15×, or more precise than the first spatial precision. For example, the first spatial precision may be 1 mm, and the second spatial precision may be 0.05 mm. Having different spatial precision may advantageously enable coarse movement of the moving mechanism 2040 to translate the sample to the stage and finer adjustment of the spatial position by the x-y stage 2010 relative to the objective lens for focusing and imaging.

In some embodiments, the x-y stage 2010 can include one or more fasteners configured to removably secure the flow cell device thereto. Various fasteners can be used herein. As a non-limiting example, the fastener(s) may comprise one or more clamps that uses mechanical (e.g., snap fit, friction fit), magnetic, or electromagnetic force(s).

In some embodiments, each carrier 2051 comprises a coupled position in which the carrier 2051 is removably attached and secured to the x-y stage 2010 by the fastener(s). In the coupled position, the carrier 2051 is in sealed fluidic communication with the x-y stage 2010. In some embodiments, each carrier 2051 comprises a decoupled position in which the carrier 2051 is removable from and not secured to the x-y stage 2010. In the decoupled position, the carrier 2051 is not in fluidic communication with the x-y stage 2010. In addition, the carrier 2051 can include one or more valves or other fluidic stoppers that prevent the fluids within the carrier 2051 from leaking out of the carrier 2051.

In some embodiments, the x-y stage 2010 does not include pumps or fluidic pathways that may connect and enable fluidic communication to the flow cell device coupled thereon. Not including pumps or fluidic pathways on the x-y stage 2010 may simplify the sequencing system and be more cost efficient. Further, separating the fluidics from the x-y stage 2010 and optics may advantageously eliminate or minimize contamination or leaking during imaging.

In some embodiments, the x-y stage 2010 can include one or more pumps configured to deliver or extract fluids from the flow cell device when the corresponding carrier 2051 is coupled to the x-y stage 2010. In some embodiments, the x-y stage 2010 can include one or more valves or stoppers that may eliminate or reduce possible leaking from the flow cell device while it is coupled on the x-y stage 2010.

In some embodiments, the x-y stage 2010 may be configured to control a temperature of the x-y stage 2010, the carrier 2051, and/or the flow cell device. In some embodiments, the x-y stage 2010 can include a heating device, a cooling device, or both. In some embodiments, the x-y stage 2010 may be configured to maintain the temperature of the carrier 2051 and/or flow cell device at a predetermined temperature and/or within a predetermined temperature range. In some embodiments, the x-y stage 2010 may include one or more temperature sensors. The one or more temperature sensors may monitor a temperature of the x-y stage 2010, the carrier 2051, and/or the flow cell device and send a signal to a processor coupled to the x-y stage 2010 to adjust the temperature (e.g., of the heating device and/or cooling device) to achieve a predetermined temperature. Various thermal devices and sensors can be used herein.

In some embodiments, the x-y stage 2010 is coupled to a mechanical decoupler that is configured to isolate the x-y stage 2010 from vibration or other mechanical disturbance external to the x-y stage 2010. Suitable mechanical decouplers are known in the art, and include, inter alia, air tables.

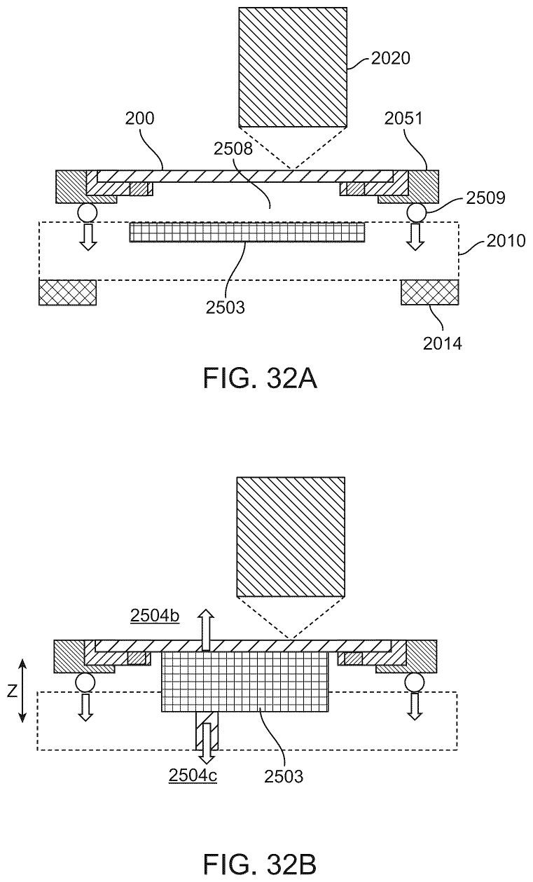

FIG. 32A shows an exemplary embodiment of the x-y stage 2010 that is coupled to a mechanical decoupler or mechanical isolator 2014. The mechanical isolator 2014 may prevent or otherwise minimize external mechanical disturbance, e.g., vibrations, from reaching the flow cell device 200 and/or the optical system 2020 so that disturbance external to the flow cell device 200 may not interfere with imaging and/or cause undesired motion of the sample(s) immobilized on the flow cell device 200. Various mechanical isolators 2014 can be used for isolating external mechanical disturbances.

In some embodiments, the x-y stage 2010 may include one or more mechanisms for capturing stray light from the optical system 2020. In some embodiments, the x-y stage 2010 comprises one or more beam dump devices (e.g., 2503 in FIGS. 32A-32B) that is configured to capture energy from the light source of the optical system, preventing the excitation light from returning to the optical system (e.g., image sensor) and producing noise signals in flow cell images. In some embodiments, the beam dump device(s) captures excitation light that travels from the optical system through the flow cell device 200 and then to the beam dump device(s). In some embodiments, the beam dump device(s) captures emission light that is emitted from the sample and travels to the beam dump device(s). In some embodiments, the beam dump device can include a laser beam dump device. In some embodiments, the beam dump device can comprise various components that are configured to absorb photon energy, such as graphite, tungsten, concrete, marble, etc.

In some embodiments, the optical system 2020, or some structural element of the optical system, e.g., the object lens, may be positioned above the flow cell device 200, as shown in FIGS. 32A-32B. In some embodiments, the optical system 2020 may be positioned in various different locations relative to the flow cell device 200, and should not be limited to the embodiment shown in FIG. 32A-32B. For example, the optical system 2020, or some structural element of the optical system, e.g., the object lens, may be positioned underneath the flow cell device 200, while the x-y stage 2010 may be located above the flow cell device 200 or to the side of the flow cell device 200.

In some embodiments, the x-y stage 2010 may include one or more mounting elements 2509 configured to secure the carrier 2051 to the x-y stage 2010. FIG. 32A shows a schematic diagram of an example embodiment of the x-y stage 2010 and the carrier 2051. In some embodiments, the mounting element(s) 2509 can include precision kinematic mounts. In some embodiments, the mounting element(s) 2509 can be configured to accurately maintain the position of the carrier 2051 relative to the x-y stage 2010. In some embodiments, the mounting element(s) 2509 may be secured to the x-y stage 2010 via various securing elements such as magnetic latches.

In some embodiments, the mounting element(s) 2509 may include a Maxwell coupling or a Kelvin coupling. In some embodiments, the mounting element(s) 2509 may include a precision kinematic mounting element. In some embodiments, the mounting element(s) 2509 have a total of less than 10, 9, 8, 7, 6, 5 contact points with the x-y stage 2010. In some embodiments, the mounting element(s) have a total of less than 6, 5, 4, or 3 contact points with the x-y stage 2010. In some embodiments, the mounting element(s) 2509 include various geometrical shapes that satisfy the principle of exact constraint design. For example, in some embodiments, the number of points of constraint of the mounting element(s) 2509 is equal to the number of degrees of freedom of the flow cell device carrier 2051 to be constrained. As an example, the mounting element(s) 2509 may include three elements, each having a spherical surface that rest respectively on a concave tetrahedron, a V-groove pointing towards the tetrahedron and a flat surface of the x-y stage 2010. The tetrahedron may provide three contact points, while the V-groove provides two and the flat provides one, for a total required six contact points with the x-y stage 2010. As another example, the x-y stage 2010 may comprise three V-shaped grooves, while the mounting element(s) 2509 include three elements each having a curved surface that is configured to be positioned on a corresponding grooves. Each of the three v-grooves provides two contact points with the corresponding mounting element 2509 for a total of six contact points. In some embodiments, the mounting element(s) 2509 are configured to control precision of the location of the carrier 2051 to be within a range from 0.1 nm to 0.1 mm, inclusive of all ranges and subranges therebetween. In some embodiments, the mounting element(s) 2509 are configured to control precision of the location of the carrier to be within a range from 1 nm to 1 μm, inclusive of all ranges and subranges therebetween. In some embodiments, the mounting element(s) 2509 are configured to control precision of the location of the carrier 2051 to be within a range from 0.1 μm to 0.1 mm, inclusive of all ranges and subranges therebetween. In some embodiments, the mounting element(s) 2509 are configured to control precision of the location of the carrier 2051 to be within a range from 1 μm to 0.1 mm, inclusive of all ranges and subranges therebetween.

In some embodiments, there may be a gap zone 2508 between the flow cell device 200 and the x-y stage 2010 when the flow cell device 200 and its carrier 2051 are in the imaging position. The gap zone 2508 lacks any heater/cooler connection or fluidic connections to the flow cell device 200 or its carrier 2051 The gap zone 2508 is maintained to reduce the mechanical and thermal stresses that may be exerted on the flow cell device 200 from the heater/cooler, or fluidic pathways, so that flow cell images can be acquired with less thermal or mechanical disturbance when the flow cell device 200 is in the imaging position. Therefore, the gap zone 2508 may improve image quality of the flow cell images by reducing mechanical disturbances (e.g., vibration, shaking, warping of the flow cell device 200, etc.) and by reducing thermal disturbances (e.g., uneven temperature of the flow cell device 200, excess or stray infrared energy, etc.). The height of the air gap (e.g., along z axis) can be predetermined. The height of the air gap may be adjustable by adjusting the relative position of the flow cell device 200 to the x-y stage 2010. The air gap can have a height from 0.01 mm to 1 cm, inclusive of all ranges and subranges therebetween. The air gap can have a height from 0.1 mm to 5 cm, inclusive of all ranges and subranges therebetween. The air gap can have a height from 1 mm to 50 cm, inclusive of all ranges and subranges therebetween.

FIG. 32B shows another embodiment of the x-y stage 2010, also referred to as the imaging dock 2010. In this embodiment, the x-y stage 2010 includes a beam dump 2503. This beam dump 2503 may make direct contact with the flow cell device 200, the carrier 2051, or both. In some embodiments, the beam dump 2053 may contact the flow cell device 200 and/or carrier 2051 with a minimal contact force, or any force within a predetermined force range. Without wishing to be bound by theory, it is thought that minimal contact force with beam dump reduces mechanical stress and lowers the potential for thermal or vibrational transfer. The contact with predetermined force may be maintained by balancing a latching force 2504b to be just slightly larger than a damping force 2504c, both the latching force 2504b and damping force 2504c can be applied to the beam dump 2503 via various mechanisms. For example, the latching force 2504b may be applied through one or more magnetic latch(es) located in the flow cell device 200, the carrier 2051, and/or the x-y stage 2010. The magnetic latches may pull the carrier 2051 thus the flow cell device 200 toward the x-y stage 2010 and/or pull the x-y stage 2010 toward the carrier 2051.

In some embodiments, the beam dump 2503 may generate a damping force 2504c that pulls the beam dump away from the flow cell device 200 and the carrier 2051. In some embodiments, the damping force 2504c may be generated by using a biasing mechanism (e.g., springs deformable members, pliable material, etc.). In some embodiments, the damping force 2504c can counteract the latching force 2504b. The net effect of the damping force 2504c and latching force 2504b may be used to minimize the amount of force imparted upon the flow cell 200. The total force by combining the latching 2504b and damping force 2504c can be tuned, for example, by selecting various magnets and spring combinations.

In some embodiments, the latching force 2504b and the damping force 2504c may be within a predetermined range. In some embodiments, the net force combining the latching 2504b and damping force 2504c may be within a predetermined range. In some embodiments, the net force may be in a range from −0.001 Newtons (N) to 0.001 N (e.g., positive force is upward toward the flow cell device along z), inclusive of all ranges and subranges therebetween. In some embodiments, the net force may be in a range from −0.01 N to 0.01 N (e.g., positive force is upward toward the flow cell device along z), inclusive of all ranges and subranges therebetween. In some embodiments, the net force may be in a range from −0.05 N to 0.05 N (e.g., positive force is upward toward the flow cell device along z), inclusive of all ranges and subranges therebetween. In some embodiments, the net force may be in a range from −0.5 N to 0.5 N (e.g., positive force is upward toward the flow cell device along z), inclusive of all ranges and subranges therebetween. In some embodiments, the net force may be in a range from −10 N to 10 N (e.g., positive force is upward toward the flow cell device along z), inclusive of all ranges and subranges therebetween.

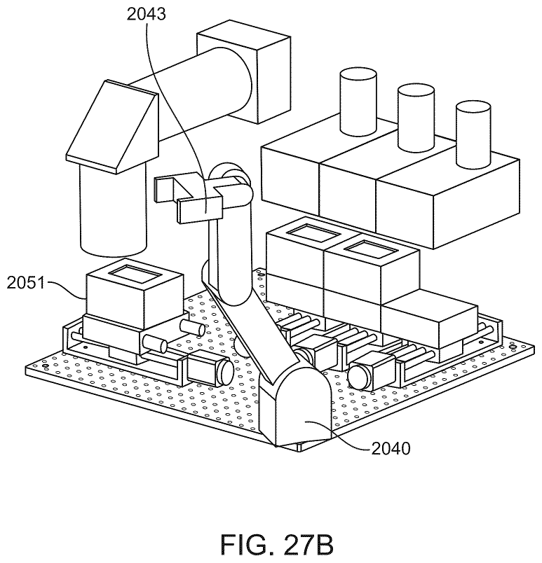

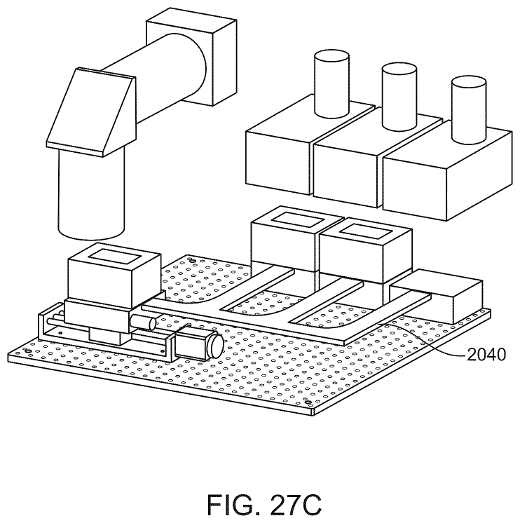

FIG. 26A shows a schematic diagram of an example of the x-y stage 2010. The x-y stage 2010 may secure the carrier 2051 using a coupling mechanism 2012, e.g., clamps, fasteners, bolts, anchors, rivets, etc. The x-y stage 2010 may include a temperature controller (e.g., a heating and/or cooling device) 2013 that is optimized to maintain a predetermined temperature of the sample for incubation during library preparation, for imaging, etc. The x-y stage 2010 may include a position adjustment device 2014, e.g., a tip-tilt device, which positions the carrier 2051 relative to a 3D position with a predetermined precision, e.g., the first precision, for imaging. In some embodiments, the x-y stage 2010 is motorized or otherwise actuated by an actuator 2015 for translation and or rotation to position the carrier 2051 in 3D for imaging. In some embodiments, the x-y stage 2010 is actuated to move via the actuator 2015 to a location with a precision that is relatively lower but sufficient to get the sample to the vicinity of the predetermined position (e.g., the imaging position). The position adjustment device 2014 then fine tunes the position of the sample with a higher precision to the predetermined location with a higher precision. The combination of the movement of the x-y stage 2010 (and the sample) relative to the housing or any other reference point and the movement sample relative to the x-y stage 2010 allow the sample to be positioned accurately and reliably at the predetermined 3D location for imaging.

In some embodiments, the x-y stage 2010, the moving mechanism (e.g., moving mechanism 2040 not shown), or both are actuated to move with a precision that is relatively lower but may get the sample to the vicinity of the predetermined location, e.g., within several millimeters to the predetermined location. The position adjustment device 2014 located on the x-y stage 2010 then fine tunes the position of the sample with a higher precision to the predetermined location with a higher precision. In some embodiments, the combination of the movement of the stage (and the sample) relative to the housing or any other reference point and the movement sample relative to the stage allow the sample to be positioned accurately and reliably at the predetermined 3D location for imaging.

As a nonlimiting example, while the carrier 2051 is coupled with the nest bank 2050 for fluidic administration to the corresponding flow cell, the moving mechanism 2040 may be prevented from moving the carrier 2051 away from the nest bank 2050, the x-y stage 2010 may move to a position between the optical system 2020 and the nest bank 2050 to wait for the sample and save some traveling distance and time for the moving mechanism carrying the sample. After fluidic administration is completed, the moving mechanism 2040 may be allowed to move the carrier 2051 to the x-y stage 2010, and then the x-y stage 2010 can move back to the optical system 2020 to a position for imaging the sample. In this particular embodiment, the position adjustment device 2014 may then fine tune the position of the sample relative to the objective lens for imaging while the x-y stage 2010 and the moving mechanism 2040 may move the sample in lower spatial precision, optionally with possible higher speed if needed to save time. Such arrangement of movement with different spatial precision may advantageously reduce system complexity, save manufacturing cost, improve robustness of the sequencing system and reduce time of the sequencing run for users.

In some embodiments, at least some parts within the optical system 2020, the x-y stage 2010, or both are mounted on a vibration isolator(s) that mechanically decouples the rest of the instrument for the purposes of improving image quality. Such isolation may advantageously allow imaging with minimal motion disturbance and may also facilitate fluidics dispensing and chemistry processing without external motion disturbance. In some embodiments, the flow cell device is coupled to a carrier, the movable arm is configured to move the carrier and the flow cell device together. The carrier 2051 may remain fixedly coupled to the flow cell device when the flow cell device is on the x-y stage 2010. In some embodiments, the optical system 2020 may move linearly or nonlinearly in 3D. For example, the optical system 2020, or at least part of the optical system may move along x, y, or any other linear axis in 3D. As another example, at least part of the optical system may rotate in 3D about z axis or other axis in 3D. For example, the x-y stage 2010 may move linearly along the x axis and the optical system (e.g., at least the objective lens) can move linearly along y axis, and the combination of movements of the optical system and the x-y stage 2010 allow the sample to be positioned at a predetermined position relative to the objective lens for imaging. As another example, one of the x-y stage 2010 or the optical system 2020 may move nonlinearly, e.g., rotate about a predetermined origin, and the other one of the x-y stage 2010 or the optical system 2020 may move linearly and the combination of movements of the optical system and the x-y stage allow the sample to be positioned at a predetermined position relative to the objective lens for imaging.

In some embodiments, the nest bank 2050 of the sequencing system can hold flow cell devices thereon and preparing flow cell devices and samples thereon for imaging during a sequencing run.

The nest bank can be in fluidic communication with various reagents and buffers, e.g., washing buffers and/or library loading buffers. The fluidic communication between nest and reagent or solution containers may be via closed fluidic pathways or open fluidic communication in which fluids can be dispensed openly.

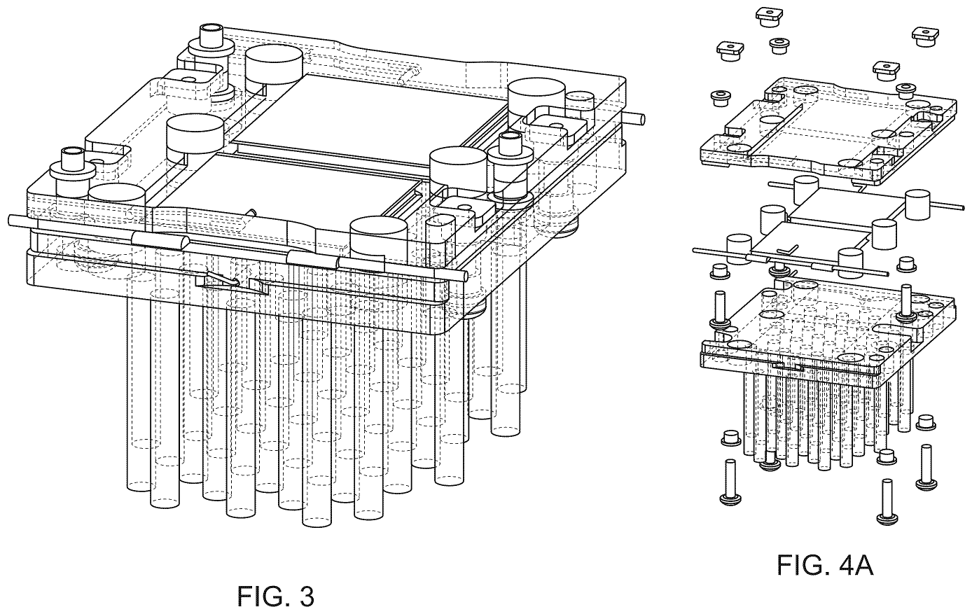

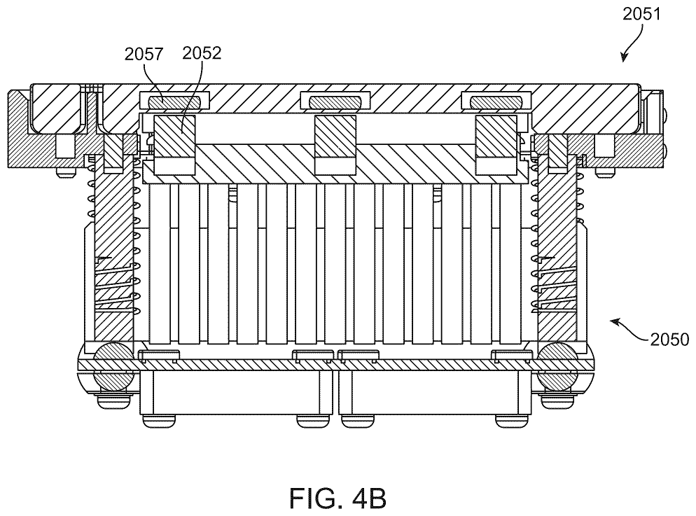

In some embodiments, each nest module of the nest bank 2050 comprises a thermal and fluidics interface to the flow cell device. The nest modules can be distributed linearly in an array as shown in FIG. 27A or of various other spatial distributions. Thermal incubation can occur within a chamber corresponding to an individual flow cell device carrier. One or more heat sinks can be positioned below each nest module as shown in FIGS. 3 and 4A. Fluidics interface can include dispensing tips that dispense into the open wells of the flow cell device or plug-ins that couples to respective microfluidic channels of the flow cell device.

Each of the flow cell devices may comprise an open landing area configured for receiving fluids openly from the nest bank. The flow cell device comprises a plurality of microfluidic channels, and the nest bank is configured to allow fluidic communication to each of the plurality of microfluidic channels. The fluidic communication from the nest bank to each channel may be independent so that cross contamination can be avoided. For example, different pipette tips can be used to dispense different reagents to different channels via corresponding openings of the nest bank. In some embodiments, the fluidic communication from the nest bank to multiple channels may be simultaneous to reduce fluidic communication time in a sequence run. In some embodiments, the fluidic communication from the nest bank to multiple channels may be sequential to simplify the communication process and reduce complexity and cost of the nest bank.

FIGS. 24A-24B show exemplary embodiments of the carrier 2051 described herein. In some embodiments, e.g., in FIG. 24A, the carrier 2051 can include an opening 1004 at a surface of the carrier 2051 configured to receive a flow cell device (not shown) therein. The carrier 2051 can further include one or more fluidic pathways 1001 in sealed fluidic communication with the flow cell device when the flow cell device is removably attached to the carrier 2051. The carrier 2051 may include a pump 1003 configured to pull or push fluids between the flow cell device and the carrier 2051. The flow cell device carrier can include a port opening 1006 with a connector that is configured to enable sealed fluidic communication between the carrier 2051 and the corresponding nest module when the connector is in a connected position. The carrier 2051 can include a valve 1005 positioned between a fluidic pathway connecting to the flow cell device and a port opening of the carrier 1006, wherein the valve that is in an open position when the flow cell device is in the coupled position to the carrier (e.g., not during imaging); and in a closed position when the flow cell device is in the decoupled position (e.g., during imaging). The carrier 2051 can include electric wiring 1007 with an electric connector 1008 configured to enable electric communication between the carrier 2051 and a power supply. The carrier 2051 may include an on-board battery and/or sensor 1009 in electric communication with one or more of the elements, e.g., the pump 1003, the port 1006, and the fastener (e.g., fastener 2012). One or more of battery, sensor, pump, and fastener(s) may be connected with the electric connector 1008 via the electric wiring to an external power supply.

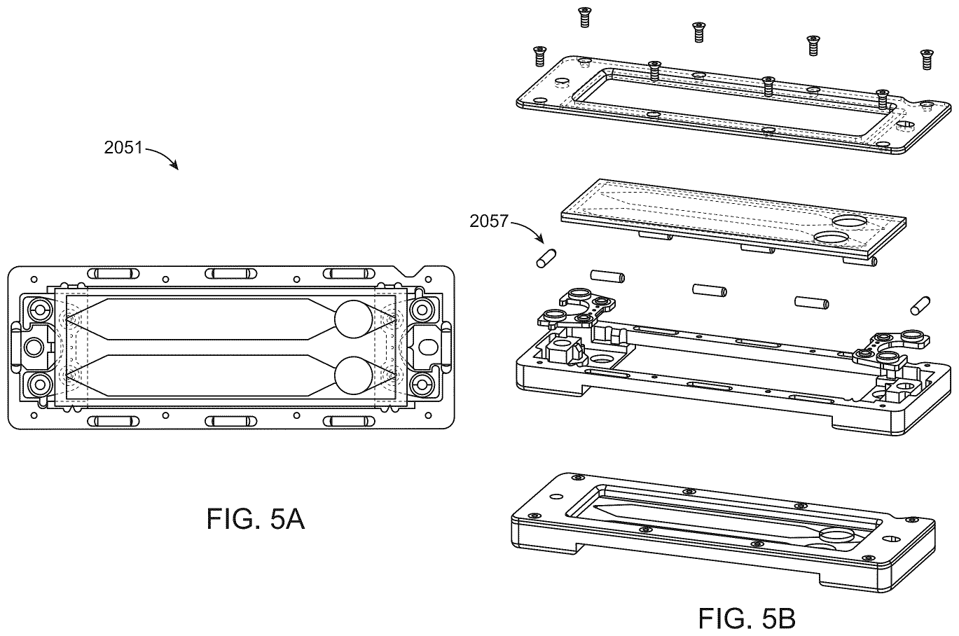

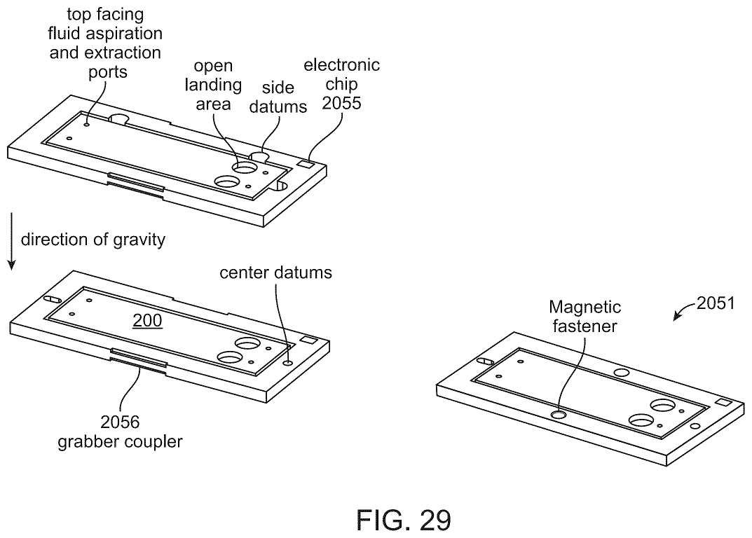

FIGS. 5A-5B and FIG. 29 show exemplary embodiments of the carrier 2051. In the embodiment shown in FIG. 29, the carrier 2051 may not include a valve or similar structures that functions similarly. In this particular embodiment, open landing area for aspiration and/or outlet for fluid extraction may be oriented upwards, facing away from the direction of gravity. Further, the fluidic port (e.g., similar to port 1006) may also face upward. Such structural arrangement may help prevent fluid loss without needing valves on the carrier 2051. The upward facing port(s) may function equivalent as the fluidic ports 1006 fluid extraction ports. In this embodiment, a grabber coupler 2056 may be located on the carrier to aid the grabber (e.g., of the movable arm) in securely grabbing the carrier 2051 while moving from the fluidics station to the imaging station. The grabber coupler 2056 may be mechanical, electro-mechanical, or magnetic.

In some embodiments, the one or more fasteners may include one or more locating datums. FIG. 29 shows one or more side locating datums and/or center locating datums may be used to securely locate the flow cell relative to the x-y stage (not shown) or the nest bank (not shown).

In some embodiments, the carrier 2051 may include an electronic chip 2055 embedded into the carrier to identify the flow cell, e.g., the serial number or any other unique ID, and any relevant sequencing information, such as sequencing cycle, and identification for the assay. The electronic chip 2055 may be in electronic communication with the processor(s) of the sequencing system. The electronic chip 2055 may be in electrical communication with a power source, e.g., a battery on the carrier 2051 or a power outlet external to the carrier.

In some embodiments, the carrier 2051 may be equipped with one or more sensors to provide feedback of the alignment of the carrier 2051 relative to the x-y stage and/or the optical system. Various sensors can be used, such as cameras, audio sensors, light sensors, thermal sensors, radio frequency sensors, pressure sensors, and/or force sensors. The feedback of one or more sensors may be sent to the processor of the sequencing system, and the feedback can be used to fine tune the positioning of the carrier 2051 and the flow cell device therein relative to the x-y stage and/or the optical system so that the sample(s) is aligned with the objective lens for imaging. For example, an audio sensor may be used to detect sound waves bounced back from the carrier to see how far off the carrier is from the aligned imaging position. As another example, camera(s) may be used to detect whether the carrier is positioned at the aligned imaging position or not.

In some embodiments, the carrier 2051 may include a hardware processor that is different from and external to the processor of the sequencing system. The hardware processor of the carrier 2051 may communicate with the one or more sensors so that the carrier 2051 does not need to communicate with the processor of the sequencing system to process the feedback from the one or more sensors, and generate instructions the actuators(s) (e.g., the tip-tilt device, the motor) to move the carrier to desired 3D locations, e.g., relative to the nest bank. The movement can be with the predetermined precision. In some embodiments, the carrier 2051 may include a hardware processor that is different from and external to the processor of the sequencing system to enable autonomous or semi-autonomous movement of the carrier 2051 based on the feedback(s) of the one or more sensors. A separate processor for the carrier 2051 may also help to distribute computing power and increase operation speed of the sequencing system.

In some embodiments, the carrier 2051 may not include a hardware processor external to the processor of the sequencing system. The one or more sensors may communicate with the processor of the sequencing system to process the feedback from the one or more sensors, and generate instructions the actuators(s) (e.g., the tip-tilt device, the motor) to move the flow cell device carrier to desired 3D locations.

In some embodiments, e.g., in FIG. 24B, a pump 1003 is located external to the carrier 2051 and may or may not move with the carrier 2051. Having external pump 1003 may advantageously allow the carrier 2051 to be simpler, more compact in size, and with less weight so that it is easier to be moved by the moving mechanism. In some embodiments, other elements in FIG. 24A, such the battery may be positioned external to the carrier 2051. In some embodiments, the fluidic pathway may include tubing, e.g., flexible or semi-flexible, and allow permanent connection to the pump 1003.

In some embodiments, the nest bank 2050 is configured to enable fluidic and thermal communication with the one or more flow cell devices. In some embodiments, the nest bank is configured to enable fluidic and thermal communication with various numbers of flow cell devices. For example, at least 1, 2, 3 4, 5, 6, 7, 8, 9 or 10 flow cell devices. For example, FIG. 2 shows a nest bank that is configured to hold 3 flow cell devices.

The nest bank 2050 is configured to hold each of the flow cell devices in an unlocked position in which the flow cell device is removable from the nest bank. The nest bank is configured to hold a flow cell device in a locked position in which the flow cell device is spatially registered to the nest bank, fixedly coupled to the nest bank, and sealed fluidic communication and thermal communication between the nest bank and the flow cell device are enabled. The nest bank 2050 is configured to transition between the unlocked position and the locked position as the flow cell device is disposed in a portion of the nest bank (e.g., by the moving mechanism). In some embodiments, the nest bank 2050 may automatically transition from the unlocked position to the locked position upon the flow cell device being disposed on or near a portion of the nest bank. In some embodiments, sequencing (e.g., flow of reagents) may be prevented until the flow cell device is transitioned to the locked position in the nest bank 2050, as described below.

The carrier 2051 is configured to be spatially registered to the nest bank in the locked position thereby spatially registering the corresponding flow cell device therewithin to the nest bank 2050. FIGS. 5A-5B show a top view and an exploded view of a flow cell device coupled to a corresponding carrier 2051. In this embodiments, pins embedded in the frame of the carrier 2051 are configured to clamp the pieces of the carrier 2051 and/or flow cell device together. Ribs in the frame of the carrier 2051 can push flow cell device in a registered position relative to the carrier 2051, and therefore the registered position relative to the nest bank 2050.

The nest bank 2050 may include one or more fasteners. The one or more fasteners may use various mechanisms to fasten the carrier and corresponding flow cell device to the nest bank 2050. The one or more fasteners may use magnetic force. FIG. 3 and FIGS. 4A-4B show an exemplary embodiment of the nest bank in which the one or more fasteners 2052 (also referred to in FIG. 26B) may include multiple magnets. Each magnet can be a rare earth magnet, an electromagnetic coil, or both, for example. In some embodiments, the one or more fasteners can be switched on or off. For example, the one or more fasteners are controlled by the one or more processors to switch between an on-stage and an off-stage. In some embodiments, the one or more fasteners lack mechanical fasteners that can be actuated by a physical actuator like a motor, a clamp, a spring, etc. The one or more fasteners 2052 may be pushed to ensure it is in the locked position by the pins 2057 as shown in FIG. 4B. The one or more fasteners, alone or in combination with the push pins, ensure that the flow cell device and its corresponding carrier 2051 is in the locked position relative to the nest bank 2050 so that it is spatially registered to the nest bank 2050, and sealed fluidic communication and thermal communication (e.g., in physical contact with the heat sink) are enabled in such a locked position.

In some embodiments, the nest bank 2050 may include one or more fasteners, each configured to removably fasten and secure a corresponding carrier to the nest bank. Various fasteners may be used herein. For example, each fastener may include one or more clamps. The one or more clamps may be actuated by different forces, such as magnetic or electromagnetic forces.

In some embodiments, the nest bank 2050 may include one or more pumps configured to enable fluidic communication with the flow cell device when the corresponding the carrier 2051 is coupled to the nest bank 2050. Each flow cell device carrier may include a coupled position in which the carrier 2051 is removably attached and secured to the nest bank 2050 via the fasteners, and in sealed fluidic communication with the nest bank. Each carrier 2051 may comprise a decoupled position in which the flow cell device carrier is removable from the nest bank 2050. In the decoupled position, the flow cell device carrier is not in fluidic communication with the nest bank 2050, and the fluids within the carrier 2051 are sealed from leaking out. Sealing may be enabled by one or more valves, e.g., 1005 in FIG. 24A.

The nest bank 2050 may comprise a 3D movement device that is configured to position the carrier 2051 relative to the rest of the nest bank 2050 with a third spatial precision, while the carrier 2051 remain coupled to the nest bank 2050. The third spatial position can be greater than the first spatial precision of the movable arm by 2×, 3×, 4×, 5×, 6×, 7×, 8×, 9×, 10×, 15×, or more.

In some embodiments, the nest bank 2050 may comprise a 3D movement device that is configured to position the carrier 2051 relative to the dispensing tips while remain coupled to the nest bank with a fourth spatial precision. The movement may advantageously fine tune position of the open landing areas(s) of the flow cell device relative to the dispensing tip(s) to ensure secure and accurate fluidic administration to the flow cell device. The fourth spatial position can be greater than the first spatial precision of the movable arm by 2×, 3×, 4×, 5×, 6×, 7×, 8×, 9×, 10×, 15×, or more. In some embodiments, 3D movement device can actuate the carrier 2051 to translate in 3D or rotate about an axis in 3D. For example, the 3D movement device may be a piezo stage or actuator that can move along z, tip or tilt within a predetermined angular range.

In some embodiments, the nest bank 2050 may be equipped with one or more sensors to provide feedback of the alignment of the carrier 2051 relative to the nest bank 2050. Various sensors can be used, such as cameras, audio sensors, light sensors, thermal sensors, radio frequency sensors, pressure sensors, and/or force sensors. The feedback of one or more sensors may be send to the processor of the sequencing system, and the feedback can be used to fine tune the positioning of the flow cell device carrier and the flow cell device therein relative to the nest bank 2050 so that the sample(s) is aligned to be coupled securely with the nest bank 2050. For example, an audio sensor may be used to detect sound waves bounced back from the carrier to see how far off the carrier 2051 is from the aligned position relative to the nest bank 2050. As another example, camera(s) may be used to detect whether the carrier 2051 is positioned at the aligned position relative to the nest bank 2050 (and/or dispenser 2070) or not.

It is worth noting that the sensors may be equipped with one or more structural elements of the sequencing systems, such as the x-y stage 2010, the moving mechanism, the carrier 2051, and the nest bank 2050, in order to facilitate efficient and accurate positioning of the carrier 2050 relative to the x-y stage 2010 and/or the optical system 2020 and to facilitate efficient and accurate positioning of the carrier 2051 relative to the nest bank 2050.

Alternatively, the nest bank 2050 may not include such 3D movement device that moves the carrier 2051 relative to the rest of the nest bank 2050 or the dispensing tips. Instead, the dispenser 2070 may be equipped with a 3D movement device that is configured to move the dispensing tips in 3D while the nest bank 2050 and carrier 2051 remain fixed relative to a reference point. As a result, the dispensing tips and open landing areas are aligned relative to each other for accurate and secure fluidic administration.

FIG. 26B shows a schematic illustration of the nest bank 2050 with one nest bank modules 2051. Disclosed herein, the nest bank module is equivalent to a flow cell device carrier (e.g., carrier 2051) docked in the nest bank. Although not shown in this embodiment, more than one nest bank module, or equivalently carrier 2051, can be included in the same nest bank.

The nest bank may secure each carrier 2051 independently using some clamping mechanism 2052. In some embodiments, the flow cell device may include an open landing area that receives open dispensing by a dispenser 2070 as shown in FIG. 27A. In such embodiments, the nest bank may include a pump and optional valves to sealedly mate with the flow cell carrier and extracts fluidic, e.g., waste, from the flow cell while the carrier is in the coupled position relative to the nest bank.

FIG. 31 shows a schematic illustration of the nest bank 2050 with one nest bank module 2051. In this particular embodiment, the nest bank module, or equivalently herein, the carrier 2051 is in the locked position with the nest bank 2050. As shown in FIG. 31, in some embodiments, the flow cell device 200 may include a flow cell frame 2092 that encloses at least part of the flow cell device 200 therewithin, e.g., one or more edges of the flow cell device 200, and some area of the flow cell device 200 along the x-y plane. The flow cell frame 2092 can include various materials including but not limited to metal, plastic, silicone, and rubber. In some embodiments, the flow cell frame 2092 is configured to hold one or more gaskets 2093 in position relative to the flow cell device 200. The gasket(s) 2093 may facilitate sealed fluidic communication between the flow cell device 200 and the nest bank 2050. In some embodiments, the gasket(s) 2093 may be connected with a fluidic manifold 2095 of the nest bank 2050. The fluidic manifold 2095 may be in fluidic communication with one or more fluidic lines 1001 connected to one or more pumps.

In some embodiments, the carrier 2051 may include one or more fastener(s) or docking features 2094 that may be coupled with the matching features in the nest bank 2050 for securely coupling the carrier to the nest bank 2050. Various mechanical elements can be used as the fastener or docking features such as, for example, clamps, clips, bolts, magnets, snap fit components, latches, adhesive, etc. Exemplary docketing features 2094 may include alignment detents and magnetic latches. The docking features may be configured to maintain a reliable connection (e.g., sealed and aligned) from the flow cell gasket 2093 to the fluidic manifold 2095. The docking features may be configured to maintain a reliable connection (e.g., sealed and aligned) from the flow cell device 200 to a heater and/or cooler 2053 of the nest bank 2050.

Similar as the x-y stage (not shown), the nest bank 2050 may include a heating and/or cooling device 2053 that is optimized to develop the fluids and assays for chemistry processing. In some embodiments, the nest bank may further include a temperature controller (e.g., a cooler, a heater, or both). The cooler or heater is configured to control temperature of each sample immobilized on the one or more flow cell devices. The cooler or heater may comprise various sources for heating or cooling the sample. For example, the cooler or heat may include at least one of a fan configured to blow cool or hot air, a microwave, an infrared light source, and/or an electromagnetic wave source. In some embodiments, the temperature controller of the nest bank may be structurally and/or functionally similar to the temperature controller of the x-y stage.

As shown in FIG. 26B, similar to the x-y stage, the nest bank 2050 may include a position adjustment device 2054, e.g., a tip-tilt device, which positions the carrier 2051 to a 3D position with a predetermined precision, e.g., the first precision, for imaging. The position adjustment device 2054 herein is configured to travel along z axis, tip, and/or tilt with a predetermine angle range for fast and accurate multi-axis positioning. The tip or tilt may be about an axis in 3D, e.g., about x, y, or other axis within the x-y plane. The tip or tile angle may be in various ranges. For example, the tip or tilt angle may be greater than 0 but smaller than 0.001 mrad, 0.01 mrad, 0.1 mrad, 1 mrad, 10 mrad, 100 mrad, or 200 mrad.

In some embodiments, for example those embodiments with in situ samples, the fluidic station or at least a portion of the fluidic station may be external to the housing of the sequencing system to facilitate sample preparation. In some embodiments, the dispenser (e.g., dispenser 2070) and the nest module may be external to the housing of the sequencing system (at least partially) so that sample preparation can be monitored during its progress.

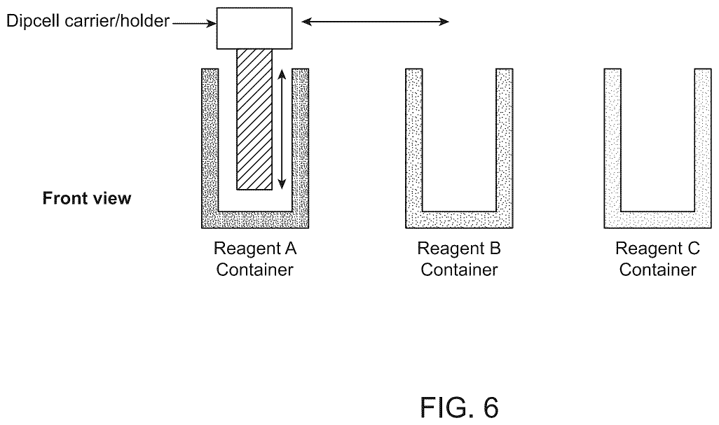

In some embodiments, the nest bank 2050 comprises one or more reagent containers. In some embodiments, the one or more reagent containers may be disposable. In some embodiments, the moving mechanism (e.g., the movable arm) is configured to dip a flow cell device into at least some of the one or more reagent containers. For example, as shown in FIG. 6, the nest bank can include three different reagent containers for each flow cell device, and the moving mechanism may carry the flow cell device and dip it into each container with predetermined time duration and temperature. Some of the reagent containers may hold washing buffer(s) therewithin to reduce cross contamination between buffers. For example, the first flow cell device may be dipped into reagent A container, and then moved to the x-y stage for imaging, and then dipped into reagent B container for washing, and then reagent C container for flowing a second type of reagent into the microfluidic channels.

In some embodiments, the sequencing system may not include a moving mechanism comprising a movable arm that moves the flow cell device carrier between the nest bank (e.g., nest bank 2050) and the optical system (e.g., optical system 2020). In such alternative embodiments, the optical system may move relative to the nest bank in order to position a flow cell at a predetermined position relative to the objective lens for imaging. For example, the optical system may move linearly along y axis and the nest bank or the flow cell carrier, decoupled from the nest bank may move linearly along x axis and their combined movement allow the sample to be positioned for imaging under the objective lens. As another example, the optical system may be fixed relative to the housing or another reference point of the sequencing system, and the x-y stage and/or the flow cell device carrier may move linearly in the x-y plane to allow the sample to be positioned for imaging under the objective lens. As yet another example, the optical system may be fixed relative to the housing or another reference point of the sequencing system, and the x-y stage and/or the flow cell device carrier may move nonlinearly in the x-y plane, e.g., rotate z axis, and position the flow cell device carrier relative to the fixed optical system for imaging.

It is worth noting that the movable arm, the x-y stage, the nest bank, and the flow cell device carriers herein may be independently actuated into linear or nonlinear movement(s) so that the combination of their movements can position the sample on the flow cell relative to the objective lens in 3D for imaging. In some embodiments, the dispenser that holds a volume of reagent(s) and dispense into the flow cell device moves together with the nest bank so that it remains fixed relative to the nest bank to ensure proper fluidic administration into the flow cell when the flow cell carrier is coupled to the nest bank.

Disclosed herein are sequencing methods using the sequencing system herein. The sequencing methods may comprise one or more operations disclosed herein. The methods disclosed herein can include some or all of the operations disclosed herein. The operations may be performed in, but are not limited to, the order that is described herein.

The methods can be performed by one or more hardware processors disclosed herein. In some embodiments, the processor can include one or more of: a processing unit, an integrated circuit, or their combinations. For example, the processing unit can include a central processing unit (CPU) and/or a graphic processing unit (GPU). The integrated circuit can include a chip such as a field-programmable gate array (FPGA). In some embodiments, the processor can include the computer system 126.

In some embodiments, some or all operations in method can be performed by the FPGA(s) (e.g., FPGA(s) 120). In embodiments when some operations are performed by FPGA(s), the data after an operation performed by the FPGA(s) can be communicated by the FPGA(s)s to the CPU(s) (e.g., CPU(s) of the computer system 126) so that CPU(s) can perform subsequent operation(s) in method 500 using such data. Similarly, data can also be communicated from the CPU(s) to the FPGA(s) for processing by the FPGA(s). In some embodiments, all the operations in method 500 can be performed by CPU(s). Alternatively, the operations performed by CPU(s) can be performed by other processors such as the dedicated processors, or GPU(s). In some embodiments, all the operations in method 500 can be performed by FPGA(s).

In some embodiments, the methods herein may include operations of: (a) moving a first flow cell device from the nest bank to the x-y stage, wherein the first flow cell device comprises a first sample immobilized thereon; (b) moving the x-y stage and the first sample thereon relative to an objective lens of an optical system of a sequencing system; (c) imaging the first sample immobilized on the first flow cell device on the x-y stage using the optical system; (d) moving the first flow device from the x-y stage to the nest bank; and (e) allowing (e.g., simultaneously) fluidic and thermal communication between the nest bank and a second flow cell device during one or more of: (a)-(d).

In some embodiments, the operations (a) to (d) occur during a same flow cycle of a sequence run. In some embodiments, operation (e) occurs within the same flow cycle as operations (a) to (d). Operations can be repeated in each individual cycle of a sequence run. For example, operations can be repeated at least once within each cycle. As another example, some of the operations can be repeated more than once within a single cycle. For example, moving the flow cell device to the x-y stage may occur after each reagent administration to the flow cell device within a same cycle.

In some embodiments, some of the operations may occur immediately after its preceding operation is completed to avoid waste in time in performing a sequence run. For example, immediately after a first reagent is administered to a first sample, even if a second sample is being imaged, the movable arm can move the first sample to a location close to the x-y stage and the objective lens so that it can be quickly moved to the imaging position after the second sample has been imaged.

Although one movable arm is depicted in the exemplary embodiment in FIG. 2, more than one movable arm can be included, and each arm may move one or more corresponding flow cell devices to improve system efficiency and throughput and reduce idling time of the imaging system.

In some embodiments, the methods further includes (f) moving a second flow cell device from the nest bank to the x-y stage, wherein the second flow cell device comprises a second sample immobilized thereon; (g) moving the x-y stage and the second sample thereon relative to an objective lens of an optical system of a sequencing system; (h) imaging the second sample immobilized on the second flow cell device on the x-y stage using the optical system; (i) moving the first flow device from the x-y stage to the nest bank; and (j) allowing (e.g., simultaneously) fluidic and thermal communication between the nest bank and the first flow cell device during one or more of: (f)-(i).

In some embodiments, the sequencing method further comprising: repeating operations (a)-(e). Each repetition of operation (a) to (e) can occur within a single cycle or across different flow cycles of a sequence run. In some embodiments, the sequencing method further includes repeating operations (f)-(j). Each repetition of operation (f) to (j) can occur within a single cycle or across different flow cycles of a sequence run. Each repetition of operation (f) to (j) can occur after operations (a) to (e).

In some embodiments, the sequencing method further includes repeating operations (a)-(j) for a number of repetitions for a nest bank holding two different flow cell devices. Similar operations can also be repeated for the number of repetition with respect to additional flow cell devices that the nest bank is configured to hold. In some embodiments, the number of repetitions is in a range from 1 to 500, inclusive of all ranges and subranges therebetween. In some embodiments, the number of repetitions corresponds to the number of cycles within a sequence run.

In some embodiments, allowing fluidic communication in operation (f) between the nest bank and the first flow cell device may include reversibly fastening the flow cell device (e.g., flow cell device 200) to a carrier (e.g., carrier 2051) via one or more fasteners to enable sealed fluidic communication between the flow cell device and the carrier; and reversibly fastening the carrier to the nest bank via the one or more fasteners to enable sealed fluidic communication between the nest bank and the carrier and to enable physical contact to heat dissipation elements. The one or more fasteners between the carrier and the flow cell device may include screw, pins, mechanical clamps, or other structure with magnetic forces.

In some embodiments, operation (a) moving the first flow cell device from the nest bank to the x-y stage is within a first flow cycle of a sequence run and operation (f) moving the first flow cell device from the nest bank to the x-y stage is within a second flow cycle different from the first flow cycle. In some embodiments, operation (f) simultaneously allowing fluidic and thermal communication between the nest bank and the first flow cell device during one or more of: (a)-(e) includes turning the one or more fasteners into an on-stage to enable sealed fluidic communication and physical contact for thermal communication. For example, switch on the electric power supply to an electromagnetic coil.

In some embodiments, the operation (f) simultaneously allowing fluidic and thermal communication between the nest bank and the first flow cell device during one or more of: (a)-(e) may include dipping the first flow cell device into at least some of the one or more reagent containers in a predetermined sequence.

In some embodiments, each of the operations of: (a)-(b) and (d)-(g) is completed within less than 0.5 seconds, 1 second, 2 seconds, 3 seconds, 4 seconds, 5 seconds, 6 seconds, 7 seconds, 8 seconds, 9 seconds, or 10 seconds. In some embodiments, each of the operations of: (a)-(b) and (d)-(g) is completed within less than 0.5 seconds, 1 second, 2 seconds, or 3 seconds.

### Computer Systems

Various embodiments of the methods may be implemented, for example, using one or more computer systems, such as computer system 800 shown in FIG. 8. One or more computer systems 800 may be used, for example, to implement any of the embodiments discussed herein, as well as combinations and sub-combinations thereof.

Computer system 800 may include one or more hardware processors 404. The hardware processor 804 can be central processing unit (CPU), graphic processing units (GPU), or their combination. Processor 804 may be connected to a bus or communication infrastructure 806.

Computer system 800 may also include user input/output device(s) 803, such as monitors, keyboards, pointing devices, etc., which may communicate with communication infrastructure 406 through user input/output interface(s) 802. The user input/output devices 803 may be coupled to the user interface 124 in FIG. 1.

One or more of processors 804 may be a graphics processing unit (GPU). In an embodiment, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, vector processing, array processing, etc., as well as cryptography (including brute-force cracking), generating cryptographic hashes or hash sequences, solving partial hash-inversion problems, and/or producing results of other proof-of-work computations for some blockchain-based applications, for example. With capabilities of general-purpose computing on graphics processing units (GPGPU), the GPU may be particularly useful in at least the image recognition and machine learning aspects described herein.

Additionally, one or more of processors 404 may include a coprocessor or other implementation of logic for accelerating cryptographic calculations or other specialized mathematical functions, including hardware-accelerated cryptographic coprocessors. Such accelerated processors may further include instruction set(s) for acceleration using coprocessors and/or other logic to facilitate such acceleration.

The computer system 800 may also include a data storage device such as a main or primary memory 808, e.g., random access memory (RAM). Main memory 808 may include one or more levels of cache. Main memory 808 may have stored therein control logic (i.e., computer software) and/or data.

The computer system 800 may also include one or more secondary data storage devices or secondary memory 810. Secondary memory 810 may include, for example, a main storage drive 812 and/or a removable storage device or drive 814. Main storage drive 812 may be a hard disk drive or solid-state drive, for example. Removable storage drive 814 may be a floppy disk drive, a magnetic tape drive, a compact disk drive, an optical storage device, tape backup device, and/or any other storage device/drive.

The removable storage drive 814 may interact with a removable storage unit 818.

The removable storage unit 818 may include a computer usable or readable storage device having stored thereon computer software and/or data. The software can include control logic. The software may include instructions executable by the hardware processor(s) 804. Removable storage unit 818 may be a floppy disk, magnetic tape, compact disk, DVD, optical storage disk, and/any other computer data storage device. Removable storage drive 814 may read from and/or write to removable storage unit 818.

The secondary memory 810 may include other means, devices, components, instrumentalities or other approaches for allowing computer programs and/or other instructions and/or data to be accessed by computer system 800. Such means, devices, components, instrumentalities or other approaches may include, for example, a removable storage unit 822 and an interface 820. Examples of the removable storage unit 822 and the interface 820 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

The computer system 800 may further include a communication or network interface 824. The communication interface 824 may enable computer system 800 to communicate and interact with any combination of external devices, external networks, external entities, etc. (individually and collectively referenced by reference number 828). For example, the communication interface 824 may allow computer system 800 to communicate with external or remote devices 828 over communication path 826, which may be wired and/or wireless (or a combination thereof), and which may include any combination of LANs, WANs, the Internet, etc. Control logic and/or data may be transmitted to and from computer system 800 via communication path 826. In some embodiments, communication path 826 is the connection to the cloud 130, as depicted in FIG. 1. The external devices, etc. referred to by reference number 828 may be devices, networks, entities, etc. in the cloud 130.

The computer system 800 may also be any of a personal digital assistant (PDA), desktop workstation, laptop or notebook computer, netbook, tablet, smart phone, smart watch or other wearable, appliance, part of the Internet of Things (IoT), and/or embedded system, to name a few non-limiting examples, or any combination thereof.

It should be appreciated that the framework described herein may be implemented as a method, process, apparatus, system, or article of manufacture such as a non-transitory computer-readable medium or device. For illustration purposes, the present framework may be described in the context of distributed ledgers being publicly available, or at least available to untrusted third parties. One example as a modern use case is with blockchain-based systems. It should be appreciated, however, that the present framework may also be applied in other settings where sensitive or confidential information may need to pass by or through hands of untrusted third parties, and that this technology is in no way limited to distributed ledgers or blockchain uses.

The computer system 800 may be a client or server, accessing or hosting any applications and/or data through any delivery paradigm, including but not limited to remote or distributed cloud computing solutions; local or on-premises software (e.g., “on-premise” cloud-based solutions); “as a service” models (e.g., content as a service (CaaS), digital content as a service (DCaaS), software as a service (Saas), managed software as a service (MSaaS), platform as a service (PaaS), desktop as a service (DaaS), framework as a service (FaaS), backend as a service (BaaS), mobile backend as a service (MBaaS), infrastructure as a service (IaaS), database as a service (DBaaS), etc.); and/or a hybrid model including any combination of the foregoing examples or other services or delivery paradigms.

Any applicable data structures, file formats, and schemas may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

Any pertinent data, files, and/or databases may be stored, retrieved, accessed, and/or transmitted in human-readable formats such as numeric, textual, graphic, or multimedia formats, further including various types of markup language, among other possible formats. Alternatively, or in combination with the above formats, the data, files, and/or databases may be stored, retrieved, accessed, and/or transmitted in binary, encoded, compressed, and/or encrypted formats, or any other machine-readable formats.

Interfacing or interconnection among various systems and layers may employ any number of mechanisms, such as any number of protocols, programmatic frameworks, floorplans, or application programming interfaces (API), including but not limited to Document Object Model (DOM), Discovery Service (DS), NSUserDefaults, Web Services Description Language (WSDL), Message Exchange Pattern (MEP), Web Distributed Data Exchange (WDDX), Web Hypertext Application Technology Working Group (WHATWG) HTML5 Web Messaging, Representational State Transfer (REST or RESTful web services), Extensible User Interface Protocol (XUP), Simple Object Access Protocol (SOAP), XML Schema Definition (XSD), XML Remote Procedure Call (XML-RPC), or any other mechanisms, open or proprietary, that may achieve similar functionality and results.

Such interfacing or interconnection may also make use of uniform resource identifiers (URI), which may further include uniform resource locators (URL) or uniform resource names (URN). Other forms of uniform and/or unique identifiers, locators, or names may be used, either exclusively or in combination with forms such as those set forth above.

Any of the above protocols or APIs may interface with or be implemented in any programming language, procedural, functional, or object-oriented, and may be compiled or interpreted. Non-limiting examples include C, C++, C #, Objective-C, Java, Scala, Clojure, Elixir, Swift, Go, Perl, PHP, Python, Ruby, JavaScript, WebAssembly, or virtually any other language, with any other libraries or schemas, in any kind of framework, runtime environment, virtual machine, interpreter, stack, engine, or similar mechanism, including but not limited to Node.js, V8, Knockout, jQuery, Dojo, Dijit, OpenUI5, AngularJS, Expressjs, Backbone.js, Ember.js, DHTMLX, Vue, React, Electron, and so on, among many other non-limiting examples.

In some embodiments, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 800, main memory 808, secondary memory 810, and removable storage units 818 and 822, as well as tangible articles of manufacture embodying any combination of the foregoing. Such control logic, when executed by one or more data processing devices (such as computer system 800), may cause such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 8. In particular, embodiments may operate with software, hardware, and/or operating system implementations other than those described herein.

### Imaging Systems

The imager 116 in FIG. 1 can include one or more optical systems 2020. Further disclosed herein are optical system design guidelines and high-performance fluorescence imaging methods and systems that provide improved optical resolution and image quality for fluorescence imaging-based genomics applications. The disclosed optical imaging system designs provide for larger fields-of-view, increased spatial resolution, improved modulation transfer, contrast-to-noise ratio, and image quality, higher spatial sampling frequency, faster transitions between image capture when repositioning the sample plane to capture a series of images (e.g., of different fields-of-view), and improved imaging system duty cycle, and thus enable higher throughput image acquisition and analysis.

In some instances, improvements in imaging performance, e.g., for dual-side (flow cell) imaging applications, may be achieved by using an electro-optical phase plate in combination with an objective lens to compensate for the optical aberrations induced by the layer of fluid separating the upper (near) and lower (far) interior surfaces of a flow cell. In some instances, this design approach may also compensate for vibrations introduced by, e.g., a motion-actuated compensator that is moved in or out of the optical path depending on which surface of the flow cell is being images.

In some instances, improvements in imaging performance, e.g., for dual-side (flow cell) imaging applications comprising the use of thick flow cell walls (e.g., wall (or coverslip) thickness>700 μm) and fluid channels (e.g., fluid channel height or thickness of 50-200 μm) may be achieved even when using commercially-available, off-the-shelf objectives by using a tube lens design that corrects for the optical aberrations induced by the thick flow cell walls and/or intervening fluid layer in combination with the objective.

In some instances, improvements in imaging performance, e.g., for multichannel (e.g., two-color or four-color) imaging applications, may be achieved by using multiple tube lenses, one for each imaging channel, where each tube lens design has been optimized for the specific wavelength range used in that imaging channel.

Exemplary embodiments disclosed herein may comprise fluorescence imaging systems, said systems comprising: a) at least one light source configured to provide excitation light within one or more specified wavelength ranges; b) an objective lens configured to collect fluorescence arising from within a specified field-of-view of a sample plane upon exposure of the sample plane to the excitation light, wherein a numerical aperture of the objective lens is at least 0.1, at least 0.2, at least 0.3, at least 0.4, at least 0.5, at least 0.6, at least 0.7, at least 0.8, or at least 0.9 or a numerical aperture value falling within a range defined by any two of the foregoing; wherein a working distance of the objective lens is at least 400 micron (μm), at least 500 μm, at least 600 μm, at least 700 μm, at least 800 μm, at least 900 μm, at least 1000 μm, or a working distance falling within a range defined by any two of the foregoing; and wherein the field-of-view has an area of at least 0.1 mm², at least 0.2 mm², at least 0.5 mm², at least 0.7 mm², at least 1 mm², at least 2 mm², at least 3 mm², at least 5 mm², or at least 10 mm², or a field of view falling within a range defined by any two of the foregoing; and c) at least one image sensor, wherein the fluorescence collected by the objective lens is imaged onto the image sensor, and wherein a pixel dimension for the image sensor is chosen such that a spatial sampling frequency for the fluorescence imaging system is at least twice an optical resolution of the fluorescence imaging system.

In some embodiments, the numerical aperture may be at least 0.75. In some embodiments, the numerical aperture is at least 1.0. In some embodiments, the working distance is at least 850 μm. In some embodiments, the working distance is at least 1,000 μm. In some embodiments, the working distance is between 500 μm and 7,000 μm, between 100 μm and 5,000 μm, or between 500 μm and 2,000 μm. In some embodiments, the field-of-view may have an area of at least 2.5 mm². In some embodiments, the field-of-view may have an area of at least 3 mm². In some embodiments, the field-of-view may have an area of between 0.5 mm² and 10 mm², between 1 mm² and 10 mm², between 1 mm² and 5 mm², between 1.5 mm² and 5 mm², between 2 mm² and 5 mm², or between 2.5 mm² and 3 mm². In some embodiments, the spatial sampling frequency may be at least 2.5 times the optical resolution of the fluorescence imaging system. In some embodiments, the spatial sampling frequency may be at least 3 times the optical resolution of the fluorescence imaging system. In some embodiments, the spatial sampling frequency is between 2 and 10 times the optical resolution, between 2 and 5 times the optical resolution, or between 2.5 and 3 times the optical resolution. In some embodiments, the system may further comprise an X-Y-Z translation stage such that the system is configured to acquire a series of two or more fluorescence images in an automated fashion, wherein each image of the series is or can be acquired for a different field-of-view. In some embodiments, a position of the sample plane may be simultaneously adjusted in an X direction, a Y direction, and a Z direction to match the position of an objective lens focal plane in between acquiring images for different fields-of-view. In some embodiments, the time required for the simultaneous adjustments in the X direction, Y direction, and Z direction may be less than 0.3 seconds, less than 0.4 seconds, less than 0.5 seconds, less than 0.7 seconds, or less than 1 second, or a time falling within a range defined by any two of the foregoing. In some embodiments, the system further comprises an autofocus mechanism configured to adjust the focal plane position prior to acquiring an image of a different field-of-view if an error signal indicates that a difference in the position of the focal plane and the sample plane in the Z direction is greater than a specified error threshold. In some embodiments, the specified error threshold is 100 nm or greater. In some embodiments, the specified error threshold is 50 nm or less. In some embodiments, the system comprises three or more image sensors, and wherein the system is configured to image fluorescence in each of three or more wavelength ranges onto a different image sensor. In some embodiments, a difference in the position of a focal plane for each of the three or more image sensors and the sample plane is less than 100 nm. In some embodiments, a difference in the position of a focal plane for each of the three or more image sensors and the sample plane is less than 50 nm. In some embodiments, the total time required to reposition the sample plane, adjust focus if necessary, and acquire an image is less than 0.4 seconds per field-of-view. In some embodiments, the total time required to reposition the sample plane, adjust focus if necessary, and acquire an image is less than 0.3 seconds per field-of-view.

Also discloser herein are fluorescence imaging systems for dual-side imaging of a flow cell comprising: a) an objective lens configured to collect fluorescence arising from within a specified field-of-view of a sample plane within the flow cell; b) at least one tube lens positioned between the objective lens and at least one image sensor, wherein the at least one tube lens is configured to correct an imaging performance metric for a combination of the objective lens, the at least one tube lens, and the at least one image sensor when imaging an interior surface of the flow cell, and wherein the flow cell has a wall thickness of at least 700 μm (for example, between 500 μm and 3,000 μm, between 700 μm and 2,000 μm, between 700 and 1,500 μm, between 1,000 μm and 2,000 , between 2,000 μm and 3,000 μm or any range therebetween) and a gap between an upper interior surface and a lower interior surface of at least 50 μm (for example, between 50 μm and 1,000 μm, between 50 and 200 μm, between 100 μm and 500 μm, between 100 μm and 200 μm, or any range therebetween); wherein the imaging performance metric is substantially the same for imaging the upper interior surface or the lower interior surface of the flow cell without moving an optical compensator into or out of an optical path between the flow cell and the at least one image sensor, without moving one or more optical elements of the tube lens along the optical path, and without moving one or more optical elements of the tube lens into or out of the optical path.

In some embodiments, the objective lens may be a commercially-available microscope objective lens. Selection of a suitable objective lens will be within the knowledge of the person of ordinary skill in the art. In some embodiments, the commercially-available microscope objective may have a numerical aperture of at least 0.3. In some embodiments, the objective lens may have a working distance of at least 700 μm. In some embodiments, the objective lens may be corrected to compensate for a cover slip thickness (or flow cell wall thickness) of 0.17 mm or of greater or lesser thickness than 0.17 mm. In some embodiments, the optical system may be corrected to compensate for cover slip thickness, flow cell thickness, or distance between desired focal planes. In some embodiments, said correction may be made by inserting a corrective optic, such as a lens or optical assembly into the light path of the optical system. In some embodiments, said correction may be made without inserting a corrective optic, such as a lens or optical assembly into the light path of the optical system. In some embodiments, the fluorescence imaging system may further comprise an electro-optical phase plate positioned adjacent to the objective lens and between the objective lens and the tube lens, wherein the electro-optical phase plate may provide correction for optical aberrations caused by a fluid filling the gap between the upper interior surface and the lower interior surface of the flow cell. In some embodiments, the at least one tube lens may be a compound lens comprising three or more optical components. In some embodiments, the at least one tube lens is a compound lens comprising four optical components, which may comprise one or more of a first asymmetric convex-convex lens, a second convex-plano lens, a third asymmetric concave-concave lens, and a fourth asymmetric convex-concave lens which may be present in the order as listed above, or in any alternate order. In some embodiments, the at least one tube lens is configured to correct an imaging performance metric for a combination of the objective lens, the at least one tube lens, and the at least one image sensor when imaging an interior surface of a flow cell having a wall thickness of at least 1 mm. In some embodiments, the at least one tube lens is configured to correct an imaging performance metric for a combination of the objective lens, the at least one tube lens, and the at least one image sensor when imaging an interior surface of a flow cell having a gap of at least 100 μm. In some embodiments, the at least one tube lens is configured to correct an imaging performance metric for a combination of the objective lens, the at least one tube lens, and the at least one image sensor when imaging an interior surface of a flow cell having a gap of at least 200 μm. In some embodiments, the system comprises a single objective lens, two tube lenses, and two image sensors, and each of the two tube lenses is designed to provide optimal imaging performance at a different fluorescence wavelength. In some embodiments, the system comprises a single objective lens, three tube lenses, and three image sensors, and each of the three tube lenses is designed to provide optimal imaging performance at a different fluorescence wavelength. In some embodiments, the system comprises a single objective lens, four tube lenses, and four image sensors, and each of the four tube lenses is designed to provide optimal imaging performance at a different fluorescence wavelength. In some embodiments, the design of the objective lens or the at least one tube lens is configured to optimize the modulation transfer function in the mid to high spatial frequency range. In some embodiments, the imaging performance metric comprises a measurement of modulation transfer function (MTF) at one or more specified spatial frequencies, defocus, spherical aberration, chromatic aberration, coma, astigmatism, field curvature, image distortion, contrast-to-noise ratio (CNR), or any combination thereof. In some embodiments, the difference in the imaging performance metric for imaging the upper interior surface and the lower interior surface of the flow cell is less than 10%. In some embodiments, the difference in imaging performance metric for imaging the upper interior surface and the lower interior surface of the flow cell is less than 5%. In some embodiments, the use of the at least one tube lens provides for an at least equivalent or better improvement in the imaging performance metric for dual-side imaging compared to that for a conventional system comprising an objective lens, a motion-actuated compensator, and an image sensor. In some embodiments, the use of the at least one tube lens provides for an at least 10% improvement in the imaging performance metric for dual-side imaging compared to that for a conventional system comprising an objective lens, a motion-actuated compensator, and an image sensor.

Disclosed herein are illumination systems for use in imaging-based solid-phase genotyping and sequencing applications, the illumination system comprising: a) a light source; and b) a liquid light-guide configured to collect light emitted by the light source and deliver it to a specified field-of-illumination on a support surface comprising tethered biological macromolecules.

In some embodiments, the illumination system further comprises a condenser lens. In some embodiments, the specified field-of-illumination has an area of at least 2 mm². In some embodiments, the light delivered to the specified field-of-illumination is of uniform intensity across a specified field-of-view for an imaging system used to acquire images of the support surface. In some embodiments, the specified field-of-view has an area of at least 2 mm². In some embodiments, the light delivered to the specified field-of-illumination is of uniform intensity across the specified field-of-view when a coefficient of variation (CV) for light intensity is less than 10%. In some embodiments, the light delivered to the specified field-of-illumination is of uniform intensity across the specified field-of-view when a coefficient of variation (CV) for light intensity is less than 5%. In some embodiments, the light delivered to the specified field-of-illumination has a speckle contrast value of less than 0.1. In some embodiments, the light delivered to the specified field-of-illumination has a speckle contrast value of less than 0.05.

It will be understood by those of skill in the art that the disclosed optical systems, imaging systems, or modules may, in some instances, be stand-alone optical systems designed for imaging a sample or substrate surface. In some instances, they may comprise one or more processors or computers. In some instances, they may comprise one or more software packages that provide instrument control functionality and/or image processing functionality. In some instances, in addition to optical components such as light sources (e.g., solid-state lasers, dye lasers, diode lasers, arc lamps, tungsten-halogen lamps, etc.), lenses, prisms, mirrors, dichroic reflectors, optical filters, optical bandpass filters, apertures, and image sensors (e.g., complementary metal oxide semiconductor (CMOS) image sensors and cameras, charge-coupled device (CCD) image sensors and cameras, etc.), they may also include mechanical and/or optomechanical components, such as an X-Y translation stage, an X-Y-Z translation stage, a piezoelectric focusing mechanism, and the like. In some instances, they may function as modules, components, sub-assemblies, or sub-systems of larger systems designed for genomics applications (e.g., genetic testing and/or nucleic acid sequencing applications). For example, in some instances, they may function as modules, components, sub-assemblies, or sub-systems of larger systems that further comprise light-tight and/or other environmental control housings, temperature control modules, fluidics control modules, fluid dispensing robotics, pick-and-place robotics, one or more processors or computers, one or more local and/or cloud-based software packages (e.g., instrument/system control software packages, image processing software packages, data analysis software packages), data storage modules, data communication modules (e.g., Bluetooth, WiFi, intranet, or internet communication hardware and associated software), display modules, or any combination thereof.

### Methods for Sequencing

The present disclosure provides methods for sequencing immobilized or non-immobilized nucleic acid template molecules. The methods can be operated in the sequencing system 110, for example, in the sequencer 114. In some embodiments, the immobilized nucleic acid template molecules comprise a plurality of nucleic acid template molecules having one copy of a target sequence of interest. In some embodiments, nucleic acid template molecules having one copy of a target sequence of interest can be generated by conducting bridge amplification using linear library molecules. In some embodiments, the immobilized nucleic acid template molecules comprise a plurality of nucleic acid template molecules each having two or more tandem copies of a target sequence of interest (e.g., concatemer template molecules). In some embodiments, the nucleic acid template molecules comprising concatemer template molecules can be generated by conducting rolling circle amplification of circularized library molecules. In some embodiments, the non-immobilized nucleic acid template molecules comprise circular molecules. The methods for sequencing can employ soluble (e.g., non-immobilized) sequencing polymerases or sequencing polymerases that are immobilized to a support. The person of ordinary skill in the art will be able to select polymerases suitable for the various sequencing methods disclosed herein.

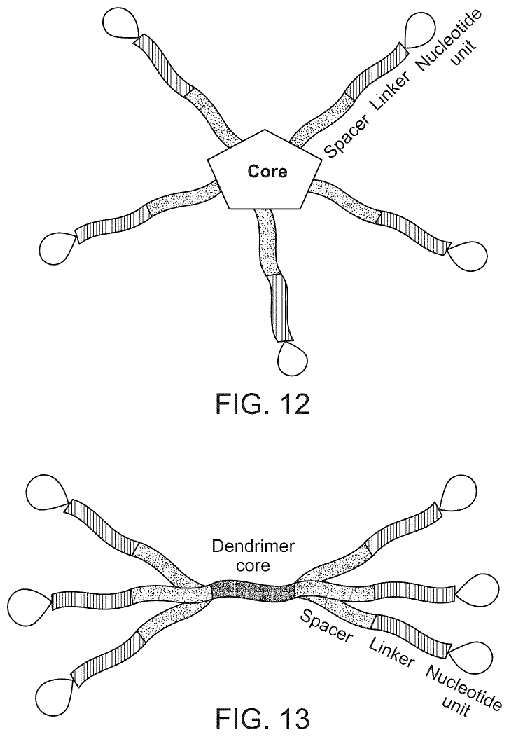

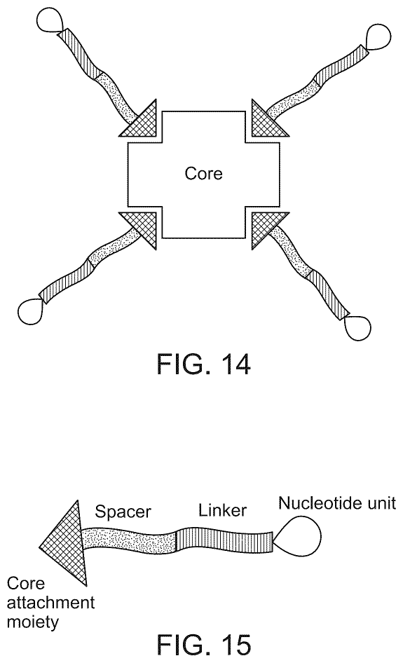

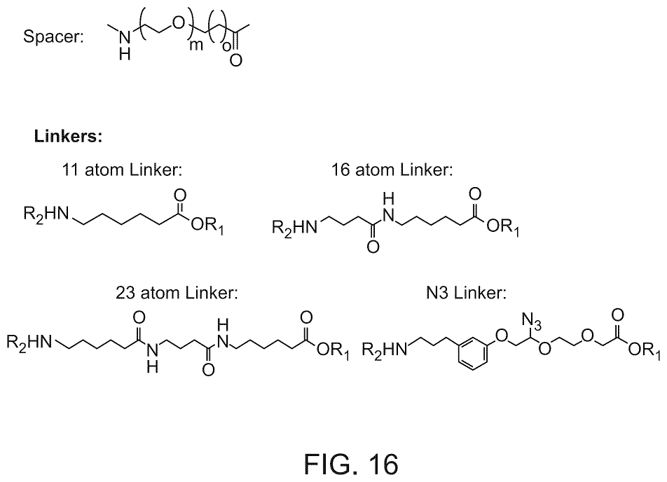

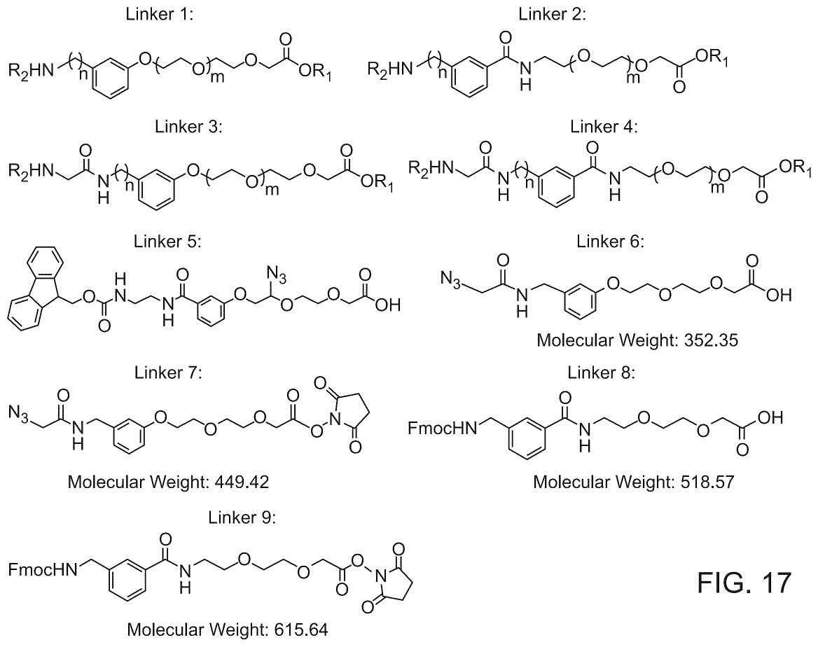

In some embodiments, the sequencing reactions employ detectably labeled nucleotide analogs. In some embodiments, the sequencing reactions employ a two-stage sequencing reaction comprising a first stage of binding detectably labeled multivalent molecules (see FIGS. 11-15), and a second stage of incorporating nucleotide analogs, described in further detail below. In some embodiments, the sequencing reactions employ non-labeled nucleotide analogs. In some embodiments, the sequencing reactions employ phosphate chain labeled nucleotides.

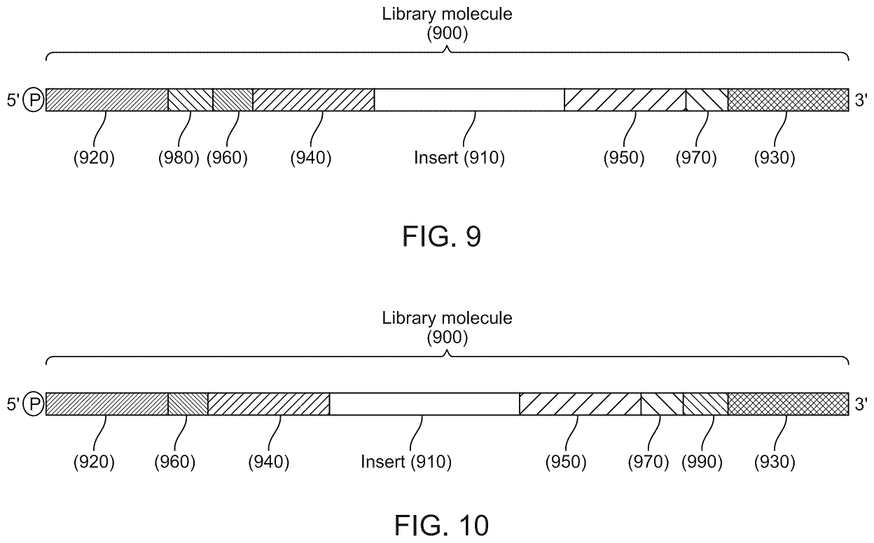

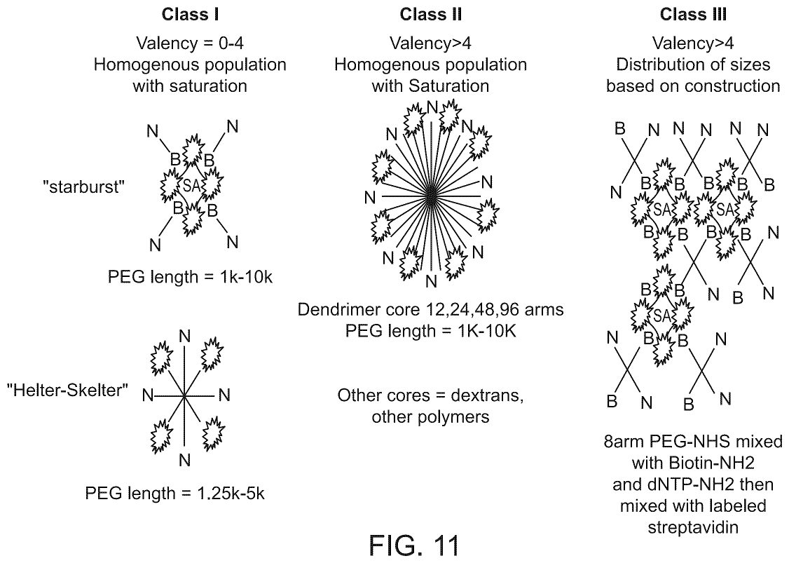

In some embodiments, the immobilized concatemer template molecules comprise tandem repeat units of the sequence-of-interest (also referred to as the insert region) and any adaptor sequences. For example, the tandem repeat unit comprises: (i) a left universal adaptor sequence having a binding sequence for a first surface primer (920) (e.g., surface pinning primer), (ii) a left universal adaptor sequence having a binding sequence for a first sequencing primer (940) (e.g., forward sequencing primer), (iii) a sequence-of-interest (910), (iv) a right universal adaptor sequence having a binding sequence for a second sequencing primer (950) (e.g., reverse sequencing primer), (v) a right universal adaptor sequence having a binding sequence for a second surface primer (930) (e.g., surface capture primer), and (vii) a left index sequence (960) and/or a right index sequence (070), which can be sample index sequences. In some embodiments, the tandem repeat unit further comprises a left unique identification sequence (980) and/or a right unique identification sequence (990). In some embodiments, the tandem repeat unit further comprises at least one binding sequence for a compaction oligonucleotide. FIGS. 9 and 10 show exemplary embodiments of linear library molecules or single units of a concatemer template molecule.

The immobilized concatemer template molecule can self-collapse into a compact nucleic acid nanoball. Inclusion of one or more compaction oligonucleotides, with binding sites on the concatemer template molecule, during the RCA reaction can further compact the size and/or shape of the nanoball. An increase in the number of tandem repeat units in each concatemer template molecule increases the number of sites along the concatemer template molecule for hybridizing to multiple sequencing primers (e.g., sequencing primers having a universal sequence) which serve as multiple initiation sites for polymerase-catalyzed sequencing reactions. When the sequencing reaction employs detectably labeled nucleotides and/or detectably labeled multivalent molecules (e.g., multivalent molecules having nucleotide units), the signals emitted by the nucleotides or nucleotide units that participate in the parallel sequencing reactions along the concatemer template molecule yield an increased signal intensity for each concatemer template molecule. Multiple portions of a given concatemer template molecule can be simultaneously sequenced. Furthermore, a plurality of binding complexes can form along a particular concatemer template molecule, each binding complex comprising a sequencing polymerase bound to a template/primer duplex and a multivalent molecule, wherein the plurality of binding complexes remain stable without dissociation resulting in increased persistence time which increases signal intensity and reduces imaging time.

### Methods for Sequencing Using Nucleotide Analogs

The present disclosure provides methods for sequencing any of the immobilized template molecules described herein, the methods comprising step (a): contacting a sequencing polymerase with (i) a nucleic acid template molecule and (ii) a nucleic acid sequencing primer, wherein the contacting is conducted under a condition suitable to bind the sequencing polymerase to the nucleic acid template molecule which is hybridized to the nucleic acid primer, wherein the nucleic acid template molecule hybridized to the nucleic acid primer forms the nucleic acid duplex. In some embodiments, the sequencing polymerase comprises a recombinant mutant sequencing polymerase that can bind and incorporate nucleotide analogs. Exemplary polymerases are described, for example, in U.S. Pat. No. 11,891,241, the contents of which are incorporated by reference in their entirety herein.

In some embodiments of the sequencing methods, the sequencing primer, e.g. the first and/or second sequencing primer, comprises a 3′ extendible end or a 3′ non-extendible end. In some embodiments, the plurality of nucleic acid template molecules comprise amplified template molecules (e.g., clonally amplified template molecules). In some embodiments, the plurality of nucleic acid template molecules comprise one copy of a target sequence of interest. In some embodiments, the plurality of nucleic acid molecules comprise two or more tandem copies of a target sequence of interest (e.g., concatemer template molecules). In some embodiments, the plurality of nucleic acid template molecules comprise the same sequence of interest. In some embodiments, individual nucleic acid template molecules in the plurality comprise different sequences of interest. In some embodiments, the plurality of nucleic acid primers are in solution or are immobilized to a support, e.g. the support 210 of the flow cell devices disclosed herein. In some embodiments, when the plurality of nucleic acid template molecules and/or the plurality of nucleic acid primers are immobilized to the support, binding with the first sequencing polymerases generates a plurality of immobilized first complexed polymerases. In some embodiments, the plurality of nucleic acid template molecules and/or nucleic acid primers are immobilized to 10²-10¹⁵ different sites on a support. In some embodiments, binding of the plurality of template molecules and nucleic acid primers with the plurality of first sequencing polymerases generates a plurality of first complexed polymerases immobilized to 10²-10¹⁵ different sites on the support. In some embodiments, the plurality of immobilized first complexed polymerases on the support are immobilized to pre-determined or to random sites on the support. In some embodiments, the plurality of immobilized first complexed polymerases are in fluid communication with each other to permit flowing a solution of reagents using the sequencing system 110 described herein (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

In some embodiments, the methods for sequencing further comprise step (b): contacting the sequencing polymerase with a plurality of nucleotides under a condition suitable for binding at least one nucleotide to the sequencing polymerase which is bound to the nucleic acid duplex and suitable for polymerase-catalyzed nucleotide incorporation which extends the sequencing primer by one nucleotide. In some embodiments, the sequencing polymerase is contacted with the plurality of nucleotides in the presence of at least one catalytic cation comprising magnesium and/or manganese. In some embodiments, the plurality of nucleotides comprises at least one nucleotide analog having a chain terminating moiety at the sugar 2′ or 3′ position. In some embodiments, the chain terminating moiety is removable from the sugar 2′ or 3′ position to convert the chain terminating moiety to an OH or H group. In some embodiments, the plurality of nucleotides comprises at least one nucleotide that lacks a chain terminating moiety. In some embodiments, at least on nucleotide in the plurality is labeled with a detectable reporter moiety (e.g., fluorophore) that emits a detectable signal. In some embodiments, detectable reporter moiety comprises a fluorophore. In some embodiments, the fluorophore is attached to the nucleo-base. In some embodiments, the fluorophore is attached to the nucleo-base with a linker which is cleavable/removable from the nucleo-base. In some embodiments, at least one of the nucleotides in the plurality is not labeled with a detectable reporter moiety. In some embodiments, a particular detectable reporter moiety (e.g., fluorophore) that is attached to the nucleotide can correspond to the identity of nucleo-base (e.g., dATP, dGTP, dCTP, dTTP or dUTP) and therefore the corresponding complementary nucleo-base in the nucleic acid template molecule to permit detection and identification of the nucleo-base in the nucleic acid template molecule. When the incorporated chain terminating nucleotide is detectably labeled, step (b) can further comprise detecting the emitted signal from the incorporated chain terminating nucleotide. In some embodiments, step (b) further comprises identifying the nucleo-base of the incorporated chain terminating nucleotide.

In some embodiments, the methods for sequencing further comprise step (c): removing the chain terminating moiety from the incorporated chain terminating nucleotide to generate an extendible 3′ OH group. In some embodiments, step (c) further comprises removing the detectable label from the incorporated chain terminating nucleotide. In some embodiments, the sequencing polymerase remains bound to the template molecule which is hybridized to the sequencing primer which is extended by one nucleo-base.

In some embodiments, the methods for sequencing further comprise step (d): repeating steps (b) and (c) at least once.

### Two-Stage Methods for Nucleic Acid Sequencing

The present disclosure provides a two-stage method for sequencing any of the immobilized template molecules described herein. In some embodiments, the first stage generally comprises binding multivalent molecules to complexed polymerases to form multivalent-complexed polymerases, and detecting the multivalent-complexed polymerases.

In some embodiments, the first stage comprises step (a): contacting a plurality of a first sequencing polymerase to (i) a plurality of nucleic acid template molecules and (ii) a plurality of nucleic acid sequencing primers, wherein the contacting is conducted under a condition suitable to bind the plurality of first sequencing polymerases to the plurality of nucleic acid template molecules and the plurality of nucleic acid primers thereby forming a plurality of first complexed polymerases each comprising a first sequencing polymerase bound to a nucleic acid duplex wherein the nucleic acid duplex comprises a nucleic acid template molecule hybridized to a nucleic acid primer. In some embodiments, the first polymerase comprises a recombinant mutant sequencing polymerase.

In some embodiments, the sequencing primer comprises an oligonucleotide having a 3′ extendible end or a 3′ non-extendible end. In some embodiments, the plurality of nucleic acid template molecules comprise amplified template molecules (e.g., clonally amplified template molecules). In some embodiments, the plurality of nucleic acid template molecules comprise one copy of a sequence of interest. In some embodiments, the plurality of nucleic acid molecules comprise two or more tandem copies of a sequence of interest (e.g., concatemer template molecules). In some embodiments, the nucleic acid template molecules in the plurality of nucleic acid template molecules comprise the same sequence of interest. In some embodiments, individual nucleic acid template molecules in the plurality comprise different sequences of interest. In some embodiments, the plurality of nucleic acid template molecules and/or the plurality of nucleic acid primers are in solution or are immobilized to a support, e.g. the support 210 of the flow cell devices disclosed herein. In some embodiments, when the plurality of nucleic acid template molecules and/or the plurality of nucleic acid primers are immobilized to a support, the binding with the first sequencing polymerase generates a plurality of immobilized first complexed polymerases. In some embodiments, the plurality of nucleic acid template molecules and/or nucleic acid primers are immobilized to 10²-10¹⁵ different sites on a support. In some embodiments, the binding of the plurality of template molecules and nucleic acid primers with the plurality of first sequencing polymerases generates a plurality of first complexed polymerases immobilized to 10²-10¹⁵ different sites on the support. In some embodiments, the plurality of immobilized first complexed polymerases on the support are immobilized to pre-determined or to random sites on the support. In some embodiments, the plurality of immobilized first complexed polymerases are in fluid communication with each other to permit flowing a solution of reagents using the sequencing system 110 described herein (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

In some embodiments, the methods for sequencing further comprise step (b): contacting the plurality of first complexed polymerases with a plurality of multivalent molecules to form a plurality of multivalent-complexed polymerases (e.g., binding complexes). In some embodiments, individual multivalent molecules in the plurality of multivalent molecules comprise a core attached to multiple nucleotide arms. In some embodiments, each nucleotide arm is attached to a nucleotide (e.g., nucleotide unit) (e.g., FIGS. 11-15). In some embodiments, the contacting of step (b) is conducted under a condition suitable for binding complementary nucleotide units of the multivalent molecules to at least two of the plurality of first complexed polymerases thereby forming a plurality of multivalent-complexed polymerases. In some embodiments, the condition is suitable for inhibiting polymerase-catalyzed incorporation of the complementary nucleotide units into the primers of the plurality of multivalent-complexed polymerases. In some embodiments, the plurality of multivalent molecules comprise at least one multivalent molecule having multiple nucleotide arms (e.g., FIGS. 9-14) each attached with a nucleotide analog (e.g., nucleotide analog unit), where the nucleotide analog includes a chain terminating moiety at the sugar 2′ and/or 3′ position. In some embodiments, the plurality of multivalent molecules comprises at least one multivalent molecule comprising multiple nucleotide arms each attached with a nucleotide unit that lacks a chain terminating moiety. In some embodiments, at least one of the multivalent molecules in the plurality of multivalent molecules is labeled with a detectable reporter moiety that emits a signal. In some embodiments, the detectable reporter moiety comprises a fluorophore. In some embodiments, the contacting of step (b) is conducted in the presence of at least one non-catalytic cation comprising strontium, barium and/or calcium.

In some embodiments, the methods for sequencing further comprise step (c): detecting the plurality of multivalent-complexed polymerases. In some embodiments, the detecting includes detecting the signals emitted by the multivalent molecules that are bound to the complexed polymerases, where the complementary nucleotide units of the multivalent molecules are bound to the primers but incorporation of the complementary nucleotide units is inhibited. In some embodiments, the multivalent molecules are labeled with a detectable reporter moiety to permit detection. In some embodiments, the labeled multivalent molecules comprise a fluorophore attached to the core, linker and/or nucleotide unit of the multivalent molecules.

In some embodiments, the methods for sequencing further comprise step (d): identifying the nucleo-base of the complementary nucleotide units that are bound to the plurality of first complexed polymerases, thereby determining the identity of the corresponding nucleo-base in the nucleic acid template molecule, and thus the sequence of the nucleic acid template molecule. In some embodiments, the multivalent molecules are labeled with a detectable reporter moiety that corresponds to the particular nucleotide units attached to the nucleotide arms to permit identification of the complementary nucleotides in the nucleic acid molecule (e.g., nucleotide base adenine, guanine, cytosine, thymine or uracil) that are bound to the plurality of first complexed polymerases.

In some embodiments, the methods for sequencing further comprise step (e): dissociating the plurality of multivalent-complexed polymerases and removing the plurality of first sequencing polymerases and their bound multivalent molecules, and retaining the plurality of nucleic acid duplexes.

The second stage of the two-stage sequencing method generally comprises nucleotide incorporation. In some embodiments, the methods for sequencing further comprises step (f): contacting the plurality of the retained nucleic acid duplexes of step (e) with a plurality of second sequencing polymerases, wherein the contacting is conducted under a condition suitable for binding the plurality of second sequencing polymerases to the plurality of the retained nucleic acid duplexes, thereby forming a plurality of second complexed polymerases each comprising a second sequencing polymerase bound to a nucleic acid duplex. In some embodiments, the second sequencing polymerase comprises a recombinant mutant sequencing polymerase.

In some embodiments, the plurality of first sequencing polymerases of step (a) have an amino acid sequence that is 100% identical to the amino acid sequence as the plurality of the second sequencing polymerases of step (f). In some embodiments, the plurality of first sequencing polymerases of step (a) have an amino acid sequence that differs from the amino acid sequence of the plurality of the second sequencing polymerases of step (f).

In some embodiments, the methods for sequencing further comprise step (g):
contacting the plurality of second complexed polymerases with a plurality of nucleotides, wherein the contacting is conducted under a condition suitable for binding complementary nucleotides from the plurality of nucleotides to at least two of the second complexed polymerases thereby forming a plurality of nucleotide-complexed polymerases. In some embodiments, the contacting of step (g) is conducted under a condition that is suitable for promoting polymerase-catalyzed incorporation of the bound complementary nucleotides into the primers of the nucleotide-complexed polymerases, thereby extending the sequencing primer by one nucleotide. In some embodiments, the incorporating the nucleotide into the 3′ end of the sequencing primer in step (g) comprises a primer extension reaction. In some embodiments, the contacting of step (g) is conducted in the presence of at least one catalytic cation comprising magnesium and/or manganese. In some embodiments, the plurality of nucleotides comprise native nucleotides (e.g., non-analog nucleotides) or nucleotide analogs. In some embodiments, the plurality of nucleotides comprise a 2′ and/or 3′ chain terminating moiety which is removable. In some embodiments, the plurality of nucleotides comprise a 2′ and/or 3′ chain terminating moiety that is not removable. In some embodiments, at least one of the nucleotides in the plurality is not labeled with a detectable reporter moiety. In some embodiments, the plurality of nucleotides are non-labeled with detectable reporter moieties. In some embodiments, the plurality of nucleotides comprises a plurality of nucleotides labeled with detectable reporter moiety. In some embodiments, detectable reporter moiety comprises a fluorophore. In some embodiments, the fluorophore is attached to the nucleotide base. In some embodiments, the fluorophore is attached to the nucleotide base with a linker which is cleavable/removable from the base or is not removable from the base. In some embodiments, a particular detectable reporter moiety (e.g., fluorophore) that is attached to the nucleotide can correspond to the nucleo-base (e.g., dATP, dGTP, dCTP, dTTP or dUTP) to permit detection and identification of the nucleo-base.

In some embodiments, when the plurality of nucleotides in step (g) are detectably labeled, the methods for sequencing further comprise step (h): detecting the labeled nucleotides which are incorporated into the primers of the nucleotide-complexed polymerases. In some embodiments, the plurality of nucleotides are labeled with a detectable reporter moiety to permit detection. In some embodiments, when the plurality of nucleotides in step (g) are non-labeled, the detecting of step (h) is omitted.

In some embodiments, when the plurality of nucleotides in step (g) are detectably labeled, the methods for sequencing further comprise step (i): identifying the bases of the nucleotides which are incorporated into the primers of the nucleotide-complexed polymerases based on detecting the label, as described above. In some embodiments, the identification of the incorporated nucleotides in step (i) can be used to confirm the identity of the complementary nucleotides of the multivalent molecules that are bound to the plurality of first complexed polymerases in step (d). In some embodiments, the identifying of step (i) can be used to determine the sequence of the nucleic acid template molecules. In some embodiments, when the plurality of nucleotides in step (g) are non-labeled, and the identifying of step (i) is omitted.

In some embodiments, the methods for sequencing further comprise step (j): removing the chain terminating moiety from the incorporated nucleotide when step (g) is conducted by contacting the plurality of second complexed polymerases with a plurality of nucleotides that comprise at least one nucleotide having a 2′ and/or 3′ chain terminating moiety.

In some embodiments, the methods for sequencing further comprise step (k): repeating steps (a)-(j) at least once, e.g. at least 5, 10, 15, 20, 25, 30, 40, 50, 70, 100, 120, 150 or 200 times. In some embodiments, the sequence of the nucleic acid template molecules can be determined by detecting and identifying the multivalent molecules that bind the sequencing polymerases but do not incorporate into the 3′ end of the primer at steps (c) and (d). In some embodiments, the sequence of the nucleic acid template molecule can be determined (or confirmed) by detecting and identifying the nucleotide that incorporates into the 3′ end of the primer at steps (h) and (i).

In some embodiments of the sequencing methods, the binding of the plurality of first complexed polymerases with the plurality of multivalent molecules forms at least one avidity complex, and the method comprising the steps of (a) binding a first nucleic acid primer, a first sequencing polymerase, and a first multivalent molecule to a first portion of a concatemer template molecule thereby forming a first binding complex, wherein a first nucleotide unit of the first multivalent molecule binds to the first sequencing polymerase; and (b) binding a second nucleic acid primer, a second sequencing polymerase, and the first multivalent molecule to a second portion of the same concatemer template molecule thereby forming a second binding complex, wherein a second nucleotide unit of the first multivalent molecule binds to the second sequencing polymerase, wherein the first and second binding complexes which include the same multivalent molecule forms an avidity complex. In some embodiments, the first sequencing polymerase comprises any wild type or mutant polymerase described herein. In some embodiments, the second sequencing polymerase comprises any wild type or mutant polymerase described herein. The concatemer template molecule comprises tandem repeat sequences of a sequence of interest and at least one universal sequencing primer binding site. The first and/or second nucleic acid primers can bind to a sequencing primer binding site along the concatemer template molecule. Exemplary multivalent molecules are shown in FIGS. 11-14, and exemplary single units of concatemer template molecules are shown in FIGS. 9-10.

In some embodiments of the sequencing methods, wherein the method includes binding the plurality of first complexed polymerases with the plurality of multivalent molecules to form at least one avidity complex, the method comprising the steps: (a) contacting the plurality of sequencing polymerases and the plurality of nucleic acid primers with different portions of a concatemer template molecule to form at least first and second complexed polymerases on the same concatemer template molecule; (b) contacting a plurality of multivalent molecules to the at least first and second complexed polymerases on the same concatemer template molecule, under conditions suitable to bind a single multivalent molecule from the plurality to the first and second complexed polymerases, wherein at least a first nucleotide unit of the single multivalent molecule is bound to the first complexed polymerase which includes a first primer hybridized to a first portion of the concatemer template molecule thereby forming a first binding complex (e.g., first ternary complex), and wherein at least a second nucleotide unit of the single multivalent molecule is bound to the second complexed polymerase which includes a second primer hybridized to a second portion of the concatemer template molecule thereby forming a second binding complex (e.g., second ternary complex), wherein the contacting is conducted under a condition suitable to inhibit polymerase-catalyzed incorporation of the bound first and second nucleotide units in the first and second binding complexes, and wherein the first and second binding complexes which are bound to the same multivalent molecule forms an avidity complex; and (c) detecting the first and second binding complexes on the same concatemer template molecule, and (d) identifying the first nucleotide unit in the first binding complex thereby determining the sequence of the first portion of the concatemer template molecule, and identifying the second nucleotide unit in the second binding complex thereby determining the sequence of the second portion of the concatemer template molecule. In some embodiments, the plurality of sequencing polymerases comprise any wild type or mutant sequencing polymerase described herein or known in the art, for example in U.S. Pat. No. 11,859,241. In some embodiments, concatemer template molecule comprises tandem repeat sequences of a sequence of interest and at least one universal sequencing primer binding site.

The plurality of nucleic acid primers can bind to a sequencing primer binding site along the concatemer template molecule. Exemplary multivalent molecules are shown in FIGS. 9-12.

### Sequencing-by-Binding

The present disclosure provides methods for sequencing any of the immobilized template molecules described herein using the sequencing systems, wherein the sequencing methods comprise a sequencing-by-binding (SBB) procedure which employs non-labeled chain-terminating nucleotides. In some embodiments, the sequencing-by-binding (SBB) method comprises the steps of (a) sequentially contacting a primed template nucleic acid molecule (e.g., with single or multiple copies of the sequence of interest as described supra) with at least two separate mixtures under ternary complex stabilizing conditions, wherein the at least two separate mixtures each include a polymerase and a nucleotide, whereby the sequentially contacting results in the primed template nucleic acid being contacted, under the ternary complex stabilizing conditions, with nucleotide cognates for first, second and third base type base types in the template; (b) examining the at least two separate mixtures to determine whether a ternary complex formed; and (c) identifying the next correct nucleotide for the primed template nucleic acid molecule, wherein the next correct nucleotide is identified as a cognate of the first, second or third base type if ternary complex is detected in step (b), and wherein the next correct nucleotide is imputed to be a nucleotide cognate of a fourth base type based on the absence of a ternary complex in step (b); (d) adding a next correct nucleotide to the primer of the primed template nucleic acid molecule after step (b), thereby producing an extended primer; and (e) repeating steps (a) through (d) at least once on the primed template nucleic acid that comprises the extended primer. Exemplary sequencing-by-binding methods are described in U.S. Pat. Nos. 10,246,744 and 10,731,141 (where the contents of both patents are hereby incorporated by reference in their entireties).

### Methods for Sequencing Using Phosphate-Chain Labeled Nucleotides

The present disclosure provides methods for sequencing using the sequencing systems, the methods using immobilized sequencing polymerases which bind non-immobilized template molecules, wherein the sequencing reactions are conducted with phosphate-chain labeled nucleotides. In some embodiments, the sequencing methods comprise step (a): providing a support having a plurality of sequencing polymerases immobilized thereon. In some embodiments, the sequencing polymerase comprises a processive DNA polymerase. In some embodiments, the sequencing polymerase comprises a wild type or mutant DNA polymerase, including for example a Phi29 DNA polymerase. In some embodiments, the support comprises a plurality of separate compartments and a sequencing polymerase is immobilized to the bottom of a compartment. In some embodiments, the separate compartments comprise a silica bottom through which light can penetrate. In some embodiments, the separate compartments comprise a silica bottom configured with a nanophotonic confinement structure comprising a hole in a metal cladding film (e.g., aluminum cladding film). In some embodiments, the hole in the metal cladding has a small aperture, for example, approximately 70 nm. In some embodiments, the height of the nanophotonic confinement structure is approximately 100 nm. In some embodiments, the nanophotonic confinement structure comprises a zero mode waveguide (ZMW). In some embodiments, the nanophotonic confinement structure contains a liquid.

In some embodiments, the sequencing method further comprises step (b): contacting the plurality of immobilized sequencing polymerases with a plurality of single stranded circular nucleic acid template molecules and a plurality of oligonucleotide sequencing primers, under a condition suitable for individual immobilized sequencing polymerases to bind a single stranded circular template molecule, and suitable for individual sequencing primers to hybridize to individual single stranded circular template molecules, thereby generating a plurality of polymerase/template/primer complexes. In some embodiments, the individual sequencing primers hybridize to a universal sequencing primer binding site on the single stranded circular template molecule.

In some embodiments, the sequencing method further comprises step (c): contacting the plurality of polymerase/template/primer complexes with a plurality of phosphate chain labeled nucleotides each comprising an aromatic base, a five carbon sugar (e.g., ribose or deoxyribose), and phosphate chain comprising 3-20 phosphate groups, where the terminal phosphate group is linked to a detectable reporter moiety (e.g., a fluorophore). The first, second and third phosphate groups can be referred to as alpha, beta and gamma phosphate groups. In some embodiments, a particular detectable reporter moiety which is attached to the terminal phosphate group corresponds to the nucleotide base (e.g., dATP, dGTP, dCTP, dTTP or dUTP) to permit detection and identification of the nucleo-base. In some embodiments, the plurality of polymerase/template/primer complexes are contacted with the plurality of phosphate chain labeled nucleotides under a condition suitable for polymerase-catalyzed nucleotide incorporation. In some embodiments, the sequencing polymerases are capable of binding a complementary phosphate chain labeled nucleotide and incorporating the complementary nucleotide opposite a nucleotide in a template molecule. In some embodiment, the polymerase-catalyzed nucleotide incorporation reaction cleaves between the alpha and beta phosphate groups thereby releasing a multi-phosphate chain linked to a fluorophore.

In some embodiments, the sequencing method further comprises step (d): detecting the fluorescent signal emitted by the phosphate chain labeled nucleotide that is bound by the sequencing polymerase, and incorporated into the terminal end of the sequencing primer. In some embodiments, step (d) further comprises identifying the phosphate chain labeled nucleotide that is bound by the sequencing polymerase, and incorporated into the terminal end of the sequencing primer.

In some embodiments, the sequencing method further comprises step (d): repeating steps (c)-(d) at least once. In some embodiments, sequencing methods that employ phosphate chain labeled nucleotides can be conducted according to the methods described in U.S. Pat. Nos. 7,170,050; 7,302,146; and/or 7,405,281.

### Sequencing Polymerases

The present disclosure provides methods for sequencing nucleic acid template molecules, where any of the sequencing methods described herein employ at least one type of sequencing polymerase and a plurality of nucleotides, or employ at least one type of sequencing polymerase and a plurality of nucleotides and a plurality of multivalent molecules. In some embodiments, the sequencing polymerase(s) is/are capable of incorporating a complementary nucleotide opposite a nucleotide in a template molecule. In some embodiments, the sequencing polymerase(s) is/are capable of binding a complementary nucleotide unit of a multivalent molecule opposite a nucleotide in a template molecule. In some embodiments, the plurality of sequencing polymerases comprise recombinant mutant polymerases.

Examples of suitable polymerases for use in sequencing with nucleotides and/or multivalent molecules include, but are not limited to: Klenow DNA polymerase; Thermus aquaticus DNA polymerase I (Taq polymerase); KlenTaq polymerase; Candidatus altiarchaeales archaeon; Candidatus Hadarchaeum Yellowstonense; Hadesarchaea archaeon; Euryarchaeota archaeon; Thermoplasmata archaeon; Thermococcus polymerases such as Thermococcus litoralis, bacteriophage T7 DNA polymerase; human alpha, delta and epsilon DNA polymerases; bacteriophage polymerases such as T4, RB69 and phi29 bacteriophage DNA polymerases; Pyrococcus furiosus DNA polymerase (Pfu polymerase); Bacillus subtilis DNA polymerase III; E. coli DNA polymerase III alpha and epsilon; 9 degree N polymerase; reverse transcriptases such as HIV type M or O reverse transcriptases; avian myeloblastosis virus reverse transcriptase; Moloney Murine Leukemia Virus (MMLV) reverse transcriptase; or telomerase. Further non-limiting examples of DNA polymerases include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases as are known in the art such as 9 degrees N, VENT®, DEEP VENT®, THERMINATOR™, Pfu, KOD, Pfx, Tgo and RB69 polymerases.

Additional polymerases are described, for example, in U.S. Pat. No. 11,891,241, the contents of which are incorporated by reference in their entirety herein.

### Nucleotides

The present disclosure provides methods for sequencing nucleic acid templates molecules, where any of the sequencing methods described herein employ at least one nucleotide, or at least one plurality of nucleotides. The nucleotides comprise a base, sugar and at least one phosphate group. In some embodiments, at least one nucleotide in the plurality comprises an aromatic base, a five carbon sugar (e.g., ribose or deoxyribose), and one or more phosphate groups (e.g., 1-10 phosphate groups). The plurality of nucleotides can comprise at least one type of nucleotide selected from a group consisting of dATP, dGTP, dCTP, dTTP and dUTP. The plurality of nucleotides can comprise at a mixture of any combination of two or more types of nucleotides selected from a group consisting of dATP, dGTP, dCTP, dTTP and/or dUTP. In some embodiments, at least one nucleotide in the plurality is not a nucleotide analog. In some embodiments, at least one nucleotide in the plurality comprises a nucleotide analog.

In some embodiments, in any of the methods for sequencing nucleic acid molecules described herein, at least one nucleotide in the plurality of nucleotides comprise a chain of one, two or three phosphorus atoms where the chain is typically attached to the 5′ carbon of the sugar moiety via an ester or phosphoramide linkage. In some embodiments, at least one nucleotide in the plurality is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

In some embodiments, in any of the methods for sequencing nucleic acid molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide comprising the chain terminating moiety in a nascent strand during a primer extension reaction. In some embodiments, the chain terminating moiety is attached to the 3′ sugar position where the sugar comprises a ribose or deoxyribose sugar moiety. In some embodiments, the chain terminating moiety is removable/cleavable from the 3′ sugar position to generate a nucleotide having a 3′ OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, silyl or acetal group. In some embodiments, the chain terminating moiety is cleavable/removable from the nucleotide, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride. In some embodiments, the chain terminating moiety may be cleavable/removable with nitrous acid. In some embodiments, a chain terminating moiety may be cleavable/removable using a solution comprising nitrite, such as, for example, a combination of nitrite with an acid such as acetic acid, sulfuric acid, or nitric acid. In some further embodiments, said solution may comprise an organic acid.

In some embodiments, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP). In some embodiments, the chain terminating moiety comprising one or more of a 3′-O-amino group, a 3′-O-aminomethyl group, a 3′-O-methylamino group, or derivatives thereof may be cleaved with nitrous acid, through a mechanism utilizing nitrous acid, or using a solution comprising nitrous acid. In some embodiments, the chain terminating moiety comprising one or more of a 3′-O-amino group, a 3′-O-aminomethyl group, a 3′-O-methylamino group, or derivatives thereof may be cleaved using a solution comprising nitrite. In some embodiments, nitrite may be combined with or contacted with an acid such as acetic acid, sulfuric acid, or nitric acid. In some further embodiments, nitrite may be combined with or contacted with an organic acid such as, for example, formic acid, acetic acid, propionic acid, butyric acid, isobutyric acid, or the like. In some embodiments, the chain terminating moiety comprises a 3′-acetal moiety which can be cleaved with a palladium deblocking reagent (e.g., Pd(0)).

In some embodiments, the nucleotide comprises a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, 3-O-benzyl, and 3′-O-benzyl, 3-acetal moiety or derivatives thereof.

In some embodiments the plurality of nucleotides comprises at least one nucleotide labeled with detectable reporter moiety. In some embodiments the plurality of nucleotides comprises a plurality of nucleotides labeled with detectable reporter moiety. In some embodiments, detectable reporter moiety comprises a fluorophore. In some embodiments, the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, the cleavable linker on the nucleotide base, e.g. the linker attaching the nucleotide to the detectable reporter moiety, comprises a cleavable moiety comprising an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the cleavable linker is cleavable/removable from the nucleo-base by reacting the cleavable moiety with a chemical agent, pH change, light or heat. In some embodiments, the cleavable moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the cleavable moieties aryl and benzyl are cleavable with H2 Pd/C. In some embodiments, the cleavable moieties amine, amide, keto, isocyanate, phosphate, thio, disulfide are cleavable with phosphine or with a thiol group including beta-mercaptoethanol or dithiothritol (DTT). In some embodiments, the cleavable moiety carbonate is cleavable with potassium carbonate (K₂CO₃) in MeOH, with triethylamine in pyridine, or with Zn in acetic acid (AcOH). In some embodiments, the cleavable moieties urea and silyl are cleavable with tetrabutylammonium fluoride, pyridine-HF, with ammonium fluoride, or with triethylamine trihydrofluoride.

In some embodiments, the cleavable linker on the nucleotide base comprises a cleavable moiety including an azide, azido or azidomethyl group. In some embodiments, the cleavable moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, in any of the methods for sequencing nucleic acid molecules described herein, the chain terminating moiety (e.g., at the sugar 2′ and/or sugar 3′ position) and the cleavable linker on the nucleotide base have the same or different cleavable moieties. In some embodiments, the chain terminating moiety (e.g., at the sugar 2′ and/or sugar 3′ position) and the detectable reporter moiety linked to the base are chemically cleavable/removable with the same chemical agent. In some embodiments, the chain terminating moiety (e.g., at the sugar 2′ and/or sugar 3′ position) and the detectable reporter moiety linked to the base are chemically cleavable/removable with different chemical agents.

### Multivalent Molecules

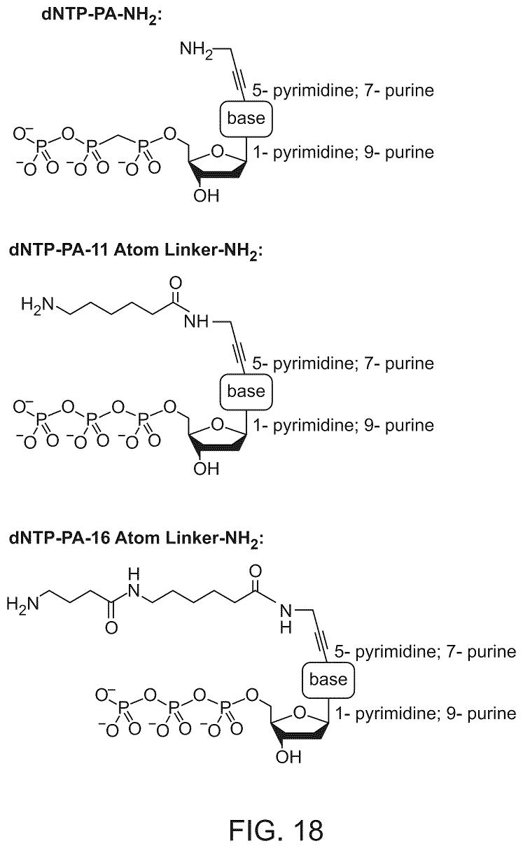

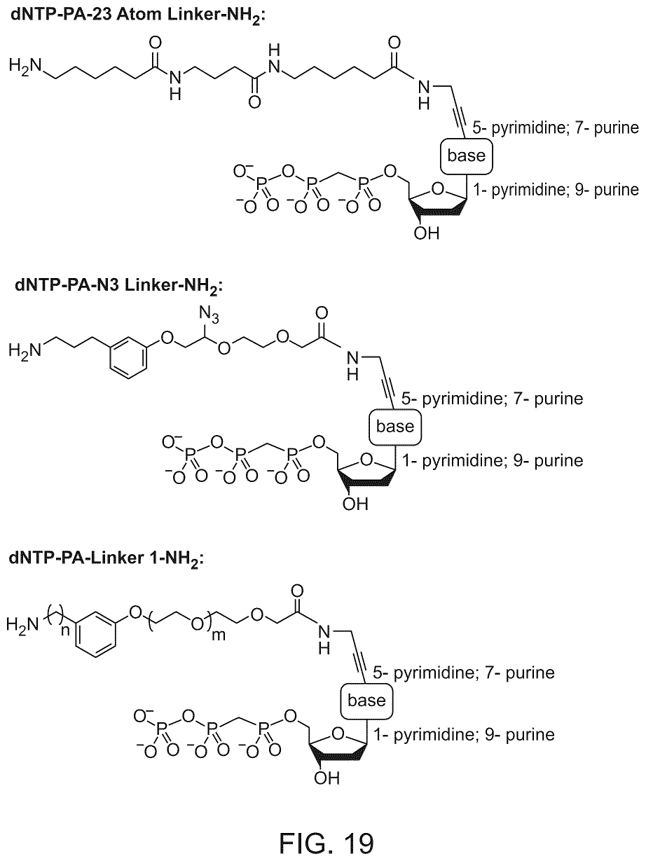

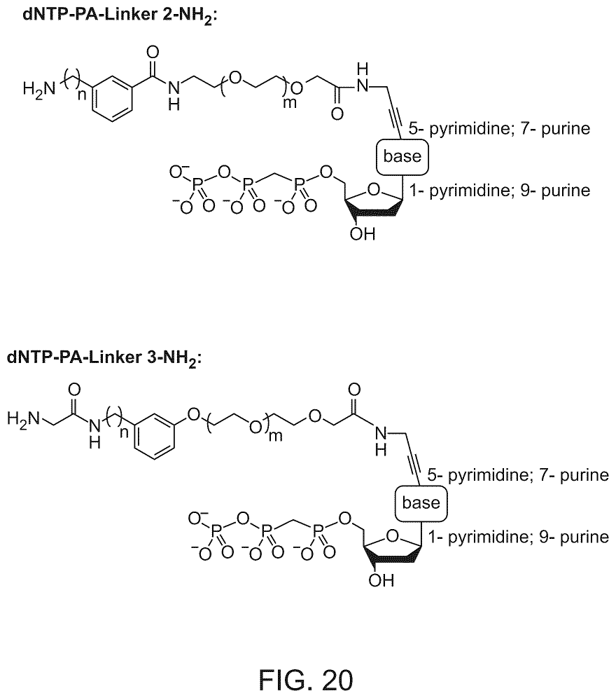

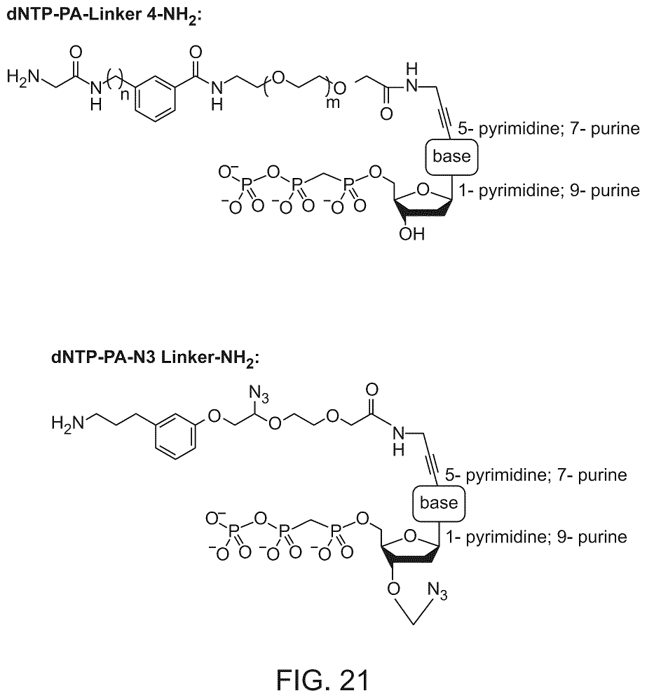

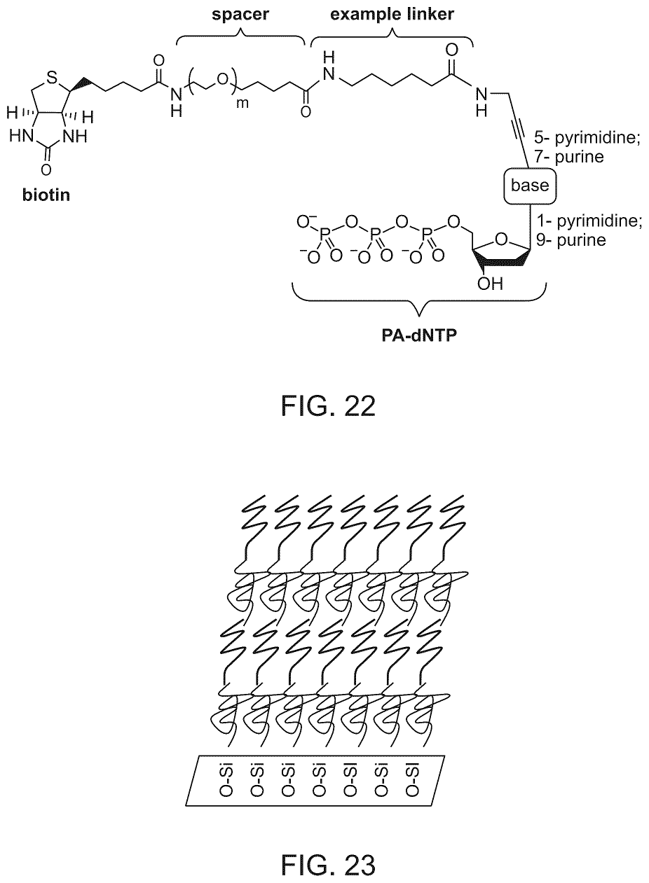

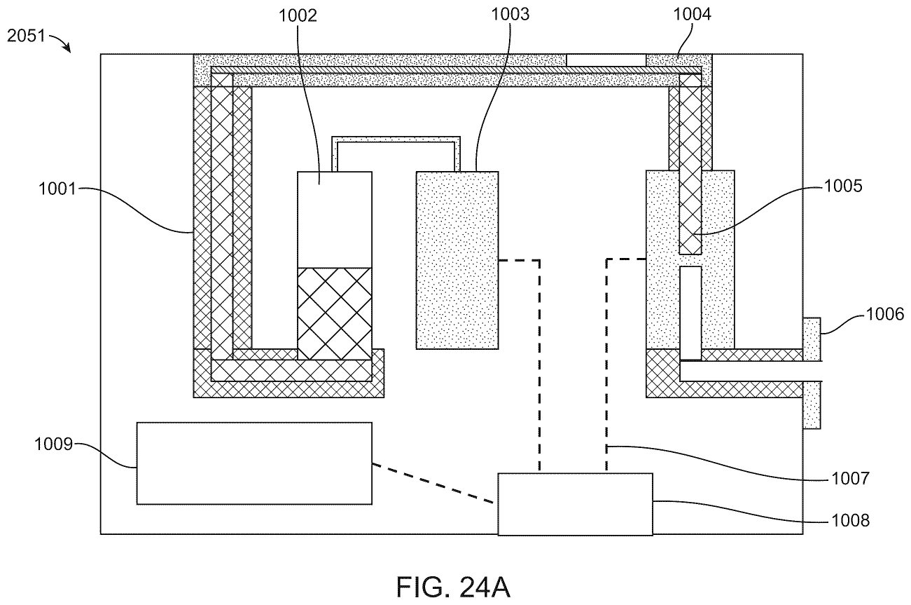

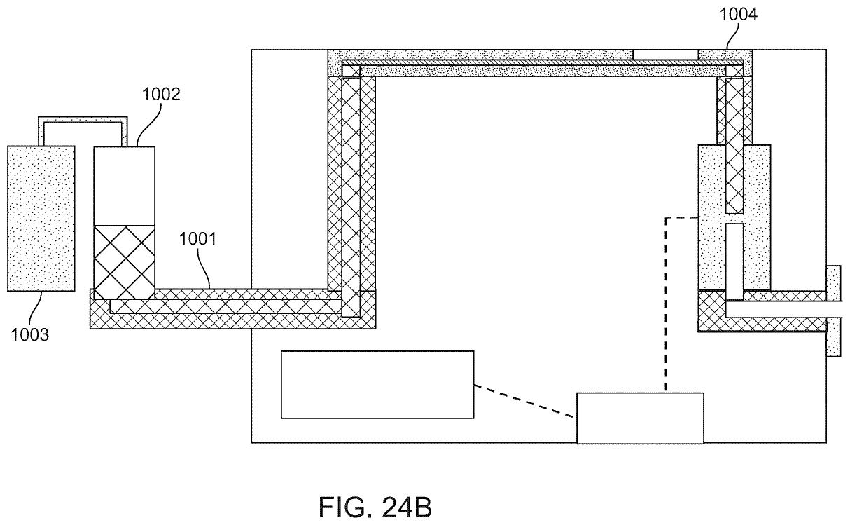

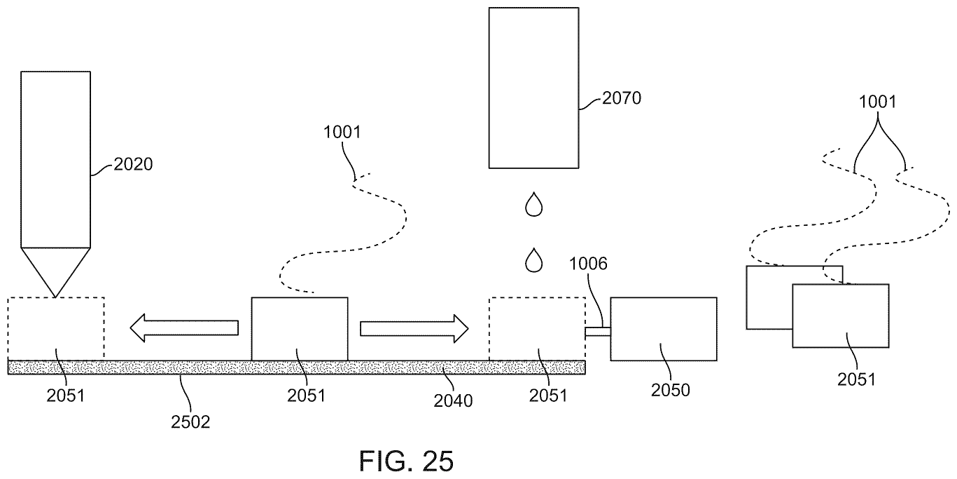

The present disclosure provides methods for sequencing nucleic acid template molecules, where any of the sequencing methods described herein employ at least one multivalent molecule. In some embodiments, the multivalent molecule comprises a plurality of nucleotide arms attached to a core and having any configuration including a starburst, helter skelter, or bottle brush configuration (e.g., FIG. 11). In some embodiments, the multivalent molecule comprises: (1) a core; and (2) a plurality of nucleotide arms which comprise (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, wherein the linker is attached to the nucleotide unit. In some embodiments, the nucleotide unit comprises a base, sugar and at least one phosphate group, and the linker is attached to the nucleotide unit through the base. In some embodiments, the linker comprises an aliphatic chain or an oligo ethylene glycol chain where both linker chains having 2-6 subunits. In some embodiments, the linker also includes an aromatic moiety. An exemplary nucleotide arm is shown in FIG. 15. Exemplary multivalent molecules are shown in FIGS. 11-14. An exemplary spacer is shown in FIG. 16 (top) and exemplary linkers are shown in FIG. 16 (bottom) and FIG. 17. Exemplary nucleotides attached to a linker are shown in FIG. 18-21. An exemplary biotinylated nucleotide arm is shown in FIG. 22.

In some embodiments, a multivalent molecule comprises a core attached to multiple nucleotide arms, and wherein the multiple nucleotide arms have the same type of nucleotide unit which is selected from a group consisting of dATP, dGTP, dCTP, dTTP and dUTP.

In some embodiments, a multivalent molecule comprises a core attached to multiple nucleotide arms, where each arm includes a nucleotide unit. The nucleotide unit comprises an aromatic base, a five carbon sugar (e.g., ribose or deoxyribose), and one or more phosphate groups (e.g., 1-10 phosphate groups). The plurality of multivalent molecules can comprise one type of multivalent molecule having one type of nucleotide unit selected from a group consisting of dATP, dGTP, dCTP, dTTP and dUTP. The plurality of multivalent molecules can comprise at a mixture of any combination of two or more types of multivalent molecules, where individual multivalent molecules in the mixture comprise nucleotide units selected from a group consisting of dATP, dGTP, dCTP, dTTP and/or dUTP.

In some embodiments, the nucleotide unit comprises a chain of one, two or three phosphorus atoms where the chain is typically attached to the 5′ carbon of the sugar moiety via an ester or phosphoramide linkage. In some embodiments, at least one nucleotide unit is a nucleotide analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

In some embodiments, the multivalent molecule comprises a core attached to multiple nucleotide arms, and wherein individual nucleotide arms comprise a nucleotide unit which is a nucleotide analog having a chain terminating moiety (e.g., blocking moiety) at the sugar 2′ position, at the sugar 3′ position, or at the sugar 2′ and 3′ position. In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to the 3′ sugar position where the sugar comprises a ribose or deoxyribose sugar moiety. In some embodiments, the chain terminating moiety is removable/cleavable from the 3′ sugar position to generate a nucleotide having a 3′ OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable/removable from the nucleotide unit, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, the nucleotide unit comprising a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′ butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, the multivalent molecule comprises a core attached to multiple nucleotide arms, wherein the nucleotide arms comprise a spacer, linker and nucleotide unit, and wherein the core, linker and/or nucleotide unit is labeled with detectable reporter moiety. In some embodiments, the detectable reporter moiety comprises a fluorophore. In some embodiments, all fluorophores are on an individual multivalent molecule are the same, i.e., have the same excitation and emission spectra. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, at least one nucleotide arm of a multivalent molecule has a nucleotide unit that is attached to a detectable reporter moiety. In some embodiments, the detectable reporter moiety is attached to the nucleotide base. In some embodiments, the detectable reporter moiety comprises a fluorophore. In some embodiments, a particular detectable reporter moiety (e.g., fluorophore) that is attached to the multivalent molecule can correspond to the identity of the nucleotide base (e.g., dATP, dGTP, dCTP, dTTP or dUTP) of the nucleotide unit(s) to permit detection and identification of the nucleotide base.

In some embodiments, the core of a multivalent molecule comprises an avidin-like or streptavidin-like moiety and the core attachment moiety comprises biotin. In some embodiments, the core comprises an streptavidin-type or avidin-type moiety which includes an avidin protein, as well as any derivatives, analogs and other non-native forms of avidin that can bind to at least one biotin moiety. Other forms of avidin moieties include native and recombinant avidin and streptavidin as well as derivatized molecules, e.g. non-glycosylated avidin and truncated streptavidins. For example, avidin moiety includes de-glycosylated forms of avidin, bacterial streptavidin produced by Streptomyces (e.g., Streptomyces avidinii), as well as derivatized forms, for example, N-acyl avidins, e.g., N-acetyl, N-phthalyl and N-succinyl avidin, and the commercially-available products EXTRAVIDIN®, CAPTAVIDIN™, NEUTRAVIDIN and NEUTRALITE AVIDIN.

In some embodiments of the sequencing methods described herein, the methods can include forming a binding complex, where the binding complex comprises (i) a polymerase, a nucleic acid template molecule duplexed with a primer, and a nucleotide, or the binding complex comprises (ii) a polymerase, a nucleic acid template molecule duplexed with a primer, and a nucleotide unit of a multivalent molecule. In some embodiments, the binding complex has a persistence time of greater than about 0.1, 0.2, 0.3, 0.4, 0.5, 0.6, 0.7, 0.8, 0.9 or 1 second. In some embodiments, the binding complex has a persistence time of greater than about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds, and/or wherein the method is or may be carried out at a temperature of at or above 15° C., at or above 20° C., at or above 25° C., at or above 35° C., at or above 37 ° C., at or above 42° C. at or above 55° C. at or above 60° C., or at or above 72° C., or at or above 80° C., or within a range defined by any of the foregoing. The binding complex (e.g., ternary complex) can remain stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water. In some embodiments, the present disclosure provides said method wherein the binding complex is deposited on, attached to, or hybridized to, a surface showing a contrast to noise ratio in the detecting step of greater than 20. In some embodiments, the present disclosure provides said method wherein the contacting is performed under a condition that stabilizes the binding complex when the nucleotide or nucleotide unit is complementary to a next base of the template nucleic acid, and destabilizes the binding complex when the nucleotide or nucleotide unit is not complementary to the next base of the template nucleic acid.

### Compaction Oligonucleotides

The disclosure provides methods of preparing nucleic acid template molecules for sequencing, for example the concatemer template molecules described herein, which include compaction oligonucleotides. Suitable compaction oligonucleotides are known in the art and are described, for example, in WO2024040058A1, the contents of which are incorporated by reference herein in their entirety.

An exemplary compaction oligonucleotide comprises a single-stranded linear oligonucleotide having a 5′ region that can hybridize to a first portion of a concatemer template molecule and the compaction oligonucleotide having a 3′ region that can hybridize to a second portion of the concatemer template molecule (e.g., the same concatemer template molecule). In some embodiments, hybridization of the compaction oligonucleotides to individual concatemer template molecules causes the concatemer molecule to collapse or fold into a DNA nanoball which is more compact in shape and size compared to a non-collapsed DNA molecule. A spot image of a DNA nanoball can be represented as a Gaussian spot and the size can be measured as a full width half maximum (FWHM). A smaller spot size as indicated by a smaller FWHM typically correlates with an improved image of the spot. In some embodiments, the FWHM of a DNA nanoball spot can be about 10 um or smaller. The DNA nanoball can be a compact nucleic acid structure having a full width half maximum (FWHM) that is smaller compared to a concatemer that is not collapsed/folded into a DNA nanoball.

In some embodiments, compaction oligonucleotides comprise a single stranded oligonucleotides comprising DNA, RNA, or a combination of DNA and RNA. The compaction oligonucleotides can be any length, including 20-150 nucleotides, or 30-100 nucleotides, or 40-80 nucleotides in length.

In some embodiments, the compaction oligonucleotides comprises a 5′ region and a 3′ region, and optionally an intervening region between the 5′ and 3′ regions. The intervening region can be any length, for example about 2-20 nucleotides in length. The intervening region comprises a homopolymer having consecutive identical bases (e.g., AAA, GGG, CCC, TTT or UUU). The intervening region comprises a non-homopolymer sequence.

The 5′ region of the compaction oligonucleotides can be wholly complementary or partially complementary along its length to a first portion of a concatemer template molecule.

The 3′ region of the compaction oligonucleotides can be wholly complementary or partially complementary along its length to a second portion of a concatemer template molecule. The 5′ region of the compaction oligonucleotides can hybridize to a first universal sequence portion of a concatemer template molecule. The 3′ region of the compaction oligonucleotides can hybridize to a second universal sequence portion of a concatemer molecule. The 5′ and 3′ regions of the compaction oligonucleotide can hybridize to the concatemer to pull together distal portions of the concatemer causing compaction of the concatemer to form a DNA nanoball.

The 5′ region of the compaction oligonucleotide can have the same sequence as the 3′ region. The 5′ region of the compaction oligonucleotide can have a sequence that is different from the 3′ region. The 3′ region of the compaction oligonucleotide can have a sequence that is a reverse sequence of the 5′ region.

### Supports and Low Non-Specific Coatings

In some embodiments, the flow cell devices herein in can include a support 210, e.g., a solid support as disclosed herein. The present disclosure provides sequencing compositions and methods which employ a support comprising a plurality of oligonucleotide surface primers immobilized thereon. In some embodiments, the support is passivated with a low non-specific binding coating. The surface coatings described herein exhibit very low non-specific binding to reagents typically used for nucleic acid capture, amplification and sequencing workflows, such as dyes, nucleotides, enzymes, and nucleic acid primers. The surface coatings exhibit low background fluorescence signals or high contrast-to-noise (CNR) ratios compared to conventional surface coatings.

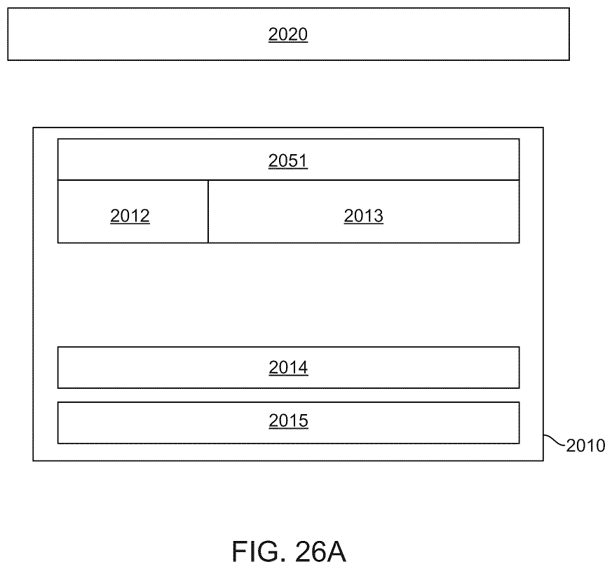

The low non-specific binding coating comprises one layer or multiple layers (FIG. 23). In some embodiments, the plurality of surface primers are immobilized to the low non-specific binding coating. In some embodiments, at least one surface primer is embedded within the low non-specific binding coating. The low non-specific binding coating enables improved nucleic acid hybridization and amplification performance. In general, the supports comprise a substrate (or support structure), one or more layers of a covalently or non-covalently attached low-binding, chemical modification layers, e.g., silane layers, polymer films, and one or more covalently or non-covalently attached surface primers that can be used for tethering single-stranded nucleic acid library molecules to the support. In some embodiments, the formulation of the coating, e.g., the chemical composition of one or more layers, the coupling chemistry used to cross-link the one or more layers to the support and/or to each other, and the total number of layers, may be varied such that non-specific binding of proteins, nucleic acid molecules, and other hybridization and amplification reaction components to the coating is minimized or reduced relative to a comparable monolayer. The formulation of the coating described herein may be varied such that non-specific hybridization on the coating is minimized or reduced relative to a comparable monolayer. The formulation of the coating may be varied such that non-specific amplification on the coating is minimized or reduced relative to a comparable monolayer. The formulation of the coating may be varied such that specific amplification rates and/or yields on the coating are maximized. Amplification levels suitable for detection are achieved in no more than 2, 3, 4, 5, 6, 7, 8, 9, 10, 15, 20, 25, 30, or more than 30 amplification cycles in some cases disclosed herein.

The support structure that comprises the one or more chemically-modified layers, e.g., layers of a low non-specific binding polymer, may be independent or integrated into another structure or assembly. For example, in some embodiments, the support structure may comprise one or more surfaces within an integrated or assembled flow cell device as described herein. The support structure may comprise one or more surfaces within a microplate format, e.g., the bottom surface of the wells in a microplate. In some embodiments, the support structure comprises the interior surface (such as the lumen surface) of a capillary. In some embodiments, the support structure comprises the interior surface (such as the lumen surface) of a capillary etched into a planar chip.

The attachment chemistry used to graft a first chemically-modified layer to the surface of the support will generally be dependent on both the material from which the surface is fabricated and the chemical nature of the layer. In some embodiments, the first layer may be covalently attached to the surface. In some embodiments, the first layer may be non-covalently attached, e.g., adsorbed to the support through non-covalent interactions such as electrostatic interactions, hydrogen bonding, or van der Waals interactions between the support and the molecular components of the first layer. In either case, the support may be treated prior to attachment or deposition of the first layer. Any of a variety of surface preparation techniques known to those of skill in the art may be used to clean or treat the surface. For example, glass or silicon surfaces may be acid-washed using a Piranha solution (a mixture of sulfuric acid (H₂SO4) and hydrogen peroxide (H₂O₂)), base treatment in KOH and NaOH, and/or cleaned using an oxygen plasma treatment method.

Silane chemistries constitute non-limiting approaches for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding coatings include, but are not limited to, (3-Aminopropyl) trimethoxysilane (APTMS), (3-Aminopropyl) triethoxysilane (APTES), any of a variety of PEG-silanes (e.g., comprising molecular weights of 1K, 2K, 5K, 10K, 20K, etc.), amino-PEG silane (i.e., comprising a free amino functional group), maleimide-PEG silane, biotin-PEG silane, and the like.

Any of a variety of molecules known to those of skill in the art including, but not limited to, amino acids, peptides, nucleotides, oligonucleotides, other monomers or polymers, or combinations thereof may be used in creating the one or more chemically-modified layers on the support, where the choice of components used may be varied to alter one or more properties of the layers, e.g., the surface density of functional groups and/or tethered oligonucleotide primers, the hydrophilicity/hydrophobicity of the layers, or the three three-dimensional nature (i.e., “thickness”) of the layer. Examples of polymers that may be used to create one or more layers of low non-specific binding material in any of the disclosed coatings include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the surface and/or to cross-link the layers to each other include, but are not limited to, biotin-streptavidin interactions (or variations thereof), his tag-Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

The low non-specific binding surface coating may be applied uniformly across the support. Alternatively, the surface coating may be patterned, such that the chemical modification layers are confined to one or more discrete regions of the support. For example, the coating may be patterned using photolithographic techniques to create an ordered array or random pattern of chemically-modified regions on the support. Alternately or in combination, the coating may be patterned using, e.g., contact printing and/or ink-jet printing techniques. In some embodiments, an ordered array or random pattern of chemically-modified regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions.

In some embodiments, the low nonspecific binding coatings comprise hydrophilic polymers that are non-specifically adsorbed or covalently grafted to the support. Typically, passivation is performed utilizing poly(ethylene glycol) (PEG, also known as polyethylene oxide (PEO) or polyoxyethylene) or other hydrophilic polymers with different molecular weights and end groups that are linked to a support using, for example, silane chemistry. The end groups distal from the surface can include, but are not limited to, biotin, methoxy ether, carboxylate, amine, NHS ester, maleimide, and bis-silane. In some embodiments, two or more layers of a hydrophilic polymer, e.g., a linear polymer, branched polymer, or multi-branched polymer, may be deposited on the surface. In some embodiments, two or more layers may be covalently coupled to each other or internally cross-linked to improve the stability of the resulting coating. In some embodiments, surface primers, such as the surface capture and surface pinning primers described herein, with different nucleotide sequences and/or base modifications (or other biomolecules, e.g., enzymes or antibodies) may be tethered to the resulting layer at various surface densities. In some embodiments, for example, both surface functional group density and surface primer concentration may be varied to attain a desired surface primer density range. Additionally, surface primer density can be controlled by diluting the surface primers with other molecules that carry the same functional group. For example, amine-labeled surface primers can be diluted with amine-labeled polyethylene glycol in a reaction with an NHS-ester coated surface to reduce the final primer density. Surface primers with different lengths of linker between the hybridization region and the surface attachment functional group can also be applied to control surface density. Example of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of known concentration.

In some embodiments, the low nonspecific binding coatings comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl)acrylamide-co-acrylamide (PAZAM).

In order to scale primer surface density and add additional dimensionality to hydrophilic or amphoteric coatings, supports comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the support significantly. Traditional PEG coating approaches use monolayer primer deposition, which have been generally reported for single molecule applications, but do not yield high copy numbers for nucleic acid amplification applications. As described herein “layering” can be accomplished using traditional crosslinking approaches with any compatible polymer or monomer subunits such that a surface comprising two or more highly crosslinked layers can be built sequentially. Examples of suitable polymers include, but are not limited to, streptavidin, poly acrylamide, polyester, dextran, poly-lysine, and copolymers of poly-lysine and PEG. In some embodiments, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some embodiments, high primer density materials may be constructed in solution and subsequently layered onto the surface in multiple steps.

Examples of materials from which the support structure may be fabricated include, but are not limited to, glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic support structures are contemplated.

The support structure may be rendered in any of a variety of geometries and dimensions known to those of skill in the art, and may comprise any of a variety of materials known to those of skill in the art. For example, the support structure may be locally planar (e.g., comprising a microscope slide or the surface of a microscope slide). Globally, the support structure may be cylindrical (e.g., comprising a capillary or the interior surface of a capillary), spherical (e.g., comprising the outer surface of a non-porous bead), or irregular (e.g., comprising the outer surface of an irregularly-shaped, non-porous bead or particle). In some embodiments, the surface of the support structure used for nucleic acid hybridization and amplification may be a solid, non-porous surface. In some embodiments, the surface of the support structure used for nucleic acid hybridization and amplification may be porous, such that the coatings described herein penetrate the porous surface, and nucleic acid hybridization and amplification reactions performed thereon may occur within the pores. In some embodiments, the support geometry comprises one or more number of channels, inlets and/or outlets as described herein.

The support structure that comprises the one or more chemically-modified layers, e.g., layers of a low non-specific binding polymer, may be independent or integrated into another structure or assembly. For example, the support structure may comprise one or more surfaces within an integrated or assembled microfluidic flow cell. The support structure may comprise one or more surfaces within a microplate format, e.g., the bottom surface of the wells in a microplate. In some embodiments, the support structure comprises the interior surface (such as the lumen surface) of a capillary. In some embodiments the support structure comprises the interior surface (such as the lumen surface) of a capillary etched into a planar chip. In some embodiments, the support comprises one or more inner surfaces of a flow cell device as described herein.

As noted, the low non-specific binding supports of the present disclosure exhibit reduced non-specific binding of proteins, nucleic acids, and other components of the hybridization and/or amplification formulation used for solid-phase nucleic acid amplification. The degree of non-specific binding exhibited by a given support surface may be assessed either qualitatively or quantitatively. For example, exposure of the surface to fluorescent dyes (e.g., cyanins such as Cy3, or Cy5, etc., fluoresceins, coumarins, rhodamines, etc. or other dyes disclosed herein), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a qualitative tool for comparison of non-specific binding on supports comprising different surface formulations. In some embodiments, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a quantitative tool for comparison of non-specific binding on supports comprising different surface formulations-provided that care has been taken to ensure that the fluorescence imaging is performed under conditions where fluorescence signal is linearly related (or related in a predictable manner) to the number of fluorophores on the support surface (e.g., under conditions where signal saturation and/or self-quenching of the fluorophore is not an issue) and suitable calibration standards are used. In some embodiments, other techniques known to those of skill in the art, for example, radioisotope labeling and counting methods may be used for quantitative assessment of the degree to which non-specific binding is exhibited by the different support surface formulations of the present disclosure.

Some surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. Some surfaces disclosed herein exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed be detection of the amount of label remaining on the surface and comparison of the signal resulting therefrom to an appropriate calibration standard. In some embodiments, the label may comprise a fluorescent label. In some embodiments, the label may comprise a radioisotope. In some embodiments, the label may comprise any other detectable label known to one of skill in the art. In some embodiments, the degree of non-specific binding exhibited by a given support surface formulation may thus be assessed in terms of the number of non-specifically bound protein molecules (or nucleic acid molecules or other molecules) per unit area. In some embodiments, the low-binding supports of the present disclosure may exhibit non-specific protein binding (or non-specific binding of other specified molecules, (e.g., cyanins such as Cy3, or Cy5, etc., fluoresceins, coumarins, rhodamines, etc. or other dyes disclosed herein)) of less than 0.001 molecule per μm², less than 0.01 molecule per μm², less than 0.1 molecule per μm², less than 0.25 molecule per μm², less than 0.5 molecule per μm², less than 1 molecule per μm², less than 10 molecules per μm², less than 100 molecules per μm², or less than 1,000 molecules per μm². Those of skill in the art will realize that a given support surface of the present disclosure may exhibit non-specific binding falling anywhere within this range, for example, of less than 86 molecules per um2. For example, some modified surfaces disclosed herein exhibit nonspecific protein binding of less than 0.5 molecule/μm² following contact with a 1 μM solution of Cy3 labeled streptavidin (GE Amersham) in phosphate buffered saline (PBS) buffer for 15 minutes, followed by 3 rinses with deionized water. Some modified surfaces disclosed herein exhibit nonspecific binding of Cy3 dye molecules of less than 0.25 molecules per μm². In independent nonspecific binding assays, 1 μM labeled Cy3 SA (ThermoFisher), 1 μM Cy5 SA dye (ThermoFisher), 10 μM Aminoallyl-dUTP-ATTO-647N (Jena Biosciences), 10 μM Aminoallyl-dUTP-ATTO-Rhol 1 (Jena Biosciences), 10 μM Aminoallyl-dUTP-ATTO-Rhol 1 (Jena Biosciences), 10 μM 7-Propargylamino-7-deaza-dGTP-Cy5 (Jena Biosciences, and 10 μM 7-Propargylamino-7-deaza-dGTP-Cy3 (Jena Biosciences) were incubated on the low binding coated supports at 37° C. for 15 minutes in a 384 well plate format. Each well was rinsed 2-3×0 with 50 ul deionized RNase/DNase Free water and 2-3 × with 25 mM ACES buffer pH 7.4. The 384 well plates were imaged on a GE Typhoon instrument using the Cy3, AF555, or Cy5 filter sets (according to dye test performed) as specified by the manufacturer at a PMT gain setting of 800 and resolution of 50-100 μm. For higher resolution imaging, images were collected on an Olympus IX83 microscope (e.g., inverted fluorescence microscope) (Olympus Corp., Center Valley, Pa.) with a total internal reflectance fluorescence (TIRF) objective (100× , 1.5 NA, Olympus), a CCD camera (e.g., an Olympus EM-CCD monochrome camera, Olympus XM-10 monochrome camera, or an Olympus DP80 color and monochrome camera), an illumination source (e.g., an Olympus 100 W Hg lamp, an Olympus 75 W Xe lamp, or an Olympus U-HGLGPS fluorescence light source), and excitation wavelengths of 532 nm or 635 nm. Dichroic mirrors were purchased from Semrock (IDEX Health & Science, LLC, Rochester, N.Y.), e.g., 405, 488, 532, or 633 nm dichroic reflectors/beamsplitters, and band pass filters were chosen as 532 LP or 645 LP concordant with the appropriate excitation wavelength. Some modified surfaces disclosed herein exhibit nonspecific binding of dye molecules of less than 0.25 molecules per μm². In some embodiments, the coated support was immersed in a buffer (e.g., 25 mM ACES, pH 7.4) while the image was acquired.

In some embodiments, the surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some embodiments, the surfaces disclosed herein exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some embodiments, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surfaced disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

In some embodiments, the hydrophilic surfaces disclosed herein facilitate reduced wash times for bioassays, often due to reduced nonspecific binding of biomolecules to the low-binding surfaces. In some embodiments, adequate wash steps may be performed in less than 60, 50, 40, 30, 20, 15, 10, or less than 10 seconds. For example, adequate wash steps may be performed in less than 30 seconds.

Some low-binding surfaces of the present disclosure exhibit significant improvement in stability or durability to prolonged exposure to solvents and elevated temperatures, or to repeated cycles of solvent exposure or changes in temperature. For example, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some embodiments, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents and/or elevated temperatures (or any combination of these percentages as measured over these time periods). In some embodiments, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes and/or changes in temperature (or any combination of these percentages as measured over this range of cycles).

In some embodiments, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100 fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100 fold greater than a signal of an adjacent amplified nucleic acid population region of the surface.

In some embodiments, fluorescence images of the disclosed low background surfaces when used in nucleic acid hybridization or amplification applications to create polonies of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

One or more types of primer may be attached or tethered to the support surface. In some embodiments, the one or more types of adapters or primers may comprise spacer sequences, adapter sequences for hybridization to adapter-ligated target library nucleic acid sequences, forward amplification primers, reverse amplification primers, sequencing primers, surface capture primers, surface pinning primers and/or molecular barcoding sequences, or any combination thereof. In some embodiments, 1 primer or adapter sequence may be tethered to at least one layer of the surface. In some embodiments, at least 2, 3, 4, 5, 6, 7, 8, 9, 10, or more than 10 different primer or adapter sequences may be tethered to at least one layer of the surface. In some embodiments, the support comprises a plurality of primers tethered to the support, and all the primers comprises the same sequence, e.g. the same surface capture sequence. In some embodiments, the support comprises two or pluralities of primers tethered to the support, and each plurality comprises primers comprising the same sequence, which differs from the corresponding sequence in the other pluralities of primers. As a non-limiting example, a first plurality of primers comprises a first surface capture sequence, a second plurality of primers comprises a second surface capture sequence, and the first and second capture sequences are not the same.

In some embodiments, the tethered adapter and/or primer sequences may range in length from about 10 nucleotides to about 100 nucleotides. In some embodiments, the tethered adapter and/or primer sequences may be at least 10, at least 20, at least 30, at least 40, at least 50, at least 60, at least 70, at least 80, at least 90, or at least 100 nucleotides in length. In some embodiments, the tethered adapter and/or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some embodiments the length of the tethered adapter and/or primer sequences may range from about 20 nucleotides to about 80 nucleotides. Those of skill in the art will recognize that the length of the tethered adapter and/or primer sequences may have any value within this range, e.g., about 24 nucleotides.

In some embodiments, the resultant surface density of primers (e.g., capture primers) on the low binding support surfaces of the present disclosure may range from about 100 primer molecules per μm² to about 100,000 primer molecules per μm². In some embodiments, the resultant surface density of primers on the low binding support surfaces of the present disclosure may range from about 1,000 primer molecules per μm² to about 1,000,000 primer molecules per μm². In some embodiments, the surface density of primers may be at least 1,000, at least 10,000, at least 100,000, or at least 1,000,000 molecules per μm². In some embodiments, the surface density of primers may be at most 1,000,000, at most 100,000, at most 10,000, or at most 1,000 molecules per μm². Any of the lower and upper values described in this paragraph may be combined to form a range included within the present disclosure, for example, in some embodiments the surface density of primers may range from about 10,000 molecules per μm² to about 100,000 molecules per μm². Those of skill in the art will recognize that the surface density of primer molecules may have any value within this range, e.g., about 455,000 molecules per μm². In some embodiments, the surface density of target library nucleic acid sequences initially hybridized to adapter or primer sequences on the support surface may be less than or equal to that indicated for the surface density of tethered primers. In some embodiments, the surface density of clonally-amplified target library nucleic acid sequences hybridized to adapter or primer sequences on the support surface may span the same range as that indicated for the surface density of tethered primers.

Local densities as listed above do not preclude variation in density across a surface, such that a surface may comprise a region having an oligo density of, for example, 500,000/μm², while also comprising at least a second region having a substantially different local density.

In some embodiments, the performance of nucleic acid hybridization and/or amplification reactions using the disclosed reaction formulations and low-binding supports may be assessed using fluorescence imaging techniques, where the contrast-to-noise ratio (CNR) of the images provides a key metric in assessing amplification specificity and non-specific binding on the support. CNR is commonly defined as: CNR=(Signal-Background)/Noise. The background term is commonly taken to be the signal measured for the interstitial regions surrounding a particular feature (diffraction limited spot, DLS) in a specified region of interest (ROI). While signal-to-noise ratio (SNR) is often considered to be a benchmark of overall signal quality, it can be shown that improved CNR can provide a significant advantage over SNR as a benchmark for signal quality in applications that require rapid image capture (e.g., sequencing applications for which cycle times must be minimized), as shown in the example below. At high CNR the imaging time required to reach accurate discrimination (and thus accurate base-calling in the case of sequencing applications) can be drastically reduced even with moderate improvements in CNR. Improved CNR in imaging data on the imaging integration time provides a method for more accurately detecting features such as clonally-amplified nucleic acid colonies on the support surface.

In most ensemble-based sequencing approaches, the background term is typically measured as the signal associated with “interstitial” regions. In addition to “interstitial” background (Bᵢₙₜₑᵣ) , “intrastitial” background (Bᵢₙₜᵣₐ) exists within the region occupied by an amplified DNA colony. The combination of these two background signals dictates the achievable CNR, and subsequently directly impacts the optical instrument requirements, architecture costs, reagent costs, run-times, cost/genome, and ultimately the accuracy and data quality for cyclic array-based sequencing applications. The Bᵢₙₜₑᵣ background signal arises from a variety of sources; a few examples include auto-fluorescence from consumable flow cells, non-specific adsorption of detection molecules that yield spurious fluorescence signals that may obscure the signal from the ROI, the presence of non-specific DNA amplification products (e.g., those arising from primer dimers). In typical next generation sequencing (NGS) applications, this background signal in the current field-of-view (FOV) is averaged over time and subtracted. The signal arising from individual DNA colonies (i.e., (Signal)-B(interstial) in the FOV) yields a discernable feature that can be classified. In some embodiments, the intrastitial background (B(intrastitial)) can contribute a confounding fluorescence signal that is not specific to the target of interest, but is present in the same ROI thus making it far more difficult to average and subtract.

Nucleic acid amplification on the low-binding coated supports described herein may decrease the B(interstitial) background signal by reducing non-specific binding, may lead to improvements in specific nucleic acid amplification, and may lead to a decrease in non-specific amplification that can impact the background signal arising from both the interstitial and intrastitial regions. In some embodiments, the disclosed low-binding coated supports, optionally used in combination with the disclosed hybridization and/or amplification reaction formulations, may lead to improvements in CNR by a factor of 2, 5, 10, 100, 250, 500 or 1000-fold over those achieved using conventional supports and hybridization, amplification, and/or sequencing protocols. Although described here in the context of using fluorescence imaging as the read-out or detection mode, the same principles apply to the use of the disclosed low-binding coated supports and nucleic acid hybridization and amplification formulations for other detection modes as well, including both optical and non-optical detection modes.

In some embodiments, the support is solid, semi-solid, or a combination of both. In some embodiments, the support is porous, semi-porous, non-porous, or any combination of porosity. In some embodiments, the support can be substantially planar, concave, convex, or any combination thereof. In some embodiments, the support can be cylindrical, for example comprising a capillary or interior surface of a capillary.

In some embodiments, the surface of the support can be substantially smooth. In some embodiments, the support can be regularly or irregularly textured, including bumps, etched, pores, three-dimensional scaffolds, or any combination thereof.

In some embodiments, the support comprises a bead having any shape, including spherical, hemi-spherical, cylindrical, barrel-shaped, toroidal, disc-shaped, rod-like, conical, triangular, cubical, polygonal, tubular or wire-like.

The support can be fabricated from any material, including but not limited to glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates are contemplated.

In some embodiments, the surface of the support is coated with one or more compounds to produce a passivated layer on the support. In some embodiments, the support comprises a low non-specific binding surface that enable improved nucleic acid hybridization and amplification performance on the support. In general, the support may comprise one or more layers of a covalently or non-covalently attached low-binding, chemical modification layers, e.g., silane layers, polymer films, and one or more covalently or non-covalently attached oligonucleotides that may be used for immobilizing a plurality of nucleic acid template molecules to the support.

In some embodiments, the degree of hydrophilicity (or “wettability” with aqueous solutions) of the surface coatings may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surfaced disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

The present disclosure provides a plurality (e.g., two or more) of nucleic acid template molecules immobilized to a support. In some embodiments, the plurality of nucleic acid templates have the same sequence or have different sequences. In some embodiments, individual nucleic acid template molecules in the plurality of nucleic acid templates are immobilized to different sites on the support. In some embodiments, two or more individual nucleic acid template molecules in the plurality of nucleic acid templates are immobilized to a site on the support. In some embodiments, the support comprises a plurality of sites arranged in an array. The term “array” refers to a support comprising a plurality of sites located at pre-determined locations on the support to form an array of sites. The sites can be discrete and separated by interstitial regions. In some embodiments, the pre-determined sites on the support can be arranged in one dimension in a row or a column, or arranged in two dimensions in rows and columns. In some embodiments, the plurality of pre-determined sites is arranged on the support in an organized fashion. In some embodiments, the plurality of pre-determined sites is arranged in any organized pattern, including rectilinear, hexagonal patterns, grid patterns, patterns having reflective symmetry, patterns having rotational symmetry, or the like. The pitch between different pairs of sites can be that same or can vary. In some embodiments, the support can have nucleic acid template molecules immobilized at a plurality of sites at a surface density of about 10²-10¹⁵ sites per mm², or more, to form a nucleic acid template array. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are located at pre-determined locations on the support. In some embodiments, a plurality of pre-determined sites on the support (e.g., 10²-10¹⁵ sites or more) are immobilized with nucleic acid template molecules to form a nucleic acid template molecule array. In some embodiments, the nucleic acid template molecules that are immobilized at a plurality of pre-determined sites by hybridization to immobilized surface capture primers, or the nucleic acid template molecules are covalently attached to the surface capture primers. In some embodiments, the nucleic acid template molecules that are immobilized at a plurality of pre-determined sites, for example immobilized at 10²-10¹⁵ sites or more. In some embodiments, the nucleic acid template molecules that are immobilized at a plurality of sites on the support comprise linear or circular nucleic acid template molecules or a mixture of both linear and circular molecules. In some embodiments, the immobilized nucleic acid template molecules are clonally-amplified to generate immobilized nucleic acid polonies at the plurality of pre-determined sites. In some embodiments, individual immobilized nucleic acid template molecules comprise one copy of a target sequence of interest, or comprise concatemer template moleculess having two or more tandem copies of a sequence of interest.

In some embodiments, a support comprising a plurality of sites located at random locations on the support is referred to herein as a support having randomly located sites thereon. The location of the randomly located sites on the support are not pre-determined. The plurality of randomly-located sites can be arranged on the support in a disordered and/or unpredictable fashion. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are randomly located on the support. In some embodiments, a plurality of randomly located sites on the support (e.g., 10²-10¹⁵ sites or more) comprise nucleic acid template molecules to form a support immobilized with nucleic acid template molecules. In some embodiments, the nucleic acid template molecules that are immobilized at a plurality of randomly located sites by hybridization to immobilized surface capture primers, or the nucleic acid template molecules are covalently attached to the surface capture primers. In some embodiments, the nucleic acid template molecules are immobilized at a plurality of randomly located sites, for example immobilized at 10²-10¹⁵ sites or more. In some embodiments, the nucleic acid template moleculess that are immobilized at a plurality of sites on the support comprise linear or circular nucleic acid template molecules, or a mixture of both linear and circular molecules. In some embodiments, the immobilized nucleic acid template molecules are clonally-amplified to generate immobilized nucleic acid polonies at the plurality of randomly located sites. In some embodiments, individual immobilized nucleic acid template molecules comprise one copy of a sequence of interest, or comprise concatemer template molecules having two or more tandem copies of a sequence of interest.

In some embodiments, the plurality of nucleic acid template molecules immobilized on the support are in fluid communication with each other to permit flowing a solution of reagents using the sequencing system 110 described herein (e.g., enzymes including polymerases, multivalent molecules, nucleotides, divalent cations and/or buffers and the like) onto the support so that the plurality of nucleic acid template molecules on the support can be reacted with the reagents in a massively parallel manner. In some embodiments, the fluid communication of the plurality of nucleic acid template molecules can be used to conduct nucleotide binding assays and/or conduct nucleotide polymerization reactions (e.g., primer extension or sequencing) on the plurality of nucleic acid template molecules, and to conduct detection and imaging for massively parallel sequencing. As used herein with respect to nucleic acids and proteins, term “immobilized” and related terms refer to nucleic acid molecules or proteins (e.g., polymerases and other enzymes) that are attached to the support at pre-determined or random locations, where the nucleic acid molecules or proteins are attached directly to a support through covalent bond or non-covalent interaction, or the nucleic acid molecules or proteins are attached to a coating on the support.

When used in reference to a low binding surface coating, one or more layers of a multi-layered surface coating may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched ), poly(acrylic acid) (branched PAA), branched polyacrylamide, branched poly(N-isopropylacrylamide) (branched PNIPAM), branched poly(methyl methacrylate) (branched PMA), branched poly(2-hydroxylethyl methacrylate) (branched PHEMA), branched poly(oligo(ethylene glycol) methyl ether methacrylate) (branched POEGMA), branched polyglutamic acid (branched PGA), branched poly-lysine, branched poly-glucoside, and dextran.

In some embodiments, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branched.

Linear, branched, or multi-branched polymers used to create one or more layers of any of the multi-layered surfaces disclosed herein may have a molecular weight of at least 500, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some embodiments, e.g., wherein at least one layer of a multi-layered surface comprises a branched polymer, the number of covalent bonds between a branched polymer molecule of the layer being deposited and molecules of the previous layer may range from about one covalent linkage per molecule and about 32 covalent linkages per molecule. In some embodiments, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 covalent linkages per molecule.

Any reactive functional groups that remain following the coupling of a material layer to the surface may optionally be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface, may range from 1 to about 10. In some embodiments, the number of layers is at least 1, at least 2, at least 3, at least 4, at least 5, at least 6, at least 7, at least 8, at least 9, or at least 10. In some embodiments, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some embodiments the number of layers may range from about 2 to about 4. In some embodiments, all of the layers may comprise the same material. In some embodiments, each layer may comprise a different material. In some embodiments, the plurality of layers may comprise a plurality of materials. In some embodiments at least one layer may comprise a branched polymer. In some embodiment, all of the layers may comprise a branched polymer.

One or more layers of low non-specific binding material may in some cases be deposited on and/or conjugated to the substrate surface using a polar protic solvent, a polar or polar aprotic solvent, a nonpolar solvent, or any combination thereof. In some embodiments the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino)propanesulfonic acid (MOPS), etc.), or any combination thereof. In some embodiments, an organic component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of water or an aqueous buffer solution. In some embodiments, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of an organic solvent. The pH of the solvent mixture used may be less than 6, about 6, 6.5, 7, 7.5, 8, 8.5, 9, or greater than pH 9.

The term “branched polymer” and related terms refers to a polymer having a plurality of functional groups that help conjugate a biologically active molecule such as a nucleotide, and the functional group can be either on the side chain of the polymer or directly attaches to a central core or central backbone of the polymer. The branched polymer can have linear backbone with one or more functional groups coming off the backbone for conjugation. The branched polymer can also be a polymer having one or more sidechains, wherein the side chain has a site suitable for conjugation. Examples of the functional group include but are limited to hydroxyl, ester, amine, carbonate, acetal, aldehyde, aldehyde hydrate, alkenyl, acrylate, methacrylate, acrylamide, active sulfone, hydrazide, thiol, alkanoic acid, acid halide, isocyanate, isothiocyanate, maleimide, vinylsulfone, dithiopyridine, vinylpyridine, iodoacetamide, epoxide, glyoxal, dione, mesylate, tosylate, and tresylate.

It is to be appreciated that the Detailed Description section, and not any other section, is intended to be used to interpret the claims. Other sections may set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries may be defined as long as the specified functions and relationships (or equivalents thereof) are appropriately performed. Also, alternative embodiments may perform functional blocks, steps, operations, methods, etc. using orderings different from those described herein.

References herein to “one embodiment,” “an embodiment,” “an example embodiment,” “some embodiments,” or similar phrases, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein.

Additionally, some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

While preferred embodiments of the present invention have been shown and described herein, it will be obvious to those skilled in the art that such embodiments are provided by way of example only. The breadth and scope of this disclosure should not be limited by any of the above-described exemplary embodiments. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A sequencing system comprising:
an optical system 2020 comprising an objective lens;
a x-y stage 2010 configured to hold a sample to be imaged thereon and to move the sample within an x-y plane relative to the objective lens, wherein the sample is immobilized on one or more flow cell devices;
a nest bank 2050 configured to provide fluidic and thermal communication to the sample when the one or more flow cell devices are coupled to the nest bank; and
a moving mechanism 2040, optionally comprising a movable arm configured to move the one or more flow cell devices between the x-y stage 2010 and the nest bank 2050 during a sequence run.

2. The sequencing system of claim 1, wherein the x-y stage 2010 is actuated automatically by a first actuator with a first spatial precision.

3. The sequencing system of claim 1 or 2, wherein the movable arm is actuated automatically by a second actuator with a second spatial precision.

4. The sequencing system of any one of the preceding claims, wherein the first actuator, the second actuator, or both is controlled by one or more hardware processors of the sequencing system.

5. The sequencing system of any one of the preceding claims, wherein the sequencing system further comprises:
a housing configured for holding one or more of the optical system 2020, the x-y stage 2010, the nest bank 2050, and the moving mechanism 2040 therewithin.

6. The sequencing system of any one of the preceding claims, wherein the movable arm is actuated automatically to move in three dimensions (3D).

7. The sequencing system of any one of the preceding claims, wherein movement in each of the three dimensions are of one or more predetermined spatial precision.

8. The sequencing system of any one of the preceding claims, wherein the sequencing system lacks fluidic communication or thermal communication at or near the x-y stage 2010 to the one or more flow cell devices when the flow cell devices are immobilized on the x-y stage 2010.

9. The sequencing system of any one of the preceding claims, wherein each of the one or more flow cell devices comprises an open landing area configured for receiving fluids openly from the nest bank 2050.

10. The sequencing system of any one of the preceding claims, wherein the flow cell device comprises a plurality of microfluidic channels, and the nest bank 2050 is configured to allow fluidic communication to each of the plurality of microfluidic channel independently and simultaneously.

11. The sequencing system of any one of the preceding claims, wherein the flow cell device comprises a plurality of microfluidic channels, and the nest bank 2050 is configured to allow fluidic communication to each of the plurality of microfluidic channel independently and sequentially.

12. The sequencing system of any one of the preceding claims, wherein the flow cell device comprises a plurality of microfluidic channels, and the nest bank 2050 is configured to allow fluidic communication to each of the plurality of microfluidic channel independently without cross-contamination.

13. The sequencing system of any one of the preceding claims, wherein the x-y stage 2010 is actuated to move within the x-y plane for a predetermined distance.

14. The sequencing system of any one of the preceding claims, wherein the predetermined distance is based on the distance between two adjacent microfluidic channels of the flow cell device.

15. The sequencing system of any one of the preceding claims, wherein the nest bank 2050 is configured to enable fluidic and thermal communication with the one or more flow cell devices.

16. The sequencing system of any one of the preceding claims, wherein the nest bank 2050 is configured to enable fluidic and thermal communication with at least 1, 2, 3, 4, 5, 6, 7, 8, 9 or 10 flow cell devices when each of the flow cell devices is in a locked position with the nest bank 2050.

17. The sequencing system of any one of the preceding claims, wherein the nest bank 2050 is configured to hold each of the flow cell devices in a unlocked position in which the flow cell device is removable from the nest bank 2050 and a locked position in which the flow cell device is spatially registered to the nest bank 2050, fixedly coupled to the nest bank 2050, and sealed fluidic communication and thermal communication between the nest bank and the flow cell device are enabled.

18. The sequencing system of any one of the preceding claims, wherein the flow cell device is coupled to a carrier 2051.

19. The sequencing system of any one of the preceding claims, wherein the movable arm is configured to move the carrier 2051 and the flow cell device together.

20. The sequencing system of any one of the preceding claims, wherein the carrier 2051 is configured to be spatially registered to the nest bank in the locked position.

21. The sequencing system of any one of the preceding claims, wherein the nest bank 2050 comprises one or more fasteners.

22. The sequencing system claim 21, wherein the one or more fasteners use magnetic force.

23. The sequencing system claim 21 or 22, wherein the one or more fasteners comprises a rare earth magnet, an electromagnetic coil, or both.

24. The sequencing system of any one of claims 21-23, wherein the one or more fasteners are controlled by one or more processors to switch between a on-stage and an off-stage.

25. The sequencing system of any one of claims 21-24, wherein the one or more fasteners lack mechanical fasteners.

26. The sequencing system of any one of the preceding claims, wherein the movable arm is configured to move the one or more flow cell devices between the x-y stage 2010 and the nest bank 2050 with a first spatial precision.

27. The sequencing system of any one of the preceding claims, wherein the movable arm comprises a grabber that is configured to grab a carrier 2051 when the carrier 2051 is in a decoupled position in relation to the nest bank 2050 or when the carrier 2051 is in the decoupled position in relation to the x-y stage 2010.

28. The sequencing system of any one of the preceding claims, wherein the movable arm comprises a horizontal arm that is mechanically supported by a vertical arm.

29. The sequencing system of any one of the preceding claims, wherein the movable arm comprises an upper arm, a joint, a forearm, a wrist, and a grabber attached to the forearm.

30. The sequencing system of any one of the preceding claims, wherein the movable arm is configured to move with 6 degrees of freedom.

31. The sequencing system of any one of claims 27-30, wherein the grabber is movably attached to the horizontal or vertical arm.

32. The sequencing system of any one of claims 27-31, wherein the grabber is configured to move in 3D.

33. The sequencing system of any one of the preceding claims, wherein the moving mechanism 2040 comprises a plurality of tracks, each track connecting a carrier 2051 coupled to the nest bank to the x-y stage 2010.

34. The sequencing system of any one of the claims 27-32, wherein the grabber is configured to hold the flow cell device carrier via frictional, electromagnetic, or magnetic force.

35. The sequencing system of any one of the preceding claims, wherein the carrier 2051 comprises one or more sensors.

36. The sequencing system of any one of the preceding claims, wherein the x-y stage 2010 comprises one or more sensors.

37. The sequencing system of any one of the preceding claims, wherein the nest bank 2050 comprises one or more sensors.

38. The sequencing system of any one of claims 35-37, wherein the one or more sensors are configured to provide feedback to a processor that facilitates positioning of the carrier 2051 relative to the x-y stage 2010, the optical system 2020, or the nest bank 2050.

39. The sequencing system of any one of the preceding claims, wherein the moving mechanism 2040 comprises one or more belt conveyors.

40. The sequencing system of any one of claims 33-39, wherein the plurality of tracks comprises one or more actuators configured to actuate one or more of the plurality of tracks to move corresponding carriers 2051 to the x-y stage 2010.

41. The sequencing system of any one of the preceding claims, wherein the x-y stage 2010 is configured to be actuated to move to a 3D position with a second spatial precision.

42. The sequencing system of claim 41, wherein the second spatial precision is greater than the first spatial precision by 2×, 3×, 4×, 5×, 6×, 7×, 8×, 9×, or 10×.

43. The sequencing system of any one of the preceding claims, wherein the x-y stage 2010 comprises:
a fastener configured to removably secure the flow cell device thereto.

44. The sequencing system of claim 43, wherein the fastener comprises one or more clamps.

45. The sequencing system of any one of the preceding claims, wherein each carrier 2051 comprises a coupled position in which the carrier 2051 is removably attached to the x-y stage 2010.

46. The sequencing system of any one of the preceding claims, wherein each carrier 2051 comprises a decoupled position in which the carrier 2051 is removable from the x-y stage 2010.

47. The sequencing system of any one of the preceding claims, wherein the x-y stage 2010 comprises one or more pumps configured to extract fluids from the flow cell device when the corresponding carrier 2051 is coupled to the x-y stage 2010.

48. The sequencing system of any one of the preceding claims, wherein the x-y stage 2010 comprises a heating device, a cooling device, or both.

49. The sequencing system of any one of the preceding claims, wherein the x-y stage 2010 is coupled to a mechanical decoupler that is configured to isolate the x-y stage from vibration or mechanical disturbance external to the x-y stage 2010.

50. The sequencing system of any one of the preceding claims, wherein the nest bank 2050 comprises one or more fasteners, each configured to fasten a corresponding carrier 2051 to the nest bank 2050.

51. The sequencing system of any one claims 21-50, wherein each fastener comprises one or more clamps.

52. The sequencing system of claim 51, wherein the one or more clamps are actuated by magnetic or electromagnetic force or pressure.

53. The sequencing system of any one of the preceding claims, wherein the nest bank 2050 comprises one or more pumps configured to extract fluids from the flow cell device when the corresponding carrier 2051 is coupled to the nest bank 2050.

54. The sequencing system of any one of the preceding claims, wherein each carrier 2051 comprises a decoupled position in which the carrier 2051 is removable from the nest bank 2050.

55. The sequencing system of any one of the preceding claims, wherein each carrier 2051 comprises a coupled position in which the flow cell device carrier is removably attached to the nest bank 2050, and in sealed fluidic communication with the nest bank 2050.

56. The sequencing system of any one of the preceding claims, wherein the nest bank 2050 comprises a 3D movement device that is configured to position the carrier 2051 relative to the nest bank with a third spatial precision.

57. The sequencing system of claim 56, wherein the third spatial position is greater than the first spatial precision by 2×, 3×, 4×, 5×, 6×, 7×, 8×, 9×, or 10×.

58. The sequencing system of any one of the preceding claims, wherein the carrier 2051 comprises:
an opening at a surface of the carrier 2051 configured to receive a flow cell device therein.

59. The sequencing system of any one of the preceding claims, wherein the carrier 2051 comprises:
one or more fluidic pathways in sealed fluidic communication with the flow cell device when the flow cell device is removably attached to the carrier 2051.

60. The sequencing system of any one of the preceding claims, wherein the carrier 2051 comprises:
a pump configured to pull or push fluids between the flow cell device and the carrier 2051.

61. The sequencing system of any one of the preceding claims, wherein the carrier 2051 comprises:
a valve positioned between a fluidic pathway connecting to the flow cell device and a port opening of the carrier 2051, wherein the valve that is in an open position when the flow cell device is in the coupled position to the carrier 2051; and in a closed position when the flow cell device is in the decoupled position.

62. The sequencing system of any one of the preceding claims, wherein the carrier 2051 comprises:
a port opening with a connector that is configured to enable sealed fluidic communication between the carrier 2051 and the corresponding nest module when the connector is in a connected position.

63. The sequencing system of any one of the preceding claims, wherein the carrier 2051 comprises:
electric wiring with an electric connector configured to enable electric communication between the carrier 2051 and a power supply.

64. The sequencing system of any one of the preceding claims, wherein the carrier 2051 comprises: a battery, a sensor, or both, and wherein the battery or sensor is connected with the electric connector via the electric wiring.

65. The sequencing system of any one of the preceding claims, wherein the nest bank 2050 comprises one or more reagent containers.

66. The sequencing system of any one of the preceding claims, wherein the one or more reagent containers are disposable.

67. The sequencing system of claim 65 or 66, wherein the moving mechanism 2040 is configured to dip a flow cell device into at least some of the one or more reagent containers.

68. The sequencing system of any one of the preceding claims, wherein the nest bank 2050 further comprises: a cooler, a heater, or both.

69. The sequencing system of claim 68, wherein the cooler or heater is configured to control temperature of each sample immobilized on the one or more flow cell devices.

70. The sequencing system of claim 68 or 69, wherein the cooler or heater comprises one or more of: a fan configured to blow cool or hot air; a microwave, an infrared light source, and an electromagnetic wave source.

71. The sequencing system of any one of the preceding claims further comprising a beam dump configured to absorb at least some excitation light generated by the optical system.

72. sequencing system of any one of the preceding claims further comprising a beam dump configured to prevent at least some excitation light from reaching an imaging sensor of the optical system.

73. The sequencing system of claim 71 or 72, wherein the beam dump is displaced from the flow cell device by a gap zone.

74. The sequencing system of any one of claims 71-73, wherein the beam dump contacts the flow cell device with a predetermined latching force.

75. The sequencing system of any one of claims 71-74, wherein the beam dump contacts the x-y stage with a predetermined damping force.

76. The sequencing system of claim 75, wherein the predetermined damping force is configured to reduce the predetermined latching force so that a net force on the flow cell device can be customized to be within a predetermined range.

77. A sequencing method comprising:
(a) moving a first flow cell device from a nest bank 2050 to a x-y stage 2010, wherein the first flow cell device comprises a first sample immobilized thereon;
(b) moving the x-y stage 2010 and the first sample thereon relative to an objective lens of an optical system of a sequencing system;
(c) imaging the first sample immobilized on the first flow cell device on the x-y stage using the optical system 2020;
(d) moving the first flow device from the x-y stage 2010 to the nest bank 2050;
(e) simultaneously allowing fluidic and thermal communication between the nest bank 2050 and a second flow cell device during one or more of: (a)-(d);
(f) moving a second flow cell device from the nest bank 2050 to the x-y stage 2010, wherein the second flow cell device comprises a second sample immobilized thereon;
g) moving the x-y stage 2010 and the second sample thereon relative to an objective lens of the optical system 2040 of the sequencing system;
(h) imaging the second sample immobilized on the second flow cell device on the x-y stage 2010 using the optical system 2040;
(i) moving the first flow device from the x-y stage 2010 to the nest bank 2050; and
(j) simultaneously allowing fluidic and thermal communication between the nest bank 2050 and the first flow cell device during one or more of: (f)-(i).

78. A sequencing method comprising:
(a) moving a first flow cell device from a nest bank 2050 to a x-y stage 2010, wherein the first flow cell device comprises a first sample immobilized thereon;
(b) moving the x-y stage 2010 and the first sample thereon relative to an objective lens of an optical system 2020 of a sequencing system;
(c) imaging the first sample immobilized on the first flow cell device on the x-y stage 2010 using the optical system 2020;
(d) moving the first flow device from the x-y stage 2010 to the nest bank 2050;
(e) simultaneously allowing fluidic and thermal communication between the nest bank 2050 and a second flow cell device during one or more of: (a)-(d); and
(f) moving the second flow cell device from the nest bank 2050 to the x-y stage 2010, wherein the second flow cell device comprises a second sample immobilized thereon.

79. The method of claim 77 or 78, wherein the sequencing method further comprising:
repeating operations (a)-(e).

80. The method of claim 78 or 79, wherein the sequencing method further comprising:
repeating operations (f)-(j).

81. The method of any one of claims 78-80, wherein the sequencing method further comprising:
repeating operations (a)-(j) for a number of repetitions.

82. The method of any one of claims 79-81, wherein the number of repetitions is in a range from 1 to 500.

83. The method of any one of claims 77-82, wherein allowing fluidic communication between the nest bank 2050 and the first flow cell device comprises:
reversibly fastening the flow cell device to a carrier 2051 via one or more fasteners to enable sealed fluidic communication between the flow cell device and the carrier 2051; and
reversibly fastening the carrier 2051 to the nest bank 2050 via the one or more fasteners to enable sealed fluidic communication between the nest bank 2050 and the carrier 2051 and to enable physical contact to heat dissipation elements.

84. The method of any one of claims 77-83, wherein (a) moving the first flow cell device from the nest bank 2050 to the x-y stage 2010 is within a first flow cycle of a sequence run and (f) moving the first flow cell device from the nest bank 2050 to the x-y stage 2010 is within a second flow cycle of the sequencing run different from the first flow cycle.

85. The method of any one of claims 77-84, wherein each of the operations of: (a)-(b) and (d)-(g) is completed within less than 0.5 seconds, 1 second, 2 seconds, 3 seconds, 4 seconds, 5 seconds, 6 seconds, 7 seconds, 8 seconds, 9 seconds, or 10 seconds.

86. The method of any one of claims 77-85, wherein each of the operations of: (a)-(b) and (d)-(g) is completed within less than 0.5 seconds, 1 second, 2 seconds, or 3 seconds.

87. The method of any one of claims 77-86, wherein (e) simultaneously allowing fluidic and thermal communication between the nest bank 2050 and the first flow cell device during one or more of: (a)-(d) comprises:
turning the one or more fasteners into an on-stage to enable sealed fluidic communication and physical contact for thermal communication.

88. The method of any one of claims 77-87, wherein (e) simultaneously allowing fluidic and thermal communication between the nest bank and the first flow cell device during one or more of: (a)-(d) comprises:
dipping the flow cell device into at least some of the one or more reagent containers in a predetermined sequence.
